SEQUENCE LISTING (50 sequences)

SEQ ID NO: 1 - protein, 432 aa, Artificial Sequence
  Nivolumab full length heavy chain
QVQLVESGGGWQPGRSLRLDCKASGITFSNSGMHWVRQAPGKGLEWVAVRWYDGSKRYYADSVKGRFTISRDNSKNTLFLQMNSLRAEDTAVYYCATNDDYWGQGTLVTVSSASTKGPSVFPLAPCSRSTSESTAALGCLVDYFPEPVTVSWNSGALTSGVHTFPAVLQSSGLYSLSSVVTVPSSSLGTTYTCNVDHKPSNTKVDRVESYGPPCPPCPAPEFLGGPSVFLFPPKPKDTLMISRTPEVTCWVDVSQEDPEVQFNWYYDGVEVHNATKPREEQFNSTYRVVSVLTVLHQDWLNGKEYKCKVSNKGLPSSIEKTISKAGQPREPQVYTLPPSQEEMTKNQVSLTCLVKGFYPSDIAVEWESNGQPEKNYKTTPPVLDSDGSFFLYSRLTVDKSRWQEGNVFSCSVMHEALHNHYTQKSLSLSLGK

SEQ ID NO: 2 - protein, 208 aa, Artificial Sequence
  Nivolumab full length light chain
EIVLTQSPATLSLSPGERATLSCRASQSVSSYLAWYQQPGQAPRLLIYDASNRATGIPARFSGSGSGTDFTLTISSLEPEDFAVYYCQQSSNWPRTFGQGTKVEIRTVAAPSVFIFPPSDEQLSGTASVVCLLNNFYPREAVQWKVDNALQSGNSQESVTEQDSDSTYSLSSTLTLSKADYEKHKVYACEVTHQGLSSPVTSFNRGEC

SEQ ID NO: 3 - protein, 447 aa, Artificial Sequence
  pembrolizumab heavy chain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 218 aa, Artificial Sequence
  pembrolizumab light chain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 113 aa, Artificial Sequence
  humanized light chain variable domain sequence
DIVMTQSPDSLAVSLGERATINCKSSQSLWDSGNQKNFLTWYQQKPGQPPKLLIYWTSTRESGVPDRFSGSGSGTDFTLTISSLQAEDVAVYYCQNDYFYPLTFGGGTKVEIK

SEQ ID NO: 6 - protein, 117 aa, Artificial Sequence
  humanized heavy chain variable domain sequence
QVQLVQSGAEVKKPGASVKVSCKASGYTFTSYWINWVRQAPGQGLEQMGNIYPGSSLTNYNEKFKNRVTMTRDTSTSTVYMELSSLRSEDTAVYYCARLLTGTFAYWGQGTLVTVSS

SEQ ID NO: 7 - protein, 113 aa, Artificial Sequence
  humanized light chain variable domain sequence
DIVMTQSPDSLAVSLGERATINCKSSQSLWDSGNQKNFLTWYQQKPGQPPKLLIYWTSYRESGVPDRFSGSGSGTDFTLTISSLQAEDVAVYYCQNDYFYPLTFGGGTKVEIK

SEQ ID NO: 8 - protein, 117 aa, Artificial Sequence
  humanized heavy chain variable domain
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLLTGTFAYWGQGTLVTVSS

SEQ ID NO: 9 - protein, 113 aa, Artificial Sequence
  humanized light chain variable domain
DIVMTQSPDSLAVSLGERATINCKSSQSLWDSGNQKNFLTWYQQKPGQPPKLLIYWTSYRESGVPDRFSGSGSGTDFTLTISSLQAEDVAVYYCQNDYFYPHTFGGGTKVEIK

SEQ ID NO: 10 - protein, 117 aa, Artificial Sequence; identical to SEQ ID NO: 8
  humanized heavy chain variable domain

SEQ ID NO: 11 - protein, 113 aa, Artificial Sequence
  human light chain variable domain
DIVMTQSPDSLAVSLGERATINCKSSQSLWDSTNQKNFLTWYQQKPGQPPKLLIYWTSTRESGVPDRFSGSGSGTDFTLTISSLQAEDVAVYYCQNDYFYPLTFGGGTKVEIK

SEQ ID NO: 12 - protein, 117 aa, Artificial Sequence; identical to SEQ ID NO: 8
  human heavy chain variable domain

SEQ ID NO: 13 - protein, 112 aa, Artificial Sequence
  humanized light chain variable domain
DIVTQSPDSLAVSLGERATINCKSSQSLWDSGNQKNFLTWYQQKPGQPPKLLIYWTSTRESGVPDRFSGSGSGTDFTLTISSLQAEDVAVYYCQNDYFYPLTFGGGTKVEIK

SEQ ID NO: 14 - protein, 117 aa, Artificial Sequence
  humanized heavy chain variable domain
QVQLVWSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSS

SEQ ID NO: 15 - protein, 113 aa, Artificial Sequence; identical to SEQ ID NO: 7
  humanized light chain variable domain

SEQ ID NO: 16 - protein, 117 aa, Artificial Sequence
  humanized heavy chain variable domain
QVQLVQSGAEVKKPGASVKVSCKASGYTFTSYWINWVRQAPGQGLEQMGNIYPGSSLTNYNEKFKNRVTMTRDTSTSTVYMELSSLRSEDTAVYYCARLSTGTFAYWGQGTLCTVSS

SEQ ID NO: 17 - protein, 444 aa, Artificial Sequence
  humanized heavy chain
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - protein, 443 aa, Artificial Sequence
  humanized heavy chain
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 19 - protein, 221 aa, Artificial Sequence
  humanized light chain
DIVMTQSPDSLAVSLGERATINCKSSQSLWDSGNQKNFLTWYQQKPGQPPKLLIYWTSYRESGVPDRFSGSGSGTDFTLTISSLQAEDVAVYYCQNDYFYPHTFGGGTKVEIKRGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 117 aa, Artificial Sequence
  humanized light chain variable domain
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSS

SEQ ID NO: 21 - protein, 117 aa, Artificial Sequence
  humanized heavy chain variable domain
QVQLVQSGAEVKKPGASVKVSCKASGYTFTSYWINWVRQAPGQGLEWMGNIWPGSSLTNYNEKFKNRVTMTRDTSTSTVYMELSSLRSEDTAVYYCARLLTGTFAYWGQGTLVTVSS

SEQ ID NO: 22 - protein, 112 aa, Artificial Sequence
  humanized light chain variable domain
DIVTQSPDSLAVSLGERATINCKSSQSLWDSTNQKNFLTWYQQKPGQPPKLLIYWTSTRESGVPDRFSGSGSGTDFTLTISSLQAEDVAVYYCQNDYFYPLTFGGGTKVEIK

SEQ ID NO: 23 - protein, 116 aa, Artificial Sequence
  humanized heavy chain variable domain
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQTLVTVSS

SEQ ID NO: 24 - protein, 113 aa, Artificial Sequence; identical to SEQ ID NO: 5
  light chain variable domain

SEQ ID NO: 25 - protein, 117 aa, Artificial Sequence
  humanized heavy chain variable domain
QVQLVQSGAEVKKPGASVKVSCKASGYTFTSYWINWVRQAPGQGLEWMGNIYPGSSITNYNEKFKNRVTMTRDTSTSTVYMELSSLRSEDTAVYYCARLTTGTFAYWGQGTLVTVSS

SEQ ID NO: 26 - protein, 113 aa, Artificial Sequence; identical to SEQ ID NO: 7
  humanized light chain variable domain

SEQ ID NO: 27 - protein, 117 aa, Artificial Sequence; identical to SEQ ID NO: 25
  humanized heavy chain variable domain

SEQ ID NO: 28 - protein, 113 aa, Artificial Sequence; identical to SEQ ID NO: 9
  humanized light chain variable domain

SEQ ID NO: 29 - protein, 117 aa, Artificial Sequence; identical to SEQ ID NO: 25
  humanized heavy chain variable domain

SEQ ID NO: 30 - protein, 113 aa, Artificial Sequence; identical to SEQ ID NO: 11
  humanized light chain variable domain

SEQ ID NO: 31 - protein, 117 aa, Artificial Sequence; identical to SEQ ID NO: 25
  humanized heavy chain variable domain

SEQ ID NO: 32 - protein, 113 aa, Artificial Sequence; identical to SEQ ID NO: 5
  humanized light chain variable domain

SEQ ID NO: 33 - protein, 117 aa, Artificial Sequence; identical to SEQ ID NO: 21
  humanized heavy chain variable domain

SEQ ID NO: 34 - protein, 113 aa, Artificial Sequence; identical to SEQ ID NO: 7
  humanized light chain variable domain

SEQ ID NO: 35 - protein, 117 aa, Artificial Sequence; identical to SEQ ID NO: 21
  humanized heavy chain variable domain

SEQ ID NO: 36 - protein, 113 aa, Artificial Sequence; identical to SEQ ID NO: 9
  humanized light chain variable domain

SEQ ID NO: 37 - protein, 113 aa, Artificial Sequence; identical to SEQ ID NO: 11
  humanized light chain variable domain

SEQ ID NO: 38 - protein, 117 aa, Artificial Sequence; identical to SEQ ID NO: 21
  humanized heavy chain variable domain

SEQ ID NO: 39 - protein, 432 aa, Artificial Sequence
  Anti-PD-1 fusion protein AMP-224
LFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWEPKSCDKTHTCPPCPAPELLGGPSVFLFPPKPKDTLMISRTPEVTCWVDVSHEDPEVKFNWYVDGVEVHNAKTKPREEQYNSTYRWSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - DNA, 25 bp, Artificial Sequence
  PD-1 Binding antagonist Aptamer
gctactgtacatcacgcctctcccc

SEQ ID NO: 41 - DNA, 24 bp, Artificial Sequence
  PD-1 Binding antagonist Aptamer
ctactgtacatcacgcctctcccc

SEQ ID NO: 42 - DNA, 25 bp, Artificial Sequence
  PD-1 Binding antagonist Aptamer
gtacagttcccgtccctgcactaca

SEQ ID NO: 43 - DNA, 24 bp, Artificial Sequence
  PD-1 Binding antagonist Aptamer
gtacagttcccgtcctgcactaca

SEQ ID NO: 44 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 45 - DNA, 22 bp, Artificial Sequence
  synthetic oligonucleotide
aagctctttctttctctctgtt

SEQ ID NO: 46 - DNA, 28 bp, Artificial Sequence
  synthetic oligonucleotide
accgagctacttttccagaaggtatatt

SEQ ID NO: 47 - DNA, 20 bp, Artificial Sequence
  synthetic oligonucleotide
cccatgatagccgtctttaa

SEQ ID NO: 48 - DNA, 11 bp, Artificial Sequence
  synthetic oligonucleotide
ctttctctctg

SEQ ID NO: 49 - DNA, 19 bp, Artificial Sequence
  synthetic oligonucleotide
ttctctctgttttaagatc

SEQ ID NO: 50 - DNA, 12 bp, Artificial Sequence
  synthetic oligonucleotide
ctttctctctgt

(19) United States
(12) Patent Application Publication
(10) Pub. No.: US 2023/0117328 A1
(43) Pub. Date: Apr. 20, 2023

(54) METHODS TO TREAT CANCER USING (R)-N-(3-FLUORO-4-((3-((1-HYDROXYPROPAN-2-YL)AMINO)-1H-PYRAZOLO[3,4-B]PYRIDIN-4-YL)OXY)PHENYL)-3-(4-FLUOROPHENYL)-1-ISOPROPYL-2,4-DIOXO-1,2,3,4-TETRAHYDROPYRIMIDINE-5-CARBOXAMIDE

(71) Applicant: Array BioPharma Inc., Boulder, CO (US)

(72) Inventors: Karyn Sue BOUHANA, Westminster, CO (US); Jim Yuk-Fai WONG, Denver, CO (US)

(73) Assignee: Array BioPharma Inc., Boulder, CO (US)

(21) Appl. No.: 17/905,536

(22) PCT Filed: Mar. 1, 2021
(86) PCT No.: PCT/IB2021/051693

§ 371 Date: Sep. 2, 2022

Related U.S. Application Data
(60) Provisional application No. 63/133,501, filed on Jan. 4, 2021.
(60) Provisional application No. 62/984,458, filed on Mar. 3, 2020.

(51) Int. Cl. A61K 31/513; A61K 39/395; A61K 9/48; A61P 35/00

(52) U.S. Cl. CPC A61K 31/513; A61K 39/3955; A61K 9/4816; A61K 9/4858; A61K 9/4866; A61K 9/485; A61P 35/00

(57) ABSTRACT

This invention relates to a method of treating cancer by administering to a patient in need thereof, over a period of time, therapeutic agents that comprises Compound 1 or a pharmaceutically acceptable salt thereof on an intermittent dosing schedule alone or in combination with a PD-1 or PD-L1 inhibitor, to a patient in need thereof.

### CROSS-REFERENCE TO RELATED APPLICATIONS

This application claims the benefit of priority of U.S. Provisional Application No. 62/984,458 filed Mar. 3, 2020, and U.S. Provisional Application No. 63/133,501 filed Jan. 4, 2021, the contents of which are hereby incorporated by reference in their entireties.

### FIELD OF THE INVENTION

The present invention relates to methods useful for the treatment of proliferative diseases, including cancer which can be treated with a TAM kinase inhibitor and/or a MET kinase inhibitor. In particular, this invention relates to methods for treating cancer by administering (R)—N-(3-fluoro-4-((3-((1-hydroxypropan-2-yl)amino)-1H-pyrazolo[3,4-b]pyridin-4-yl)oxy)phenyl)-3-(4-fluorophenyl)-1-isopropyl-2,4-dioxo-1,2,3,4-tetrahydropyrimidine-5-carboxamide or a pharmaceutically acceptable salt thereof on an intermittent dosing schedule alone or in combination with a PD-1 or PD-L1 inhibitor.

### BACKGROUND

Receptor tyrosine kinases (RTKs) are cell surface proteins that transmit signals from the extracellular environment to the cell cytoplasm and nucleus to regulate cellular events such as survival, growth, proliferation, differentiation, adhesion and migration.

The TAM subfamily consists of three RTKs including TYRO3, AXL and Mer (Graham et al., 2014, Nature Reviews Cancer 14, 769-785; Linger et al., 2008, Advances in Cancer Research 100, 35-83). TAM kinases are characterized by an extracellular ligand binding domain consisting of two immunoglobulin-like domains and two fibronectin type III domains. Two ligands, growth arrest specific 6 (GAS6) and protein S (PROS1), have been identified for TAM kinases. GAS6 can bind to and activate all three TAM kinases, while PROS1 is a ligand for Mer and TYRO3 (Graham et al., 2014, Nature Reviews Cancer 14, 769-785).

AXL (also known as UFO, ARK, JTK11 and TYRO7) was originally identified as a transforming gene from DNA of patients with chronic myelogenous leukemia (O'Bryan et al., 1991, Mol Cell Biol 11, 5016-5031; Graham et al., 2014, Nature Reviews Cancer 14, 769-785; Linger et al., 2008, Advances in Cancer Research 100, 35-83). GAS6 binds to AXL and induces subsequent auto-phosphorylation and activation of AXL tyrosine kinase. AXL activates several downstream signaling pathways including PI3K-Akt, Raf-MAPK, PLC-PKC (Feneyrolles et al., 2014, Molecular Cancer Therapeutics 13, 2141-2148; Linger et al., 2008, Advances in Cancer Research 100, 35-83).

MER (also known as MERTK, EYK, RYK, RP38, NYK and TYRO 12) was originally identified as a phospho-protein from a lymphoblastoid expression library (Graham et al., 1995, Oncogene 10, 2349-2359; Graham et al., 2014, Nature Reviews Cancer 14, 769-785; Linger et al., 2008, Advances in Cancer Research 100, 35-83). Both GAS6 and PROSI can bind to Mer and induce the phosphorylation and activation of Mer kinase (Lew et al., 2014). Like AXL, MER activation also conveys downstream signaling pathways including PI3K-Akt and Raf-MAPK (Linger et al., 2008, Advances in Cancer Research 100, 35-83).

TYRO3 (also known as DTK, SKY, RSE, BRT, TIF, ETK2) was originally identified through a PCR-based cloning study (Lai et al., Neuron 6, 691-70, 1991; Graham et al., 2014, Nature Reviews Cancer 14, 769-785; Linger et al., 2008, Advances in Cancer Research 100, 35-83). Both ligands, GAS6 and PROS1, can bind to and activate TYRO3. Although the signaling pathways downstream of TYRO3 activation are the least studied among TAM RTKs, it appears that both PI3K-Akt and Raf-MAPK pathways are involved (Linger et al., 2008, Advances in Cancer Research 100, 35-83). AXL, MER and TYRO3 are found to be over-expressed in cancer cells.

The MET family includes mesenchymal-epithelial transition factor (c-Met), a single pass tyrosine kinase receptor that is expressed on the surface of various epithelial cells; its ligand is hepatocyte growth factor/scatter factor (HGF/SF) (Nakamura et al., Nature 342:440-443, 1989). The binding of HFG to c-Met initiates a series of intracellular signals that mediate embryogenesis and would healing in normal cells (Organ, Ther. Adv. Med. Oncol. 3(1 Supply):S7-S19, 2011). However, in cancer cells, aberrant HGF/c-Met axis activation, which is closely related to c-Met gene mutations, overexpression, and amplification, promotes tumor development and progression—e.g., by stimulating the PI3K/AKT, Ras/MAPK, JAK/STAT, SRC, and Wnt/β-catenin signal pathways (Zhang et al., Mol. Cancer 17:45, 2018; Mizuno et al., Int. J. Mol. Sci. 14:888-919, 2013). The constitutive activation of the aforementioned c-Met-dependent signaling pathways confers cancer cells with competitive growth advantage relative to normal cells and increases the likelihood of metastasis—e.g., by enabling access to blood supply and conferring ability to dissociate from tissues (Comoglio et al., Nat. Rev. Drug Discov 7:504-516, 2008; Birchmeier et al., Nat. Rev. Mol. Cell. Biol. 4:915-925, 2003).

PD-L1 is overexpressed in many cancers and is often associated with poor prognosis (Okazaki T et al., Intern. Immun. 2007 19(7):813) (Thompson R H et al., Cancer Res 2006, 66(7):3381). Antibodies targeting the PD-1/PD-L1 pathway act by reactivating anti-tumor CD8 T cells that have been rendered nonfunctional (exhausted) by expression of PD-L1 within the tumor microenvironment. However, the efficacy and durability of this approach is limited by the ability of the immune system to generate tumor specific T cells (T cell priming) and by immune suppressive myeloid cells present within the tumor.

There remains a need for advantageous therapies, including combination therapies, for treating cancer patients, or particular populations of cancer patients, and potentially with particularized dosing schedules to optionally improve the safety profile.

### SUMMARY

In one embodiment, provided herein is a method for treating cancer comprising administering to a patient in need thereof, over a period of time, a therapeutically effective amount of (R)—N-(3-fluoro-4-((3-((1-hydroxypropan-2-yl)amino)-1H-pyrazolo[3,4-b]pyridin-4-yl)oxy)phenyl)-3-(4-fluorophenyl)-1-isopropyl-2,4-dioxo-1,2,3,4-tetrahydropyrimidine-5-carboxamide (hereinafter “Compound 1”) or a pharmaceutically acceptable salt thereof as a monotherapy, wherein Compound 1 or a pharmaceutically acceptable salt thereof is administered according to an intermittent dosing schedule of at least one dosing cycle, wherein each dosing cycle comprises (a) a dosing period during which said Compound 1 or a pharmaceutically acceptable salt thereof is administered, and (b) a resting period during which said Compound 1 or a pharmaceutically acceptable salt thereof is not administered. In one embodiment, Compound 1 is administered as the free base. In one embodiment, Compound 1 is administered as a hydrochloride salt (hereinafter “Compound 1 HCl”).

In one embodiment, provided herein is a method for treating cancer comprising administering to a patient in need thereof, over a period of time, a therapeutically effective amount of Compound 1 or a pharmaceutically acceptable salt thereof and a PD-1 inhibitor according to an intermittent dosing schedule of at least one dosing cycle, wherein each dosing cycle comprises (a) a dosing period during which said Compound 1 or a pharmaceutically acceptable salt thereof and said PD-1 inhibitor are administered, and (b) a resting period during which said Compound 1 or a pharmaceutically acceptable salt thereof and said PD-1 inhibitor are not administered. In one embodiment, Compound 1 is administered as the free base. In one embodiment, Compound 1 is administered as Compound 1 HCl.

In one embodiment, provided herein is a method for treating cancer comprising administering to a patient in need thereof, over a period of time, a therapeutically effective amount of Compound 1 or a pharmaceutically acceptable salt thereof and a PD-L1 inhibitor according to an intermittent dosing schedule of at least one dosing cycle, wherein each dosing cycle comprises (a) a dosing period during which said Compound 1 or a pharmaceutically acceptable salt thereof and said PD-L1 inhibitor are administered, and (b) a resting period during which said Compound 1 or a pharmaceutically acceptable salt thereof and said PD-L1 inhibitor are not administered. In one embodiment, Compound 1 is administered as the free base. In one embodiment, Compound 1 is administered as Compound 1 HCl.

In some embodiments of any of the methods described herein, the patient was previously treated with at least one anticancer therapy or agent (e.g., any of the anticancer agents described herein) prior to the first dosing cycle (i.e., prior to the “period of time”). In one embodiment, the previous treatment with the at least one anticancer agent was unsuccessful (e.g., the patient previously developed resistance to one or more of the at least one additional anticancer agent).

In some embodiments of any of the methods described herein, the patient is naïve to treatment with another anticancer therapy or agent prior to the first dosing period (i.e., prior to said period of time).

In one embodiment, provided herein is a method for reducing ocular toxicity due to administration of Compound 1 or a pharmaceutically acceptable salt thereof to a patient having cancer by administering Compound 1 or a pharmaceutically acceptable salt thereof as a monotherapy according to an intermittent dosing schedule.

In one embodiment, provided herein is a method for reducing ocular toxicity due to administration of Compound 1 or a pharmaceutically acceptable salt thereof to a patient having cancer by administering Compound 1 or a pharmaceutically acceptable salt thereof according to an intermittent dosing schedule in combination with a PD-1 inhibitor or a PD-L1 inhibitor.

### BRIEF DESCRIPTION OF THE FIGURES

FIG. 1 is a graph showing tumor volume following administration of Compound 1 alone or in combination with an anti-PD-1 antibody.

FIG. 2 is a XRPD scan of a spray-dried dispersion comprising Compound 1 HCl:HMPCAS-MG, 25%:75% w/w.

### DETAILED DESCRIPTION

Dosing schedules of Compound 1 or a pharmaceutically acceptable salt thereof have been discovered which allow the administration of a dose which ensures efficacy in treating cancer but at the same time reduces the risk of the occurrence of certain adverse events, for example ocular toxicity.

In one embodiment, provided herein is a method for treating cancer comprising administering to a patient in need thereof, over a period of time, a therapeutically effective amount of Compound 1 or a pharmaceutically acceptable salt thereof as a monotherapy, wherein Compound 1 or a pharmaceutically acceptable salt thereof is administered according to an intermittent dosing schedule of at least one dosing cycle, wherein each dosing cycle comprises (a) a dosing period during which Compound 1 or a pharmaceutically acceptable salt thereof is administered, and (b) a resting period during which Compound 1 or a pharmaceutically acceptable salt thereof is not administered. In one embodiment, Compound 1 is administered as the free base. In one embodiment, Compound 1 is administered as Compound 1-HCl.

Compound 1 is known by the chemical name (R)—N-(3-fluoro-4-((3-((1-hydroxypropan-2-yl)amino)-1H-pyrazolo[3,4-b]pyridin-4-yl)oxy)phenyl)-3-(4-fluorophenyl)-1-isopropyl-2,4-dioxo-1,2,3,4-tetrahydropyrimidine-5-carboxamide and has the following structure:

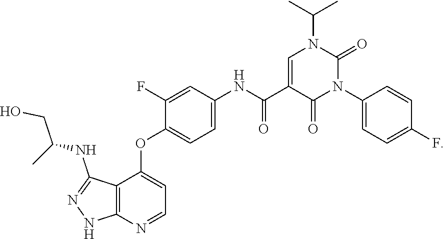 [SMILES: CC(C)n1cc(C(=O)Nc2ccc(Oc3ccnc4[nH]nc(N[C@H](C)CO)c34)c(F)c2)c(=O)n(-c2ccc(F)cc2)c1=O]

Methods of preparing Compound 1 and its pharmaceutically acceptable salts are described in PCT/US2019/048701, which published Mar. 5, 2020 as WO 2020/047184 A1, the disclosure of which is incorporated by reference herein in its entirety. In one embodiment, Compound 1 is a free base. In one embodiment, Compound 1 is in the form of a pharmaceutically acceptable salt. In one embodiment, Compound 1 is a Compound 1 HCl.

In one embodiment, provided herein is a method for treating cancer comprising administering to a patient in need thereof, over a period of time, a therapeutically effective amount of Compound 1 or a pharmaceutically acceptable salt thereof and a PD-1 inhibitor according to an intermittent dosing schedule of at least one dosing cycle, wherein each dosing cycle comprises (a) a dosing period during which said Compound 1 or a pharmaceutically acceptable salt thereof and said PD-1 inhibitor are administered, and (b) a resting period during which said Compound 1 or a pharmaceutically acceptable salt thereof and said PD-1 inhibitor are not administered. In one embodiment, Compound 1 is administered as the free base. In one embodiment, Compound 1 is administered as Compound 1 HCl.

In one embodiment, provided herein is a method for treating cancer comprising administering to a patient in need thereof, over a period of time, a therapeutically effective amount of Compound 1 or a pharmaceutically acceptable salt thereof and a PD-L1 inhibitor according to an intermittent dosing schedule of at least one dosing cycle, wherein each dosing cycle comprises (a) a dosing period during which said Compound 1 or a pharmaceutically acceptable salt thereof and said PD-L1 inhibitor are administered, and (b) a resting period during which said Compound 1 or a pharmaceutically acceptable salt thereof and said PD-L1 inhibitor are not administered. In one embodiment, Compound 1 is administered as the free base. In one embodiment, Compound 1 is administered as Compound 1 HCl.

It has also been discovered that use of certain intermittent dosing schedules for the administration of Compound 1 or a pharmaceutically acceptable salt thereof for the treatment of cancer reduces ocular toxicity that may result due to administration of Compound 1 or a pharmaceutically acceptable salt thereof. In particular, it was discovered that intermittent dosing of Compound 1 or a pharmaceutically acceptable salt thereof can eliminate and/or decrease the incidence and severity of retinal toxicity with no negative impact on anti-tumor efficacy. In one embodiment, Compound 1 is administered as the free base. In one embodiment, Compound 1 is administered as Compound 1 HCl.

In one embodiment, provided herein is a method for reducing ocular toxicity due to administration of Compound 1 or a pharmaceutically acceptable salt thereof to a patient having cancer by administering Compound 1 or a pharmaceutically acceptable salt thereof to said patient as a monotherapy according to an intermittent dosing schedule. In one embodiment, Compound 1 is administered as the free base. In one embodiment, Compound 1 is administered as Compound 1 HCl.

In one embodiment, provided herein is a method for reducing ocular toxicity due to administration of Compound 1 or a pharmaceutically acceptable salt thereof to a patient having cancer by administering Compound 1 or a pharmaceutically acceptable salt thereof according to an intermittent dosing schedule in combination with a PD-1 inhibitor.

In one embodiment, provided herein is a method for reducing ocular toxicity due to administration of Compound 1 or a pharmaceutically acceptable salt thereof to a patient having cancer by administering Compound 1 or a pharmaceutically acceptable salt thereof according to an intermittent dosing schedule in combination with a PD-L1 inhibitor.

### General Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

“About” when used to modify a numerically defined parameter (e.g., the dose of Compound 1 or a pharmaceutically acceptable salt thereof, or a PD-1 inhibitor, or a PD-L1 inhibitor, or the length of treatment time with a combination therapy described herein) means that the parameter may vary by as much as 10% below or above the stated numerical value for that parameter. For example, a dose of about 5 mg/kg may vary between 4.5 mg/kg and 5.5 mg/kg. “About” when used at the beginning of a listing of parameters is meant to modify each parameter. For example, about 0.5 mg, 0.75 mg or 1.0 mg means about 0.5 mg, about 0.75 mg or about 1.0 mg. Likewise, about 5% or more, 10% or more, 15% or more, 20% or more, and 25% or more means about 5% or more, about 10% or more, about 15% or more, about 20% or more, and about 25% or more.

The term “dosing schedule” refers to the dose and timing of administration of each therapeutic agent.

The term “intermittent dosing schedule” refers to repeating dosing cycles of administration of one or more drugs in which the one or more drugs is administered on one or more consecutive days (“dosing period”) followed by one or more consecutive days of rest on which the one or more drugs is not administered (“resting period”). The cycles may be regular, in that the pattern of days on and days off is the same in each cycle, or the cycles may be irregular.

The term “dosing cycle” indicates the number and order of days which form one treatment scheme comprising administration (dosing) days and resting days before this treatment scheme is then repeated again.

“Dosing period” as used herein refers to a period of one or more days on which the patient is administered Compound 1 or a pharmaceutically acceptable salt thereof, alone or in combination with a PD-1 inhibitor or a PD-L1 inhibitor. The patient may take Compound 1 or a pharmaceutically acceptable salt thereof as one single dose on that day or the daily dose may be split up into smaller portions, e.g. in the morning one half of the daily dose and in the evening the other half.

“Resting period” as used herein refers to those days during which the patient is not administered Compound 1 or a pharmaceutically acceptable salt thereof when Compound 1 or a pharmaceutically acceptable salt thereof is administered alone, or those days during which the patient is not administered Compound 1 or a pharmaceutically acceptable salt thereof or a PD-1 inhibitor or a PD-L1 inhibitor when the dosing schedule includes administration of Compound 1 or a pharmaceutically acceptable salt thereof in combination with a PD-1 or PD-L1 inhibitor, respectively.

The phrase “period of time” refers to one or more dosing cycles during which a patient is treated with Compound 1 or a pharmaceutically acceptable salt thereof alone or in combination with a PD-1 or PD-L1 inhibitor.

The phrases “prior to a period of time” or “before a period of time” refer to (1) the completion of administration of surgery and/or radiation treatment to the subject before the first administration of Compound 1 or a pharmaceutically acceptable salt thereof alone or in combination with a PD-1 or PD-L1 inhibitor according to a method described herein, and/or (2) the administration of one or more therapeutic agents (e.g., one or more anticancer agents other than Compound 1 or a pharmaceutically acceptable salt thereof alone) to the subject before a first administration of Compound 1 or a pharmaceutically acceptable salt thereof alone or in combination with a PD-1 or PD-L1 inhibitor. In one embodiment, the one or more previously administered therapeutic agents are present in subtherapeutic and/or undetectable levels in the subject at the time the first administration of Compound 1 or a pharmaceutically acceptable salt thereof alone or in combination with a PD-1 or PD-L1 inhibitor according to a method described herein is performed.

The phrase “pharmaceutically acceptable” indicates that the substance or composition must be compatible chemically and/or toxicologically, with the other ingredients comprising a formulation, and/or the mammal being treated therewith. Some embodiments relate to the pharmaceutically acceptable salts of the compounds described herein. In certain instances, pharmaceutically acceptable salts are obtained by reacting Compound 1 with acids such as hydrochloric acid.

“Administration”, “administering”, “treating”, and “treatment,” as it applies to a patient, individual, animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell.

“Treatment” and “treating”, as used in a clinical setting, is intended for obtaining beneficial or desired clinical results. For purposes of this invention, beneficial or desired clinical results include, but are not limited to, one or more of the following: reducing the proliferation of (or destroying) neoplastic or cancerous cells, inhibiting metastasis of neoplastic cells, shrinking or decreasing the size of a tumor, remission of a disease (e.g., cancer), decreasing symptoms resulting from a disease (e.g., cancer), increasing the quality of life of those suffering from a disease (e.g., cancer) (e.g., assessed using FACT-G or EORTC-QLQC30), decreasing the dose of other medications required to treat a disease (e.g., cancer), delaying the progression of a disease (e.g., cancer), and/or prolonging survival of patients having a disease (e.g., cancer). For example, treatment can be the diminishment of one or several symptoms of a disorder, such as cancer. Within the meaning of the present invention, the term “treat” also denotes to arrest, delay the onset (i.e., the period prior to clinical manifestation of a disease) and/or reduce the risk of developing or worsening a disease. “Treatment” can also mean prolonging survival as compared to expected survival if not receiving treatment, for example, an increase in overall survival (OS) compared to a subject not receiving treatment as described herein, and/or an increase in progression-free survival (PFS) compared to a subject not receiving treatment as described herein. The term “treating” can also mean an improvement in the condition of a subject having a cancer, e.g., one or more of a decrease in the size of one or more tumor(s) in a subject, a decrease or no substantial change in the growth rate of one or more tumor(s) in a subject, a decrease in metastasis in a subject, and an increase in the period of remission for a subject (e.g., as compared to the one or more metric(s) in a subject having a similar cancer receiving no treatment or a different treatment, or as compared to the one or more metric(s) in the same subject prior to treatment). As used herein, the terms “treating” and “treating” when referring, e.g., to the treatment of a cancer, are not intended to be absolute terms. For example, “treatment of cancer” and “treating cancer”, as used in a clinical setting, is intended to include obtaining beneficial or desired clinical results and can include an improvement in the condition of a subject having cancer. Beneficial or desired clinical results include, but are not limited to, one or more of the following: reducing the proliferation of (or destroying) neoplastic or cancerous cells, inhibiting metastasis of neoplastic cells, a decrease in metastasis in a subject, shrinking or decreasing the size of a tumor, change in the growth rate of one or more tumor(s) in a subject, an increase in the period of remission for a subject (e.g., as compared to the one or more metric(s) in a subject having a similar cancer receiving no treatment or a different treatment, or as compared to the one or more metric(s) in the same subject prior to treatment), decreasing symptoms resulting from a disease, increasing the quality of life of those suffering from a disease (e.g., assessed using FACT-G or EORTC-QLQC30), decreasing the dose of other medications required to treat a disease, delaying the progression of a disease, and/or prolonging survival of subjects having a disease. “Treatment” can also mean prolonging survival as compared to expected survival if not receiving treatment, for example, an increase in overall survival (OS) compared to a subject not receiving treatment as described herein, and/or an increase in progression-free survival (PFS) compared to a subject not receiving treatment as described herein.

The term “subject” includes any organism, preferably an animal, more preferably a mammal (e.g., rat, mouse, dog, cat, and rabbit) and most preferably a human.

A “patient” to be treated according to this invention includes any warm-blooded animal, such as, but not limited to human, monkey or other lower-order primate, horse, dog, rabbit, guinea pig, or mouse. In one embodiment the patient is a human. In one embodiment, the patient is a pediatric patient. Those skilled in the medical art are readily able to identify individuals who are afflicted with cancer and who are in need of treatment.

The term “pediatric patient” as used herein refers to a patient under the age of 16 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994.

“Ameliorating” means a lessening or improvement of one or more symptoms as compared to not administering a treatment. “Ameliorating” also includes shortening or reduction in duration of a symptom.

The term “regulatory agency” is a country's agency for the approval of the medical use of pharmaceutical agents with the country. For example, a non-limiting example of a regulatory agency is the U.S. Food and Drug Administration (FDA).

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also antigen binding fragments thereof (such as Fab, Fab′, F (ab′)₂, Fv), single chain (scFv) and domain antibodies (including, for example, shark and camelid antibodies), and fusion proteins comprising an antibody, and any other modified configuration of the immunoglobulin molecule that comprises an antigen recognition site. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

The term “antigen binding fragment” or “antigen binding portion” of an antibody, as used herein, refers to one or more fragments of an intact antibody that retain the ability to specifically bind to a given antigen (e.g., PD-1). Antigen binding functions of an antibody can be performed by fragments of an intact antibody. Examples of binding fragments encompassed within the term “antigen binding fragment” of an antibody include Fab; Fab′; F (ab′) 2; an Fd fragment consisting of the VH and CH1 domains; an Fv fragment consisting of the VL and VH domains of a single arm of an antibody; a single domain antibody (dAb) fragment (Ward et al., Nature 341:544-546, 1989), and an isolated complementarity determining region (CDR).

An antibody, an antibody conjugate, or a polypeptide that “preferentially binds” or “specifically binds” (used interchangeably herein) to a target (e.g., PD-1 protein) is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a PD-1 epitope is an antibody that binds this epitope with greater affinity, avidity, more readily, and/or with greater duration than it binds to other PD-1 epitopes or non-PD-1 epitopes. It is also understood that by reading this definition, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

A “variable region” of an antibody refers to the variable region of the antibody light chain or the variable region of the antibody heavy chain, either alone or in combination. As known in the art, the variable regions of the heavy and light chain each consist of four framework regions (FR) connected by three complementarity-determining regions (CDRs) also known as hypervariable regions. The CDRs in each chain are held together in close proximity by the FRs and, with the CDRs from the other chain, contribute to the formation of the antigen binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Al-lazikani et al., 1997, J. Molec. Biol. 273:927-948). As used herein, a CDR may refer to CDRs defined by either approach or by a combination of both approaches.

A “CDR” of a variable domain are amino acid residues within the variable region that are identified in accordance with the definitions of the Kabat, Chothia, the accumulation of both Kabat and Chothia, AbM, contact, and/or conformational definitions or any method of CDR determination well known in the art. Antibody CDRs may be identified as the hypervariable regions originally defined by Kabat et al. See, e.g., Kabat et al., 1992, Sequences of Proteins of Immunological Interest, 5th ed., Public Health Service, NIH, Washington D.C. The positions of the CDRs may also be identified as the structural loop structures originally described by Chothia and others. See, e.g., Chothia et al., Nature 342:877-883, 1989. Other approaches to CDR identification include the “AbM definition,” which is a compromise between Kabat and Chothia and is derived using Oxford Molecular's AbM antibody modeling software (now Accelrys®), or the “contact definition” of CDRs based on observed antigen contacts, set forth in MacCallum et al., J. Mol. Biol., 262:732-745, 1996. In another approach, referred to herein as the “conformational definition” of CDRs, the positions of the CDRs may be identified as the residues that make enthalpic contributions to antigen binding. See, e.g., Makabe et al., Journal of Biological Chemistry, 283:1156-1166, 2008. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues or even entire CDRs do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, AbM, contact, and/or conformational definitions.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by the hybridoma method first described by Kohler et al. (Nature (1975) 256: 495), or may be made by recombinant DNA methods (see, e.g., U.S. Pat. No. 4,816,567). The “monoclonal antibodies” may also be isolated from phage antibody libraries using the techniques described in Clackson et al. Nature 352: 624-628 (1991)) and Marks et al. (J. Mol. Biol. 222: 581-597 (1991)), for example. See also Presta (J. Allergy Clin. Immunol. 116:731 (2005)).

“Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table A below.

TABLE A
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

The term “PD-1 inhibitor” as used herein refers to a molecule that binds specifically to PD-1 and decreases the interaction of PD-1 with one or more of its binding partners, such as PD-L1 and/or PD-L2. For example, PD-1 inhibitors include anti-PD-1 antibodies, antigen binding fragments thereof, immunoadhesins, aptamers, fusion proteins, and oligopeptides. In one embodiment, a PD-1 inhibitor reduces the negative co-stimulatory signal mediated by or through cell surface proteins expressed on T lymphocytes mediated signaling through PD-1 so as render a dysfunctional T-cell less dysfunctional. In some embodiments, the PD-1 inhibitor is an anti-PD-1 antibody. In some embodiments, the PD-1 inhibitor is pembrolizumab (Keytruda®), a biosimilar of pembrolizumab nivolumab (Opdivo®), a biosimilar of nivolumab, cemiplimab (Libtayo®), pidilizumab, or 1141PDCA-170. In one embodiment, the PD-1 inhibitor is an anti-PD-1 antibody selected from nivolumab, pembrolizumab, sasanlimab, a biosimilar of nivolumab, a biosimilar of pembrolizumab and a biosimilar of sasanlimab. In one embodiment, the PD-1 inhibitor is nivolumab or a biosimilar thereof. In one embodiment, the PD-1 inhibitor is pembrolizumab or a biosimilar thereof. In one embodiment, the PD-1 inhibitor is sasanlimab or a biosimilar thereof.

An anti-PD-1 antibody as described herein can also be an antigen-binding antibody fragment of nivolumab or a biosimilar thereof, or an antigen-binding antibody fragment of pembrolizumab or a biosimilar thereof, or an antigen-binding antibody fragment of sasanlimab (also known as RN888) or a biosimilar thereof. In some embodiments, an anti-PD-1 antibody can be a biosimilar of nivolumab or a biosimilar of pembrolizumab or a biosimilar of sasanlimab.

Table B below provides a list of the amino acid sequences of exemplary PD-1 inhibitors for use in the treatment method, medicaments and uses of the present invention. CDRs are underlined for mAb7 and mAb15. The mAB7 is also known as RN888 or PF-6801591. mAb7 (aka RN888) and mAb15 are disclosed in International Patent Publication No. WO2016/092419, the disclosure of which is hereby incorporated by reference in its entirety.

TABLE B

| | |
|---|---|
| Nivolumab, MDX1106, full | QVQLVESGGGWQPGRSLRLDCKASGITFSNSGMHWVRQAPGKGLEWVA |
| length heavy chain | VrWYDGSKRYYADSVKGRFTISRDNSKNTLFLQMNSLRAEDTAVYYCA |
| From WO 2006/121168 | TNDDYWGQGTLVTVSSASTKGPSVFPLAPCSRSTSESTAALGCLVDYF |
| | PEPVTVSWNSGALTSGVHTFPAVLQSSGLYSLSSVVTVPSSSLGTTYT |
| | CNVDHKPSNTKVDRVESYGPPCPPCPAPEFLGGPSVFLFPPKPKDTLM |
| | ISRTPEVTCWVDVSQEDPEVQFNWYYDGVEVHNATKPREEQFNSTYRV |
| | VSVLTVLHQDWLNGKEYKCKVSNKGLPSSIEKTISKAGQPREPQVYTL |
| | PPSQEEMTKNQVSLTCLVKGFYPSDIAVEWESNGQPEKNYKTTPPVLD |
| | SDGSFFLYSRLTVDKSRWQEGNVFSCSVMHEALHNHYTQKSLSLSLGK |
| | (SEQ ID NO: 1) |
| Nivolumab, MDX1106, full | EIVLTQSPATLSLSPGERATLSCRASQSVSSYLAWYQQPGQAPRLLIY |
| length light chain | DASNRATGIPARFSGSGSGTDFTLTISSLEPEDFAVYYCQQSSNWPRT |
| From WO 2006/121168 | FGQGTKVEIRTVAAPSVFIFPPSDEQLSGTASVVCLLNNFYPREAVQW |
| | KVDNALQSGNSQESVTEQDSDSTYSLSSTLTLSKADYEKHKVYACEVT |
| | HQGLSSPVTSFNRGEC (SEQ ID NO: 2) |
| Pembrolizumab, MK3475, | QVQLVQSGVEVKKPGASVKVSCKASGYTFTNYYMYWVRQAPGQGLEWM |
| full length heavy chain | GGINPSNGGTNFNEKFKNRVTLTTDSSTTTAYMELKSLQFDDTAVYYC |
| From WO 2009/114335 | ARRDYRFDMGFDYWGQGTTVTVSSASTKGPSVFPLAPCSRSTSESTAA |
| | LGCLVKDYFPEPVTVSWNSGALTSGVHTFPAVLQSSGLYSLSSVVTVP |
| | SSSLGTKTYTCNVDHKPSNTKVDKRVESKYGPPCPPCPAPEFLGGPSV |
| | FLFPPKPKDTLMISRTPEVTCVVVDVSQEDPEVQFNWYVDGVEVHNAK |
| | TKPREEQFNSTYRVVSVLTVLHQDWLNGKEYKCKVSNKGLPSSIEKTI |
| | SKAKGQPREPQVYTLPPSQEEMTKNQVSLTCLVKGFYPSDIAVEWESN |
| | GQPENNYKTTPPVLDSDGSFFLYSRLTVDKSRWQEGNVFSCSVMHEAL |
| | HNHYTQKSLSLSLGK (SEQ ID NO: 3) |
| Pembrolizumab, MK3475, | EIVLTQSPATLSLSPGERATLSCRASKGVSTSGYSYLHWYQQKPGQAP |
| full length light chain | RLLIYLASYLESGVPARFSGSGSGTDFTLTISSLEPEDFAVYYCQHSR |
| From WO 2009/114335 | DLPLTFGGGTKVEIKRTVAAPSVFIFPPSDEQLKSGTASVVCLLNNFY |
| | PREAKVQWKVDNALQSGNSQESVTEQDSKDSTYSLSSTLTLSKADYEK |
| | HKVYACEVTHQGLSSPVTKSFNRGEC (SEQ ID NO: 4) |
| mAb1 light chain variable | DIVMTQSPDSLAVSLGERATINCKSSQSLWDSGNQKNFLTWYQQKPGQ |
| domain with CDRs in bold | PPKLLIYWTSTRESGVPDRFSGSGSGTDFTLTISSLQAEDVAVYYCQN |
| (from WO 16/92419) | DYFYPLTFGGGTKVEIK (SEQ ID NO: 5) |
| mAb1 heavy chain variable | QVQLVQSGAEVKKPGASVKVSCKASGYTFTSYWINWVRQAPGQGLEQM |
| domain with CDRs underlined | GNIYPGSSLTNYNEKFKNRVTMTRDTSTSTVYMELSSLRSEDTAVYYC |
| (from WO 16/92419) | ARLLTGTFAYWGQGTLVTVSS (SEQ ID NO: 6) |
| mAb2 light chain variable | DIVMTQSPDSLAVSLGERATINCKSSQSLWDSGNQKNFLTWYQQKPGQ |
| domain with CDRs underlined | PPKLLIYWTSYRESGVPDRFSGSGSGTDFTLTISSLQAEDVAVYYCQN |
| (from WO 16/92419) | DYFYPLTFGGGTKVEIK (SEQ ID NO: 7) |
| mAb2 heavy chain variable | QVQLVQSGAEVKKPGASVKVSCKASGYTFTSYWINWVRQAPGQGLEWM |
| domain with CDRs underlined | GNIYPGSSLTNYNEKFKNRVTMTRDTSTSTVYMELSSLRSEDTAVYYC |
| (from WO 16/92419) | ARLLTGTFAYWGQGTLVTVSS (SEQ ID NO: 8) |
| mAb3 light chain variable | DIVMTQSPDSLAVSLGERATINCKSSQSLWDSGNQKNFLTWYQQKPGQ |
| domain with CDRs underlined | PPKLLIYWTSYRESGVPDRFSGSGSGTDFTLTISSLQAEDVAVYYCQN |
| (from WO 16/92419) | DYFYPHTFGGGTKVEIK (SEQ ID NO: 9) |
| mAb3 heavy chain variable | QVQLVQSGAEVKKPGASVKVSCKASGYTFTSYWINWVRQAPGQGLEWM |
| domain with CDRs underlined | GNIYPGSSLTNYNEKFKNRVTMTRDTSTSTVYMELSSLRSEDTAVYYC |
| (from WO 16/92419) | ARLLTGTFAYWGQGTLVTVSS (SEQ ID NO: 10) |
| mAb4 light chain variable | DIVMTQSPDSLAVSLGERATINCKSSQSLWDSTNQKNFLTWYQQKPGQ |
| domain with CDRs underlined | PPKLLIYWTSTRESGVPDRFSGSGSGTDFTLTISSLQAEDVAVYYCQN |
| (from WO 16/92419) | DYFYPLTFGGGTKVEIK (SEQ ID NO: 11) |
| mAb4 heavy chain variable | QVQLVQSGAEVKKPGASVKVSCKASGYTFTSYWINWVRQAPGQGLEWM |
| domain with CDRs underlined | GNIYPGSSLTNYNEKFKNRVTMTRDTSTSTVYMELSSLRSEDTAVYYC |
| (from WO 16/92419) | ARLLTGTFAYWGQGTLVTVSS (SEQ ID NO: 12) |
| mAb5 light chain variable | DIVTQSPDSLAVSLGERATINCKSSQSLWDSGNQKNFLTWYQQKPGQP |
| domain with CDRs underlined | PKLLIYWTSTRESGVPDRFSGSGSGTDFTLTISSLQAEDVAVYYCQND |
| (from WO 16/92419) | YFYPLTFGGGTKVEIK (SEQ ID NO: 13) |
| mAb5 heavy chain variable | QVQLVWSGAEVKKPGASVKVSCKASGYTFTSYWINWVRQAPGQGLEWM |
| domain with CDRs underlined | GNIYPGSSLTNYNEKFKNRVTMTRDTSTSTVYMELSSLRSEDTAVYYC |
| (from WO 16/92419) | ARLSTGTFAYWGQGTLVTVSS (SEQ ID NO: 14) |
| mAb6 light chain variable | DIVMTQSPDSLAVSLGERATINCKSSQSLWDSGNQKNFLTWYQQKPGQ |
| domain with CDRs underlined | PPKLLIYWTSYRESGVPDRFSGSGSGTDFTLTISSLQAEDVAVYYCQN |
| (from WO 16/92419) | DYFYPLTFGGGTKVEIK (SEQ ID NO: 15) |
| mAb6 heavy chain variable | QVQLVQSGAEVKKPGASVKVSCKASGYTFTSYWINWVRQAPGQGLEQM |
| domain with CDRs underlined | GNIYPGSSLTNYNEKFKNRVTMTRDTSTSTVYMELSSLRSEDTAVYYC |
| (from WO 16/92419) | ARLSTGTFAYWGQGTLCTVSS (SEQ ID NO: 16) |
| mAb7(also known as | QVQLVQSGAEVKKPGASVKVSCKASGYTFTSYWINWVRQAPGQGLEWM |
| RN888) or mAb15 full- | GNIYPGSSLTNYNEKFKNRVTMTRDTSTSTVYMELSSLRSEDTAVYYC |
| length heavy chain | ARLSTGTFAYWGQGTLVTVSSASTKGPSVFPLAPCSRSTSESTAALGC |
| | LVKDYFPEPVTVSWNSGALTSGVHTFPAVLQSSGLYSLSSVVTVPSSS |
| | LGTKTYTCNVDHKPSNTKVDKRVESKYGPPCPPCPAPEFLGGPSVFLF |
| | PPKPKDTLMISRTPEVTCVVVDVSQEDPEVQFNWYVDGVEVHNAKTKP |
| | REEQFNSTYRVVSVLTVLHQDWLNGKEYKCKVSNKGLPSSIEKTISKA |
| | KGQPREPQVYTLPPSQEEMTKNQVSLTCLVKGFYPSDIAVEWESNGQP |
| | ENNYKTTPPVLDSDGSFFLYSRLTVDKSRWQEGNVFSCSVMHEALHNH |
| | YTQKSLSLSLGK (SEQ ID NO: 17) |
| mAb7 or mAb 15 full-length | QVQLVQSGAEVKKPGASVKVSCKASGYTFTSYWINWVRQAPGOGLEWM |
| heavy chain without the C- | GNIYPGSSLTNYNEKFKNRVTMTRDTSTSTVYMELSSLRSEDTAVYYC |
| terminal lysine | ARLSTGTFAYWGQGTLVTVSSASTKGPSVFPLAPCSRSTSESTAALGC |
| | LVKDYFPEPVTVSWNSGALTSGVHTFPAVLQSSGLYSLSSVVTVPSSS |
| | LGTKTYTCNVDHKPSNTKVDKRVESKYGPPCPPCPAPEFLGGPSVFLF |
| | PPKPKDTLMISRTPEVTCVVVDVSQEDPEVQFNWYVDGVEVHNAKTKP |
| | REEQFNSTYRVVSVLTVLHQDWLNGKEYKCKVSNKGLPSSIEKTISKA |
| | KGQPREPQVYTLPPSQEEMTKNQVSLTCLVKGFYPSDIAVEWESNGQP |
| | ENNYKTTPPVLDSDGSFFLYSRLTVDKSRWQEGNVFSCSVMHEALHNH |
| | YTQKSLSLSLG (SEQ ID NO: 18) |
| mAb7 full-length light chain | DIVMTQSPDSLAVSLGERATINCKSSQSLWDSGNQKNFLTWYQQKPGQ |
| | PPKLLIYWTSYRESGVPDRFSGSGSGTDFTLTISSLQAEDVAVYYCQN |
| | DYFYPHTFGGGTKVEIKRGTVAAPSVFIFPPSDEQLKSGTASVVCLLN |
| | NFYPREAKVQWKVDNALQSGNSQESVTEQDSKDSTYSLSSTLTLSKAD |
| | YEKHKVYACEVTHQGLSSPVTKSFNRGEC (SEQ ID NO: 19) |
| mAb7 light chain variable | QVQLVQSGAEVKKPGASVKVSCKASGYTFTSYWINWVRQAPGQGLEWM |
| region | GNIYPGSSLTNYNEKFKNRVTMTRDTSTSTVYMELSSLRSEDTAVYYC |
| | ARLSTGTFAYWGQGTLVTVSS (SEQ ID NO: 20) |
| mAB7 and mAB15 heavy | QVQLVQSGAEVKKPGASVKVSCKASGYTFTSYWINWVRQAPGQGLEWM |
| chain variable region | GNIWPGSSLTNYNEKFKNRVTMTRDTSTSTVYMELSSLRSEDTAVYYC |
| | ARLLTGTFAYWGQGTLVTVSS (SEQ ID NO: 21) |
| mAb8 light chain variable | DIVTQSPDSLAVSLGERATINCKSSQSLWDSTNQKNFLTWYQQKPGQP |
| domain with CDRs in bold | PKLLIYWTSTRESGVPDRFSGSGSGTDFTLTISSLQAEDVAVYYCQND |
| (from WO 16/92419) | YFYPLTFGGGTKVEIK (SEQ ID NO: 22) |
| mAb8 heavy chain variable | QVQLVQSGAEVKKPGASVKVSCKASGYTFTSYWINWVRQAPGQGLEWM |
| domain with CDRs in bold | GNIYPGSSLTNYNEKFKNRVTMTRDTSTSTVYMELSSLRSEDTAVYYC |
| (from WO 16/92419) | ARLSTGTFAYWGQTLVTVSS (SEQ ID NO: 23) |
| mAb9 light chain variable | DIVMTQSPDSLAVSLGERATINCKSSQSLWDSGNQKNFLTWYQQKPGQ |
| domain with CDRs in bold | PPKLLIYWTSTRESGVPDRFSGSGSGTDFTLTISSLQAEDVAVYYCQN |
| (from WO 16/92419) | DYFYPLTFGGGTKVEIK (SEQ ID NO: 24) |
| mAb9 heavy chain variable | QVQLVQSGAEVKKPGASVKVSCKASGYTFTSYWINWVRQAPGQGLEWM |
| domain with CDRs in bold | GNIYPGSSITNYNEKFKNRVTMTRDTSTSTVYMELSSLRSEDTAVYYC |
| (from WO 16/92419) | ARLTTGTFAYWGQGTLVTVSS (SEQ ID NO: 25) |
| mAb10 light chain variable | DIVMTQSPDSLAVSLGERATINCKSSQSLWDSGNQKNFLTWYQQKPGQ |
| domain with CDRs in bold | PPKLLIYWTSYRESGVPDRFSGSGSGTDFTLTISSLQAEDVAVYYCQN |
| (from WO 16/92419) | DYFYPLTFGGGTKVEIK (SEQ ID NO: 26) |
| mAb10 heavy chain | QVQLVQSGAEVKKPGASVKVSCKASGYTFTSYWINWVRQAPGQGLEWM |
| variable domain with CDRs | GNIYPGSSITNYNEKFKNRVTMTRDTSTSTVYMELSSLRSEDTAVYYC |
| in bold (from WO 16/92419) | ARLTTGTFAYWGQGTLVTVSS (SEQ ID NO: 27) |
| mAb11 light chain variable | DIVMTQSPDSLAVSLGERATINCKSSQSLWDSGNQKNFLTWYQQKPGQ |
| domain with CDRs in bold | PPKLLIYWTSYRESGVPDRFSGSGSGTDFTLTISSLQAEDVAVYYCQN |
| (from WO 16/92419) | DYFYPHTFGGGTKVEIK (SEQ ID NO: 28) |
| mAb11 heavy chain | QVQLVQSGAEVKKPGASVKVSCKASGYTFTSYWINWVRQAPGQGLEWM |
| variable domain with CDRs | GNIYPGSSITNYNEKFKNRVTMTRDTSTSTVYMELSSLRSEDTAVYYC |
| in bold (from WO 16/92419) | ARLTTGTFAYWGQGTLVTVSS (SEQ ID NO: 29) |
| mAb12 light chain variable | DIVMTQSPDSLAVSLGERATINCKSSQSLWDSTNQKNFLTWYQQKPGQ |
| domain with CDRs in bold | PPKLLIYWTSTRESGVPDRFSGSGSGTDFTLTISSLQAEDVAVYYCQN |
| (from WO 16/92419) | DYFYPLTFGGGTKVEIK (SEQ ID NO: 30) |
| mAb12 heavy chain | QVQLVQSGAEVKKPGASVKVSCKASGYTFTSYWINWVRQAPGQGLEWM |
| variable domain with CDRs | GNIYPGSSITNYNEKFKNRVTMTRDTSTSTVYMELSSLRSEDTAVYYC |
| in bold (from WO 16/92419) | ARLTTGTFAYWGQGTLVTVSS (SEQ ID NO: 31) |
| mAb13 light chain variable | DIVMTQSPDSLAVSLGERATINCKSSQSLWDSGNQKNFLTWYQQKPGQ |
| domain with CDRs in bold | PPKLLIYWTSTRESGVPDRFSGSGSGTDFTLTISSLQAEDVAVYYCQN |
| (from WO 16/92419) | DYFYPLTFGGGTKVEIK (SEQ ID NO: 32) |
| mAb13 heavy chain | QVQLVQSGAEVKKPGASVKVSCKASGYTFTSYWINWVRQAPGQGLEWM |
| variable domain with CDRs | GNIWPGSSLTNYNEKFKNRVTMTRDTSTSTVYMELSSLRSEDTAVYYC |
| in bold (from WO 16/92419) | ARLLTGTFAYWGQGTLVTVSS (SEQ ID NO: 33) |
| mAb14 light chain variable | DIVMTQSPDSLAVSLGERATINCKSSQSLWDSGNQKNFLTWYQQKPGQ |
| domain with CDRs in bold | PPKLLIYWTSYRESGVPDRFSGSGSGTDFTLTISSLQAEDVAVYYCQN |
| (from WO 16/92419) | DYFYPLTFGGGTKVEIK (SEQ ID NO: 34) |
| mAb14 heavy chain | QVQLVQSGAEVKKPGASVKVSCKASGYTFTSYWINWVRQAPGQGLEWM |
| variable domain with CDRs | GNIWPGSSLTNYNEKFKNRVTMTRDTSTSTVYMELSSLRSEDTAVYYC |
| in bold (from WO 16/92419) | ARLLTGTFAYWGQGTLVTVSS (SEQ ID NO: 35) |
| mAb15 light chain variable | DIVMTQSPDSLAVSLGERATINCKSSQSLWDSGNQKNFLTWYQQKPGQ |
| region | PPKLLIYWTSYRESGVPDRFSGSGSGTDFTLTISSLQAEDVAVYYCQN |
| | DYFYPHTFGGGTKVEIK (SEQ ID NO: 36) |
| mAb16 light chain variable | DIVMTQSPDSLAVSLGERATINCKSSQSLWDSTNQKNFLTWYQQKPGQ |
| domain with CDRs in bold | PPKLLIYWTSTRESGVPDRFSGSGSGTDFTLTISSLQAEDVAVYYCQN |
| (from WO 16/92419) | DYFYPLTFGGGTKVEIK (SEQ ID NO: 37) |
| mAb16 heavy chain | QVQLVQSGAEVKKPGASVKVSCKASGYTFTSYWINWVRQAPGQGLEWM |
| variable domain with CDRs | GNIWPGSSLTNYNEKFKNRVTMTRDTSTSTVYMELSSLRSEDTAVYYC |
| in bold (from WO 16/92419) | ARLLTGTFAYWGQGTLVTVSS (SEQ ID NO: 38) |
| AMP224, without signal | LFTVTVPKELYIIEHGSNVTLECNFDTGSHVNLGAITASLQKVENDTS |
| sequence | PHRERATLLEEQLPLGKASFHIPQVQVRDEGQYQCIIIYGVAWDYKYL |
| From WO 2010/027827 | TLKVKASYRKINTHILKVPETDEVELTCQATGYPLAEVSWPNVSVPAN |
| and WO 2011/066342 | TSHSRTPEGLYQVTSVLRLKPPPGRNFSCVFWNTHVRELTLASIDLQS |
| | QMEPRTHPTWEPKSCDKTHTCPPCPAPELLGGPSVFLFPPKPKDTLMI |
| | SRTPEVTCWVDVSHEDPEVKFNWYVDGVEVHNAKTKPREEQYNSTYRW |
| | SVLTVLHQDWLNGKEYKCKVSNKALPAPIEKTISKAKGQPREPQVYTL |
| | PPSRDELTKNQVSLTCLVKGFYPSDIAVEWESNGQPENNYKTTPPVLD |
| | SDGSFFLYSKLTVDKSRWQQGNVFSCSVMHEALHNHYTQKSLSLSPGK |
| | (SEQ ID NO: 39) |

Further examples of an anti-PD-1 antibody include CT-011 (pidilizumab, which is described in WO 09/101611), IBI-308, mDX-400, BGB-108, MEDI-0680, SHR-1210, PF-06801591, PDR-001, GB-226, STI-1110, MEDI-0680 (AMP-514), PDR001, REGN2810, BGB-108, and BGB-A317, or a biosimilar of any of these antibodies.

In some embodiments, a PD-1 inhibitor can be a fusion protein (e.g., an immunoadhesin, e.g., AMP-224, also called B7-DCIg, which is described in WO 10/027827 and WO 11/066342). For example, an immunoadhesin can include an extracellular or PD-1 binding portion of PD-L1 or PD-L2 fused to an antibody constant region (e.g., an Fc region of an immunoglobulin (e.g., a human immunoglobulin) sequence).

In some embodiments, a PD-1 inhibitor can be an aptamer. Non-limiting examples of PD-1 inhibitors that are aptamers are described in, e.g., US 2017/0218369. Additional examples of aptamers that are PD-1 inhibitors are described in Prodeus et al., Mol. Ther. Nucleic Acids 4:e237, 2015; Wang et al., doi: 10.1016/j.biochi.2017.09.006 Biochimie. For example, a PD-1 inhibitor that is an aptamer can include a sequence of one of:

(SEQ ID NO: 40)
GCTACTGTACATCACGCCTCTCCCC,
(SEQ ID NO: 41)
CTACTGTACATCACGCCTCTCCCC,
(SEQ ID NO: 42)
GTACAGTTCCCGTCCCTGCACTACA,
or
(SEQ ID NO: 43)
GTACAGTTCCCGTCCTGCACTACA.

The term “PD-L1 inhibitor” as used herein refers to a molecule that decreases, blocks, inhibits, abrogates or interferes with signal transduction resulting from the interaction of PD-L1 with either one or more of its binding partners, such as PD-1, B7-1. In some embodiments, a PD-L1 inhibitor is a molecule that inhibits the binding of PD-L1 to its binding partners. In a specific aspect, the PD-L1 inhibitor inhibits binding of PD-L1 to PD-1 and/or B7-1. In some embodiments, the PD-L1 inhibitors include anti-PD-L1 antibodies, antigen binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1, B7-1. In one embodiment, a PD-L1 inhibitor reduces the negative co-stimulatory signal mediated by or through cell surface proteins expressed on T lymphocytes mediated signaling through PD-L1 so as render a dysfunctional T-cell less non-dysfunctional.

As used herein, an anti-human PD-L1 antibody refers to an antibody that specifically binds to mature human PD-L1. A mature human PD-L1 molecule consists of amino acids 19-290 of the following sequence:

(SEQ ID NO: 44)
MRIFAVFIFMTYWHLLFAFTVTVPKDLYVVEYGSNMTIECKFPVEKQLD
LAALIVYWEMEDKNIIQFVHGEEDLKVQHSSYRQRARLLKDQLSLGNAA
LQITDVKLQDAGVYRCMISYGGADYKRITVKVNAPYNKINQRILVVDPV
TSEHELTCQAEGYPKAEVIWTSSDHQVLSGKTTTTNSKREEKLFNVTST
LRINTTTNIEFYCTFRRLDPEENHTAELVIPELPLAHPPNERTHLVILG
AILLCLGVALTFIFRLRKGRMMDVKKCGIQDTNSKKQSDTHLEET.

In some embodiments, the PD-L1 inhibitor is an anti-PD-L1 antibody. In some embodiments, the PD-L1 inhibitor is atezolizumab (Tecentriq®) or a biosimilar thereof, or durvalumab (Imfinzi™) or a biosimilar thereof.

A “biosimilar” means an antibody or antigen-binding fragment that has the same primary amino acid sequence as compared to a reference antibody (e.g., nivolumab, pembrolizumab atezolizumab, or durvalumab) and optionally, may have detectable differences in post-translation modifications (e.g., glycosylation and/or phosphorylation) as compared to the reference antibody (e.g., a different glycoform).

In some embodiments, a biosimilar is an antibody or antigen-binding fragment thereof that has a light chain that has the same primary amino acid sequence as compared to a reference antibody (e.g., nivolumab or pembrolizumab) and a heavy chain that has the same primary amino acid sequence as compared to the reference antibody. In some examples, a biosimilar is an antibody or antigen-binding fragment thereof that has a light chain that includes the same light chain variable domain sequence as a reference antibody (e.g., nivolumab or pembrolizumab) and a heavy chain that includes the same heavy chain variable domain sequence as a reference antibody. In some embodiments, a biosimilar can have a similar glycosylation pattern as compared to the reference antibody (e.g., nivolumab or pembrolizumab). In other embodiments, a biosimilar can have a different glycosylation pattern as compared to the reference antibody (e.g., nivolumab or pembrolizumab).

The term “cancer” as used herein refers to or describes the physiological condition in mammals that is typically characterized by unregulated cell growth. In one embodiment of any of the methods disclosed herein, the cancer is a TAM-associated cancer. In one embodiment of any of the methods disclosed herein, the cancer is a c-Met associated cancer.

In one embodiment, the cancer is a TAM-associated cancer. The term “TAM-associated cancer” as used herein refers to cancers associated with or having increased expression and/or activity of one or more of the TAM kinases in a cancer cell or an immune cell (e.g., as compared to a control, e.g., a non-cancerous tissue or cell, or a corresponding tissue or cell from a control subject that does not have cancer). Non-limiting examples of a TAM-associated cancer are described herein. In some embodiments, the TAM-associated cancer is a cancer having a chromosomal translocation that results in the expression of a TMEM87B-MERTK fusion protein (e.g., amino acids 1-55 of TMEM87B and amino acids 433-1000 of MERTK) or a AXL-MBIP fusion protein. A description of an exemplary chromosomal translocation that results in the expression of a TMEM87B-MERTK fusion protein is provided in Shaver et al. (Cancer Res. 76(16):4850-4860, 2016). A description of an exemplary chromosomal translocation that results in the expression of an AXL-MBIP fusion protein is provided in Seo et al. (Genome Res. 22:2109-2119, 2012). Chromosomal translocations or the resulting expression of TMEM87B-MERTK or AXL-MBIP fusion proteins can be detected using In Situ Hybridization (e.g., Fluorescent In Situ Hybridization (FISH)). Chromosomal translocations that result in the expression of TMEM87B-MERTK or AXL-MBIP can be detected by sequencing DNA from a sample obtained from the subject (e.g., blood, plasma, urine, cerebrospinal fluid, saliva, sputum, bronchoalveolar lavage, bile, lymphatic fluid, cyst fluid, stool ascites, or a tumor biopsy obtained from the subject). Exemplary methods that can be used to sequence DNA are known in the art and include, e.g., next-generation sequencing (NGS), traditional PCR, digital PCR, and microarray analysis. Additional methods that can be used to detect chromosomal translocations that result in the expression of TMEM87B-MERTK or AXL-MBIP fusion proteins, or the expression of TMEM87B-MERTK or AXL-MBIP fusion proteins, are known in the art.

Receptor tyrosine kinases (RTKs) are cell surface proteins that transmit signals from the extracellular environment to the cell cytoplasm and nucleus to regulate cellular events such as survival, growth, proliferation, differentiation, adhesion, and migration. All RTKs contain an extracellular ligand binding domain and a cytoplasmic protein tyrosine kinase domain. Ligand binding leads to the dimerization of RTKs, which triggers the activation of the cytoplasmic kinase and initiates downstream signal transduction pathways. RTKs can be classified into distinct subfamilies based on their sequence similarity.

The TAM receptor tyrosine kinases (TYRO3, AXL (also known as UFO) and MER) is an emerging class of innate immune checkpoints that participate in key steps of anti-tumoral immunity (Akalu, T, et al., Immunological Reviews 2017; 276:165-177). TAM kinases are characterized by an extracellular ligand binding domain consisting of two immunoglobulin-like domains and two fibronectin type III domains. Two ligands, growth arrest specific 6 (GAS6) and protein S (ProS), have been identified for TAM kinases. GAS6 can bind to and activate all three TAM kinases, while ProS is a ligand for MER and TYRO3 (Graham et al., 2014, Nature reviews Cancer 14, 769-785).

TAM kinases are ectopically expressed or over-expressed in a wide variety of cancers, including breast, colon, renal, skin, lung, liver, brain, ovarian, prostate, and thyroid malignancies (Graham et al., 2014, Nature Reviews Cancer 14, 769-785; and Linger et al., 2008, Oncogene 32, 3420-3431) and play important roles in tumor initiation and maintenance. When activated, AXL and MER can regulate tumor cell survival, proliferation, migration and invasion, angiogenesis, and tumor-host interactions (Schoumacher, M. et al., Curr. Oncol. Rep. 2017; 19(3); 19). Accordingly, blocking TAM signaling may promote engagement of adaptive immunity and complement T-cell checkpoint blockade (Akalu, T, et al., Immunological Reviews 2017; 276:165-177). Therefore, TAM inhibition represents an attractive approach for targeting another class of oncogenic RTKs (Graham et al., 2014, Nature Reviews Cancer 14, 769-785; and Linger et al., 2008, Oncogene 32, 3420-3431).

AXL was originally identified as a transforming gene from DNA of patients with chronic myelogenous leukemia (O'Bryan et al., 1991, Molecular and Cellular Biology 11, 5016-5031). GAS6 binds to AXL and induces subsequent auto-phosphorylation and activation of AXL tyrosine kinase. AXL activates several downstream signaling pathways including PI3K-AKT, RAF-MAPK, PLC-PKC (Feneyrolles et al., 2014, Molecular Cancer Therapeutics 13, 2141-2148; Linger et al., 2008, Oncogene 32, 3420-3431). Over-expression or overactivation of the AXL protein has been correlated with the promotion of multiple tumorigenic processes. High levels of AXL expression have been associates with poor prognosis in different cancers such as glioblastoma multiforme (Hutterer, M., et al., Clin. Caner Res. 2008, 14, 130-138), breast cancer (Wang, X., Cancer Res. 2013, 73, 6516-6525), lung cancer (Niederst, M. et al, Sci. Signaling, 2013, 6, re6), osteosarcoma (Han, J., Biochem. Biophys. Res. Commun. 2013, 435, 493-500), and acute myeloid leukemia (Ben-Batalla, L., et al., Blood 2013, 122, 2443-2452). AXL is over-expressed or amplified in a variety of malignancies including lung cancer, prostate cancer, colon cancer, breast cancer, melanoma, and renal cell carcinoma (Linger et al., 2008, Oncogene 32, 3420-3431), and over-expression of AXL is correlated with poor prognosis (Linger et al., 2008, Oncogene 32, 3420-3431). AXL activation promotes cancer cell survival, proliferation, angiogenesis, metastasis, and resistance to chemotherapy and targeted therapies. AXL knockdown or AXL antibody can inhibit the migration of breast cancer and NSCLC cancer in vitro, and blocked tumor growth in xenograft tumor models (Li et al., 2009, Oncogene 28, 3442-3455). In pancreatic cancer cells, inhibition of AXL decreased cell proliferation and survival (Koorstra et al., 2009, Cancer Biology & Therapy 8, 618-626). In prostate cancer, AXL inhibition decreased cell migration, invasion, and proliferation (Tai et al., 2008, Oncogene 27, 4044-4055). In triple-negative breast cancer, patients typically present a significant clinical challenge, as they do not respond to the various targeted cancer therapies due to an apparent lack of RTK activation. However, patients with triple-negative breast cancer do show some response to taxane-based chemotherapy and studies have suggested that combining anti-mitotic drugs (e.g., docetaxel) with an AXL inhibitor sensitized cancer cells to the anti-mitotic drug, and AXL in combination with an anti-mitotic drug may be an appropriate combination therapy in this disease setting (Wilson, et al., Cancer Res. 2014, 74(20), 5878-5890).

TAM kinases can contribute to therapeutic resistance by at least three mechanisms: intrinsic survival signaling in tumor cells, induction of TAM kinases as an escape mechanism for tumors that have been treated with oncogene-targeted agents, and immunosuppression in the tumor microenvironment (Graham, et al, Nature Reviews Cancer, 2014, 14, 769-785).

TAM kinases were found to promote resistance to cytotoxic chemotherapies (chemoresistance) in leukemia cells and solid tumor cells (Graham, et al, Nature Reviews Cancer, 2014, 14, 769-785). Transgenic lymphocytes ectopically expressing MER were found to be more resistant to dexamethasone than wild-type lymphocytes (Keating, A. K., et al., Oncogene, 2006, 25, 6092-6100), and stimulation of B-ALL cells with GAS6 increased resistance to cytarabine (Shiozawa, Y., et al., Neoplasia, 2010, 12, 116-127). AXL is induced in acute myeloid leukemia (AML) cells that have been treated with cytotoxic chemotherapies, and it mediates increased chemoresistance (Hong, C. C., et al., Cancer Lett., 2008, 268, 314-324). Chemotherapy-resistant chronic myeloid leukemia (CML) cell lines have upregulated levels of AXL, and shRNA-mediated knockdown of AXL increases chemosensitivity in CML cells and xenograft models (Zhao, Y., et al., Cancer Invest. 2012, 30, 287-294). Similarly, shRNA-mediated MER knock-down sensitizes B-cell acute lymphoblastic leukemia (B-ALL) and T-lineage acute lymphoblastic leukemia (T-ALL) cells to a range of chemotherapies (Linger, R. M., et al., Blood, 2013, 122, 1599-1609; Brandao, L. N., et al., Blood Cancer J., 2013, 3, e101). In solid tumors such as non-small cell lung cancer, pancreatic ductal adenocarcinoma, astrocytoma, lung adenocarcinoma, ovarian cancer, melanoma, and glioblastoma multiforme, overexpression of AXL or MER promotes chemoresistance, and shRNA-mediated inhibition sensitizes cells to treatment with cytotoxic chemotherapies (Linger, R. N., et al., Oncogene, 2013, 32, 3420-3431; Song, X., et al., Cancer, 2011, 117, 734-743; Keating, A. K., et al., Mol. Cancer Ther. 2010, 9, 1298-1307; Lay, J. D., et al., Cancer Res. 2007, 67, 3878-3887; Zhao, Y., et al., Cancer Invest, 2012, 30, 287-294; Macleod, K., Cancer Res. 2005, 65, 6789-6800; Zhu, S., et al., Proc. Natl Acad. Sci. USA, 2009, 106, 17025-17030; Wang, Y., et al., Oncogene 2013, 32, 872-882).

In contrast to chemoresistance, examples of acquired resistance for TAM kinases are currently limited to AXL. AXL is upregulated in imatinib-resistant CML and gastrointestinal stromal tumor (GIST) cell lines and tumor samples (Mahadevan, D., et al., Oncogene, 2007, 26, 3909-3919; Dufies, M., et al., Oncotarget 2011, 2, 874-885; Gioia, R., et al., Blood, 2011, 118, 2211-2221), and siRNA-mediated knockdown of AXL restored imatinib sensitivity to resistant cell lines (Dufies, M., et al.). Similarly, AXL is induced in lapatinib-resistant HER2 (also known as ERBB2)-positive breast cancer cell lines, and AXL inhibition restored lapatinib sensitivity (Liu, L., et al., Cancer Res. 2009, 69, 6871-6878). AXL has been associated with acquired resistance to epidermal growth factor receptor (EGFR) tyrosine kinase inhibitors (e.g., lapatinib and erlotinib) and therapeutic antibodies (e.g., cetuximab) in triple-negative breast cancer (Meyer, A. S. et al., Sci. Signal 2013, 6, ra66), colorectal cancer (Brand, et al., Cancer Res. 2014, 74:5152-5164), head and neck cancer (Kiles, K. M, et al., Mol. Cancer Ther. 2013, 12, 2541-2558) cell lines, and non-small cell lung cancer (Zhang, Nat. Genet. 2013, 44(8), 852-860). AXL has also been associated with acquired resistance to inhibitors targeting other kinases, including PI3Kα inhibitors such as alpelisib (BYL719) in head and neck and esophageal squamous cell carcinomas (Elkabets, et al., Cancer Cell 2015, 27:533-546), MEK inhibitors (e.g., U0126 (1,4-Diamino-2,3-dicyano-1,4-bis(o-aminophenylmercapto)butadiene) and PD 325901 (1,4-Diamino-2,3-dicyano-1,4-bis(o-aminophenylmercapto)butadiene) in triple-negative breast cancer cell lines and melanoma cell lines (Miller, et al., Cancer Discovery 2016, 6:382-39), fibroblast growth factor (FGFR) (Ware, K. E., Oncogenesis 2013, 2, e39), anaplastic lymphoma kinase (ALK) (Kim, H. R., et al., Mol. Oncol. 2013, 7, 1093-1102) and insulin-like growth factor 1 receptor (IGF1R) (Huang, R., Cancer Res. 2010, 70, 7221-7231), and AXL inhibition has been demonstrated to overcome or delay resistance to these inhibitors. AXL is upregulated in NSCLC cell lines and xenografts that are resistant to EGFR tyrosine kinase inhibitors (erlotinib) and antibody drugs (cetuximab) (Brad, T. M., et al., Cancer Res. 2014, 74, 5152-5164; Zhang, Z., et al., Nature Genet. 2012, 44, 852-860), and it is induced in 20% of matched tumor samples taken from patients with NSCLC after development of resistance to the EGFR inhibitor erlotinib.

Regarding MER and AXL dual inhibitors, the normal roles of MER and AXL in preventing or terminating innate immune-mediated inflammation and natural killer (NK) cell responses are subverted in the tumor microenvironment. MER and AXL decrease NK cell antitumor activity, which allows increased metastases.

MER was originally identified as a phospho-protein from a lymphoblastoid expression library (Graham et al., 1995, Oncogene 10, 2349-2359). Both GAS6 and ProS can bind to MER and induce the phosphorylation and activation of MER kinase (Lew et al., 2014. eLife, 3:e03385). Like AXL, MER activation also conveys downstream signaling pathways including PI3K-Akt and Raf-MAPK (Linger et al., 2008, Oncogene 32, 3420-3431). MER is over-expressed in many cancers including multiple myeloma, gastric, prostate, breast, melanoma and rhabdomyosarcoma (Linger et al., 2008, Oncogene 32, 3420-3431). MER knockdown inhibits multiple myeloma cell growth in vitro and in xenograft models (Waizenegger et al., 2014, Leukemia, 1-9). In acute myeloid leukemia, MER knockdown induced apoptosis, decreased colony formation, and increased survival in a mouse model (Lee-Sherick et al., 2013, Oncogene 32, 5359-5368). MER inhibition increased apoptosis, decreased colony formation, increased chemo-sensitivity, and decreased tumor growth in NSCLC (Linger et al., 2013, Oncogene 32, 3420-3431). Similar effects are observed for MER knockdown in melanoma (Schlegel et al., 2013) and glioblastoma (Wang et al., 2013, Oncogene 32, 872-882).

TYRO3 was originally identified through a PCR-based cloning study (Lai and Lemke, 1991, Neuron 6, 691-704). Both ligands, GAS6 and ProS, can bind to and activate Tyro3. TYRO3 also plays a role in cancer growth and proliferation. TYRO3 is over-expressed in melanoma cells, and knockdown of TYRO3 induces apoptosis in these cells (Demarest et al., 2013, Biochemistry 52, 3102-3118).

TAM kinases have emerged as potential immune-oncology targets. The durable clinical responses to immune checkpoint blockade observed in cancer patients clearly indicate that the immune system plays a critical role in tumor initiation and maintenance. Genetic mutations from cancer cells can provide a diverse set of antigens that the immune cells can use to distinguish tumor cells from their normal counterpart. However, cancer cells have evolved multiple mechanisms to evade host immune surveillance. In fact, one hallmark of human cancer is its ability to avoid immune destruction. Cancer cells can induce an immune-suppressive microenvironment by promoting the formation of M2 tumor associated macrophages, myeloid derived suppressor cells (MDSC), and regulatory T cells. Cancer cells can also produce high levels of immune checkpoint proteins such as PD-L1 to induce T cell anergy or exhaustion. It is now clear that tumors co-opt certain immune-checkpoint pathways as a major mechanism of immune resistance (Pardoll, 2012, Cancer 12, 252-264). Antagonizing these negative regulators of T-cell function with antibodies has shown striking efficacy in clinical trials of a number of malignancies including advanced melanoma, non-small cell lung and bladder cancer. While these therapies have shown encouraging results, not all patients mount an anti-tumor response suggesting that other immune-suppressive pathways may also be important.

TAM kinases have been shown to function as checkpoints for immune activation in the tumor milieu. All TAM kinases are expressed in NK cells, and TAM kinases inhibit the antitumor activity of NK cells. LDC1267, a small molecule TAM kinase inhibitor, activates NK cells, and blocks metastasis in tumor models with different histologies (Paolino et al., 2014, Nature 507, 508-512). In addition, MER kinase decreases the activity of tumor associated macrophages through the increased secretion of immune suppressive cytokines such as ILIO and IL4, and decreased production of immune activating cytokines such as IL12 (Cook et al., 2013, The Journal of Clinical Investigation 123, 3231-3242). MER inhibition has been shown to reverse this effect. As a result, MER knockout mice are resistant to PyVmT tumor formation (Cook et al., 2013, Journal of Clinical Investigation 123, 3231-3242). The role of TAM kinases in the immune response is also supported by knockout mouse studies. TAM triple knockout mice (TKO) are viable. However, these mice displayed signs of autoimmune disease including enlarged spleen and lymph nodes, autoantibody production, swollen footpad and joints, skin lesions, and systemic lupus erythematosus (Lu and Lemke, 2001, Science 293, 306-311). This is consistent with the knockout phenotype for approved immune-oncology targets such as CTLA4 and PD-1. Both CTLA-4 and PD-1 knockout mice showed signs of autoimmune disease, and these mice die within a few weeks after birth (Chambers et al., 1997, Immunity 7, 885-895; and Nishimura et al., 2001, Science 291, 319-322). Therefore, inhibition of TAM kinases alone or in combination with other immune therapies may increase the ability of the immune system to make a therapeutically beneficial immune response against the cancer.

In one embodiment of any of the methods disclosed herein, the cancer is a c-Met-associated cancer. The MET receptor tyrosine kinases (e.g., c-Met) controls growth, invasion and metastasis in cancer cells. The c-Met is activated in human cancer by a variety of different molecular mechanisms (see, e.g., Zhang et al., Carcinogenesis 4:345-355, 2016). For example, a c-Met-associated disease or condition (e.g., a c-Met-associated cancer) include: (i) mutations that alter the sequence and increase the activity of c-Met kinase; (ii) mutations in regulatory sequences controlling c-Met expression or regulators of c-Met expression that confer increased expression of c-Met; (iii) mutations that alter the c-Met polypeptide sequence to confer increased c-Met kinase half-life (e.g., a mutation in a MET gene that results in exon 14 skipping during mRNA splicing that results in an increased level of c-Met in a mammalian cell); (iv) methylation of a MET gene (see, e.g., Nones et al., Int. J. Cancer 135:1110-8, 2014); (v) methylation of long interspersed nuclear element (L1) present in the MET intron between exon 2 and exon 3 (Weber et al., Oncogene 29:5775-5784, 2010); (vi) MET gene amplification; or (vii) by simultaneous expression of receptor and ligand, which results in autocrine stimulation of cancer cells (Birchmeier et al., Nat. Rev. Mol. Cell Biol. 4:915-925, 2003).

Exemplary mutations in a MET gene that alter the sequence of a c-Met kinase and increase the activity of c-Met kinase (e.g., as compared to wildtype c-Met kinase) include, but are not limited to those listed in Table D.

TABLE D
Exemplary list of mutations in a MET gene that alter the sequence
of a c-Met kinase and increase the activity of the c-Met kinase

| MET | MET | |
|---|---|---|
| Isoform 1 | Isoform 2 | |
| mutation | mutation | Reference |
| V1092I | V1110I | Schmidt et al., Oncogene 18: 2343-2350, 1999 |
| H1094L | H1112L | Schmidt et al., Oncogene 18: 2343-2350, 1999 |
| H1094R | H1112R | Schmidt et al., Cancer Research |
| | | 58: 1719-1722, 1998 |
| H1094Y | H1112Y | Schmidt et al., Oncogene 18: 2343-2350, 1999 |
| H1106D | H1124D | Schmidt et al., Oncogene 18: 2343-2350, 1999 |
| D1228H | D1246H | Bardelli et al., Proc. Natl. Acad. Sci. |
| | | 95: 14379-14383, 2002 |
| D1228N | D1246N | Bardelli et al., Proc. Natl. Acad. Sci. |
| | | 95: 14379-14383, 2002 |
| Y1230C | Y1248C | Bardelli et al., Proc. Natl. Acad. Sci. |
| | | 95: 14379-14383, 2002 |
| Y1230D | Y1248D | Schmidt et al., Oncogene 18: 2343-2350, 1999 |
| Y1230H | Y1248H | Bardelli et al., Proc. Natl. Acad. Sci. |
| | | 95: 14379-14383, 2002 |
| M1250T | M1268T | Bardelli et al., Proc. Natl. Acad. Sci. |
| | | 95: 14379-14383, 2002 |

Exemplary mutations that alter the c-Met polypeptide sequence to confer increased c-Met kinase half-life (as compared to a wildtype c-Met kinase) include, but are not limited to, the mutations listed in Table E that promote skipping of MET exon 14 during mRNA splicing. Other exemplary mutations that are predicted to promote skipping of MET exon 14 during mRNA splicing include, but are not limited to, those disclosed in Frampton et al., Cancer Discovery 5(8):850-9, 2015; and Heist et al., Oncologist 21(4):481-6, 2016. The portion of the c-Met protein encoded by exon 14, most prominently Y1003 in a DpYR motif, is required for efficient recruitment of the E3 ubiquitin-protein ligase CBL, which targets MET for ubiquitin-mediated degradation (Lee et al., J. Biol. Chem. 269:19457-61, 1994; Lee et al., Exp. Mol. Med. 38:565-73, 2006; Lee et al., Oncogene 33:34-43, 2014). Skipping of MET exon 14 in mRNA splicing results in a c-Met kinase that maintains the reading frame and that demonstrates increased c-Met protein stability and prolonged signaling upon HGF stimulation, leading to increased oncogenic potential (Peschard et al., Mol. Cell 8:995-1004, 2001; Abella et al., Mol. Cell. Biol. 25:9632-45, 2005). Other exemplary mutations that alter the c-Met polypeptide sequence to confer increased c-Met kinase half-life include, but are not limited to, an amino acid substitution at Y1003 (e.g., a Y1003F amino acid substitution) (Peschard et al., Mol. Cell 8:995-1004, 2001).

TABLE E
Exemplary list of mutations that confer skipping of MET exon 14

| | | Altered sequence | |
|---|---|---|---|
| Chromosomal | Reference | (‘—’ denotes | |
| location | sequence | deletion) | Reference |
| chr7: | AAGCTCTTTCTTTCT | | Kong-Beltran et al., Cancer Res. |
| 116411875-116411897 | CTCTGTT | | 66(1): 283-289, 2006 |
| | (SEQ ID NO: 45) | | |
| chr7: | ACCGAGCTACTTTT | | Kong-Beltran et al., Cancer Res. |
| 116412022-116412050 | CCAGAAGGTATATT | | 66(1): 283-289, 2006 |
| | (SEQ ID NO: 46) | | |
| chr7: | G | T | Kong-Beltran et al., Cancer Res. |
| 116412043-116412044 | | | 66(1): 283-289, 2006 |
| chr7: | CCCATGATAGCCGTC | | Onozato et al., J. Thorac. Oncol. |
| 116411854-116411874 | TTTAA | | 4: 5-11, 2009. |
| | (SEQ ID NO: 47) | | |
| chr7: | CTTTCTCTCTG | | Onozato et al., J. Thorac. Oncol. |
| 116411884-116411895 | (SEQ ID NO: 48) | | 4: 5-11, 2009. |
| chr7: | TTCTCTCTGTTTTAA | | Onozato et al., J. Thorac. Oncol. |
| 116411886-116411905 | GATC | | 4: 5-11, 2009. |
| | (SEQ ID NO: 49) | | |
| chr7: | G | A | Onozato et al., J. Thorac. Oncol. |
| 116412043-116412044 | | | 4: 5-11, 2009. |
| chr7: | G | T | Asaoka et al., Biochem. Biophys. |
| 116412043-116412044 | | | Res. Comm. 394: 1042-6,2010. |
| chr7: | CTTTCTCTCTGT | | Jenkins et al., Clin. Lung Cancer |
| 116411884-116411896 | (SEQ ID NO: 50) | | 16: e101-e104, 2015. |
| chr7: | G | C | Waqar et al., J. Thorac. Oncol. |
| 116412042-116412043 | | | 10: e29-31, 2015. |
| chr7: | G | C | Mendenhall et al., J. Thorac. |
| 116412042-116412043 | | | Oncol. 10: e23-34, 2015. |

In some embodiments, the methods described herein are useful for the treatment of tumors and cancers (e.g., TAM-associated cancers and/or c-Met-associated cancers). The TAM-associated cancer and/or c-Met-associated cancer treated can be a primary tumor or a metastatic tumor. In one aspect, the methods described herein are used to treat a solid TAM-associated tumor, for example, melanoma, lung cancer (including lung adenocarcinoma, basal cell carcinoma, squamous cell carcinoma, large cell carcinoma, bronchioloalveolar carcinoma, bronchogenic carcinoma, non-small-cell carcinoma, small cell carcinoma, mesothelioma); breast cancer (including ductal carcinoma, lobular carcinoma, inflammatory breast cancer, clear cell carcinoma, mucinous carcinoma, serosal cavities breast carcinoma); colorectal cancer (colon cancer, rectal cancer, colorectal adenocarcinoma); anal cancer; pancreatic cancer (including pancreatic adenocarcinoma, islet cell carcinoma, neuroendocrine tumors); prostate cancer; prostate adenocarcinoma; urinary tract cancer; ovarian cancer or carcinoma (ovarian epithelial carcinoma or surface epithelial-stromal tumor including serous tumor, endometrioid tumor and mucinous cystadenocarcinoma); liver and bile duct carcinoma (including hepatocellular carcinoma, cholangiocarcinoma, hemangioma); esophageal carcinoma or cancer (including esophageal adenocarcinoma and squamous cell carcinoma); oral and oropharyngeal squamous cell carcinoma; salivary gland adenoid cystic carcinoma: bladder cancer; bladder carcinoma; carcinoma of the uterus (including endometrial cancer or endometrial adenocarcinoma, ocular, uterine papillary serous carcinoma, uterine clear-cell carcinoma, uterine sarcomas and leiomyosarcomas, mixed Mullerian tumors); glioma, glioblastoma, medulloblastoma, and other tumors of the brain; kidney cancers (including renal cancer, renal cell carcinoma, clear cell carcinoma, Wilms' tumor); pituitary adenoma; cancer of the head and neck (including squamous cell carcinomas); cancer of the stomach (gastric cancers, stomach adenocarcinoma, gastrointestinal stromal tumor (GIST)); testicular cancer; germ cell tumor; neuroendocrine tumor; cervical cancer; carcinoids of the gastrointestinal tract, breast, and other organs; signet ring cell carcinoma; mesenchymal tumors including sarcomas (e.g., Kaposi's sarcoma), fibrosarcomas, hemangioma, angiomatosis, hemangiopericytoma, pseudoangiomatous stromal hyperplasia, myofibroblastoma, fibromatosis, inflammatory myofibroblastic tumor, lipoma, angiolipoma, granular cell tumor, neurofibroma, schwannoma, angiosarcoma, liposarcoma, rhabdomyosarcoma, osteosarcoma, leiomyoma, leiomyosarcoma, skin (e.g., squamous cell skin cancer), including melanoma, cervical, retinoblastoma, head and neck cancer, pancreatic, brain, thyroid, testicular, renal, bladder, soft tissue, adrenal gland, urethra, cancers of the penis, myxosarcoma, chondrosarcoma, osteosarcoma, chordoma, malignant fibrous histiocytoma, lymphangiosarcoma, mesothelioma, squamous cell carcinoma; epidermoid carcinoma, malignant skin adnexal tumors, adenocarcinoma, hepatoma, hepatocellular carcinoma, renal cell carcinoma, hypernephroma, cholangiocarcinoma, transitional cell carcinoma, choriocarcinoma, seminoma, embryonal cell carcinoma, glioma anaplastic; glioblastoma multiforme, neuroblastoma, medulloblastoma, malignant meningioma, malignant schwannoma, neurofibrosarcoma, parathyroid carcinoma, medullary carcinoma of thyroid, bronchial carcinoid, pheochromocytoma, Islet cell carcinoma, malignant carcinoid, malignant paraganglioma, melanoma, Merkel cell neoplasm, cystosarcoma phyllodes, salivary cancers, thymic carcinomas, and cancers of the vagina among others.

The methods as described herein may also be useful for treating lymphoma or lymphocytic or myelocytic proliferation disorder or abnormality (e.g., a TAM-associated lymphoma or lymphocytic or myelocytic proliferation disorder or abnormality). For example, the TAM-associated cancer can be a Hodgkin Lymphoma of a Non-Hodgkin Lymphoma. For example, the subject can be suffering from a TAM-associated Non-Hodgkin Lymphoma such as, but not limited to: an AIDS-Related Lymphoma; Anaplastic Large-Cell Lymphoma; Angioimmunoblastic Lymphoma; Blastic N-Cell Lymphoma; Burkitt's Lymphoma: Burkitt-like Lymphoma (Small Non-Cleaved Cell Lymphoma); Chronic Lymphocytic Leukemia/Small Lymphocytic Lymphoma: Cutaneous T-Cell Lymphoma; Diffuse Large B-Cell Lymphoma; Enteropathy-Type T-Cell Lymphoma; Follicular Lymphoma; Hepatosplenic Gamma-Delta T-Cell Lymphoma; Lymphoblastic Lymphoma: Mantle Cell Lymphoma; Marginal Zone Lymphoma; Nasal T-Cell Lymphoma; Pediatric Lymphoma; Peripheral T-Cell Lymphomas; Primary Central Nervous System Lymphoma; T-Cell Leukemias; Transformed Lymphomas; Treatment-Related T-Cell Lymphomas; or Waldenstrom's macroglobulinemia.

The methods as described herein may also be useful to treat a TAM-associated Hodgkin Lymphoma, such as, but not limited to: Nodular Sclerosis Classical Hodgkin's Lymphoma (CHL); Mixed Cellularity CHL; Lymphocyte-depletion CHL; Lymphocyte-rich CHL; Lymphocyte Predominant Hodgkin Lymphoma; or Nodular Lymphocyte Predominant HL.

In one embodiment, the methods as described herein may be useful to treat a patient suffering from a specific TAM-associated T-cell, a B-cell, or a NK-cell based lymphoma, proliferative disorder, or abnormality. For example, the patient can be suffering from a specific TAM-associated T-cell or NK-cell lymphoma, for example, but not limited to: Peripheral T-cell lymphoma, for example, peripheral T-cell lymphoma and peripheral T-cell lymphoma not otherwise specified (PTCL-NOS); anaplastic large cell lymphoma, for example anaplastic lymphoma kinase (ALK) positive. ALK negative anaplastic large cell lymphoma, mantle cell lymphoma, or primary cutaneous anaplastic large cell lymphoma; angioimmunoblastic lymphoma; cutaneous T-cell lymphoma, for example mycosis fungoides, Sezary syndrome, primary cutaneous anaplastic large cell lymphoma, primary cutaneous CD30+ T-cell lymphoproliferative disorder; primary cutaneous aggressive epidermotropic CD8+ cytotoxic T-cell lymphoma; primary cutaneous gamma-delta T-cell lymphoma; primary cutaneous small/medium CD4+ T-cell lymphoma, and lymphomatoid papulosis; Adult T-cell Leukemia/Lymphoma (ATLL); Blastic NK-cell Lymphoma: Enteropathy-type T-cell lymphoma; Hepatosplenic gamma-delta T-cell Lymphoma: Lymphoblastic Lymphoma; Nasal NK/T-cell Lymphomas; Treatment-related T-cell lymphomas; for example lymphomas that appear after solid organ or bone marrow transplantation; T-cell prolymphocyte leukemia; T-cell large granular lymphocytic leukemia; Chronic lymphoproliferative disorder of NK-cells; Aggressive NK cell leukemia; Systemic EBV+ T-cell lymphoproliferative disease of childhood (associated with chronic active EBV infection); Hydroa vacciniforme-like lymphoma; Adult T-cell leukemia/lymphoma; Enteropathy-associated T-cell lymphoma; Hepatosplenic T-cell lymphoma; or Subcutaneous panniculitis-like T-cell lymphoma.

In one embodiment, the methods as described herein may be useful to treat a patient suffering from a specific TAM-associated B-cell lymphoma or proliferative disorder such as, but not limited to: multiple myeloma; Diffuse large B cell lymphoma; Follicular lymphoma; Mucosa-Associated Lymphatic Tissue lymphoma (MALT); Small cell lymphocytic lymphoma; Mantle cell lymphoma (MCL); Burkitt lymphoma; Mediastinal large B cell lymphoma; Waldenstrom's macroglobulinemia; Nodal marginal zone B cell lymphoma (NMZL); Splenic marginal zone lymphoma (SMZL); Intravascular large B-cell lymphoma; Primary effusion lymphoma; or Lymphomatoid granulomatosis; Chronic lymphocytic leukemia/small lymphocytic lymphoma; B-cell prolymphocyte leukemia; Hairy cell leukemia; Splenic lymphoma/leukemia, unclassifiable; Splenic diffuse red pulp small B-cell lymphoma; Hairy cell leukemia-variant; Lymphoplasmacytic lymphoma; Heavy chain diseases, for example, Alpha heavy chain disease, Gamma heavy chain disease, Mu heavy chain disease; Plasma cell myeloma; Solitary plasmacytoma of bone; Extraosseous plasmacytoma; Primary cutaneous follicle center lymphoma; cell/histiocytic rich large B-cell lymphoma; DLBCL associated with chronic inflammation; Epstein-Barr virus (EBV)+DLBCL of the elderly; Primary mediastinal (thymic) large B-cell lymphoma; Primary cutaneous DLBCL, leg type; ALK+large B-cell lymphoma; Plasmablastic lymphoma; Large B-cell lymphoma arising in HHV8-associated multicentric; Castleman disease; B-cell lymphoma, unclassifiable, with features intermediate between diffuse large B-cell lymphoma and Burkitt lymphoma; B-cell lymphoma, unclassifiable, with features intermediate between diffuse large B-cell lymphoma and classical Hodgkin lymphoma; Nodular sclerosis classical Hodgkin lymphoma; Lymphocyte-rich classical Hodgkin lymphoma; Mixed cellularity classical Hodgkin lymphoma; or Lymphocyte-depleted classical Hodgkin lymphoma.

In one embodiment, the methods as described herein may be useful to treat a patient suffering from a TAM-associated leukemia. For example, the subject may be suffering from an acute or chronic TAM-associated leukemia of a lymphocytic or myelogenous origin, such as, but not limited to: Acute lymphoblastic leukemia (ALL); Acute myelogenous leukemia (AML); Chronic lymphocytic leukemia (CLL); Chronic myelogenous leukemia (CML); juvenile myelomonocytic leukemia (JMML); hairy cell leukemia (HCL); acute promyelocyte leukemia (a subtype of AML); T-cell prolymphocyte leukemia (TPLL); large granular lymphocytic leukemia; or Adult T-cell chronic leukemia; large granular lymphocytic leukemia (LGL). In one embodiment, the patient suffers from an acute myelogenous leukemia, for example an undifferentiated AML (MO); myeloblasts leukemia (ML; with/without minimal cell maturation); myeloblastic leukemia (M2; with cell maturation); promyelocytic leukemia (M3 or M3 variant [M3V]); myelomonocytic leukemia (M4 or M4 variant with eosinophilia [M4E]); monocytic leukemia (M5); erythroleukemia (M6); or megakaryoblastic leukemia (M7).

In one embodiment, the compounds and methods described herein are useful for treating a TAM-associated cancer in a patient, wherein the cancer overexpresses AXL, MER, or TYRO3, or a combination thereof, e.g., as compared to a control non-cancerous tissue or a control cell (e.g., from the same or a different subject). In one embodiment, the cancer overexpresses AXL. In one embodiment, the cancer overexpresses MER. In an alternative embodiment, the cancer ectopically expresses MER. In one embodiment, the TAM-associated cancer is breast, colon, renal, skin, lung (including non-small cell lung cancer), liver, gastric, brain (including glioblastoma), ovarian, pancreatic, prostate, glioblastoma multiforme, osteosarcoma, thyroid malignancies, rhabdomyosarcoma, melanoma. acute myeloid leukemia, T-cell acute lymphoid leukemia, B-cell acute lymphoid leukemia, schwannoma, and mantle cell lymphoma.

In one embodiment, the TAM-associated cancer is selected from breast, colon, renal, skin, lung (including non-small cell lung cancer), liver, gastric, brain (including glioblastoma), ovarian, pancreatic, prostate, glioblastoma multiforme, osteosarcoma, thyroid malignancies, rhabdomyosarcoma, and melanoma.

In one embodiment, the TAM-associated cancer is selected from leukemias (including acute myeloid leukemia and chronic myeloid leukemia, B-cell myeloid leukemia (B-CLL), B-cell acute lymphoblastic leukemia, erythroid leukemia, and T-lineage acute lymphoblastic leukemia), non-small cell lung cancer, pancreatic ductal adenocarcinoma, astrocytoma, lung adenocarcinoma, ovarian cancer, melanoma, and glioblastoma multiforme.

In one embodiment, the TAM-associated cancer is selected from chronic myeloid leukemia, gastrointestinal stromal tumors (GIST), breast cancer (e.g., HER2 positive breast cancer and triple negative breast cancer), head and neck cancer, and non-small cell lung cancer.

In some embodiments of any of the methods described herein, the TAM-associated cancer is a cancer having overexpression of a TAM kinase, e.g., as compared to a non-cancerous tissue or cell in the same patient or a different subject. In some embodiments of any of the methods described herein, the TAM-associated cancer is a cancer having ectopic expression of a TAM kinase.

As used herein, the term “ectopic expression” refers to an abnormal gene expression in a cell type, tissue type, or developmental stage in which the gene is not usually expressed.

In some embodiments of any of the methods described herein, the TAM-associated cancer is a cancer having overexpression or ectopic expression of a TYRO3 protein. In some embodiments of any of the methods described herein, the TAM-associated cancer has one or more point mutations in a gene encoding TYRO3 that results in the expression of a TYRO3 that includes one or more amino acid substitutions. In some embodiments of any of the methods described herein, the TAM-associated cancer has a chromosomal translocation which results in the expression of a fusion protein including the kinase domain of TYRO3 and a fusion partner. Non-limiting examples of a TAM-associated cancer having overexpression or ectopic expression of TYRO3, or a mutation in a TYRO3 gene that results in the expression of TYRO3 having one or more point mutations or a TYRO3 fusion protein include: AML, multiple myeloma, lung cancer, melanoma, prostate cancer, endometrial cancer, thyroid cancer, schwannoma, pancreatic cancer, and brain cancer. Non-limiting aspects of TAM-associated cancers having increased expression and/or activity of TYRO3 are listed in Table G. In one embodiment, the methods described herein may be useful for treating a cancer selected from the cancers listed in Table G.

TABLE G
TAM-Associated Cancers Having with Increased
Expression and/or Activity of TYRO3

| | |
|---|---|
| Melanoma | Amino acid substitutions at: |
| | Q67 and/or R462Q, and/or W708fs*5 |
| Lung Cancer | Amino acid substitution at |
| | E340 or N615K in TYRO3 |
| Pancreatic Cancer | Amino acid substitution |
| | R514Q in TYRO3 |
| Colon Cancer | Amino acid substitution G809D |
| | and/or M592I in TYRO3 |
| Brain Cancer | Amino acid substitution |
| | A709T in TYRO3 |
| AML, multiple myeloma, | Overexpression or ectopic |
| lung cancer, melanoma, | expression of TYRO3 |
| prostate cancer, endometrial | |
| cancer, thyroid cancer, | |
| and schwannoma | |

In some embodiments of any of the methods described herein, the TAM-associated cancer is a cancer having overexpression or ectopic expression of an AXL protein. In some embodiments of any of the methods described herein, the TAM-associated cancer has one or more point mutations in a gene encoding AXL that results in the expression of a AXL that includes one or more amino acid substitutions. In some embodiments of any of the methods described herein, the TAM-associated cancer has a chromosomal translocation which results in the expression of a fusion protein including the kinase domain of AXL and a fusion partner. Non-limiting examples of a TAM-associated cancer having overexpression or ectopic expression of AXL, or a mutation in a AXL gene that results in the expression of AXL having one or more point mutations or a AXL fusion protein include: AML, CML, B-CLL, lung cancer, glioblastoma, breast cancer, colorectal cancer, gastric cancer, pancreatic cancer, esophageal cancer, melanoma, squamous cell skin cancer, prostate cancer, endometrial cancer, ovarian cancer, oral squamous cell carcinoma, thyroid cancer, bladder cancer, renal cancer, schwannoma, mesothelioma, Kaposi's sarcoma, osteosarcoma, erythroid leukemia, colon cancer, liver cancer, renal cell carcinoma, osteosarcoma, kidney cancer, PH+CML, non-small cell lung cancer, triple-negative metastatic breast cancer, and HER2+ breast cancer. Non-limiting aspects of TAM-associated cancers having increased expression and/or activity of AXL are listed in Table H. In one embodiment, the methods described herein may be useful for treating a cancer selected from the cancers listed in Table H.

TABLE H
TAM-Associated Cancers Having with Increased Expression and/or Activity of AXL

| | |
|---|---|
| Ovarian Cancer | Amino acid substitutions C24G and/ |
| | or A358V in AXL |
| Melanoma | One or more of the amino acid substitutions |
| | of P36L, R236C, G413W, E431K, A451T, |
| | E535K, G829E, I610V, A666T, S685F, and |
| | R784Q in AXL |
| Colon Cancer | One or more of the amino acid substitutions |
| | of N43T, M580K, and L684P in AXL |
| Skin Cancer | An amino acid substitution of P238L in AXL |
| Gastric Cancer | One or more of the amino acid substitutions |
| | of V289M, R492C, S842F, and P636H in AXL |
| Lung Cancer | One or more of the amino acid substitutions |
| | of R295W, L423Q, K526N, and S599F in AXL |
| Breast Cancer | One or more of the amino acid substitutions |
| | of T343M, E745K, and S747R in AXL |
| Prostate Cancer | An amino acid substitution of R368Q in AXL |
| Pancreatic Cancer | An amino acid substitution of E484D in AXL |
| Kidney Cancer | An amino acid substitution of P742T in AXL |
| AML, CML, B-CLL, lung cancer, | Overexpression or ectopic expression of AXL |
| glioblastoma, breast cancer, colorectal | |
| cancer, gastric cancer, pancreatic cancer, | |
| esophageal cancer, melanoma, squamous | |
| cell skin cancer, prostate cancer, endometrial | |
| cancer, ovarian cancer, oral squamous cell | |
| carcinoma, thyroid cancer, bladder cancer, | |
| renal cancer, schwannoma, mesothelioma, | |
| Kaposi's sarcoma, and osteosarcoma | |

In some embodiments of any of the methods described herein, the TAM-associated cancer is a cancer having overexpression or ectopic expression of a MER protein. In some embodiments of any of the methods described herein, the TAM-associated cancer has one or more point mutations in a gene encoding MER that results in the expression of a MER that includes one or more amino acid substitutions. In some embodiments of any of the methods described herein, the TAM-associated cancer has a chromosomal translocation which results in the expression of a fusion protein including the kinase domain of MER and a fusion partner. Non-limiting examples of a TAM-associated cancer having overexpression or ectopic expression of MER, or a mutation in a MER gene that results in the expression of MER having one or more point mutations or a MER fusion protein include: AML, ALL (B-ALL, T-ALL), lung cancer, glioma, melanoma, prostate cancer, schwannoma, mantle cell lymphoma, rhabdomyosarcoma, pancreatic cancer, breast cancer, gastric cancer, pituitary adenoma, urinary tract cancer, kidney cancer, liver cancer, colon cancer, and breast cancer. Non-limiting aspects of MER-associated cancers having increased expression and/or activity of MER are listed in Table I. In one embodiment, the methods described herein may be useful for treating a cancer selected from the cancers listed in Table I.

TABLE I
TAM-Associated Cancers Having with Increased Expression and/or Activity of MER

| | |
|---|---|
| Melanoma | One or more amino acid substitutions of P40S, |
| | V861I, K923R, and P802S in MER |
| Lung Cancer | One or more amino acid substitutions of |
| | S159F, I431F, S905F, P672S, N718Y, and |
| | M790V in MER |
| Urinary Tract Cancer | One or more amino acid substitutions of |
| | E204K, L586F, and S626C in MER |
| Gastric Cancer | An amino acid substitution of S428G in MER |
| Kidney Cancer | Amino acid substitutions of A446G and/or |
| | P958L in MER |
| Liver Cancer | One or more amino acid substitutions of |
| | N454S, V873I, and D983N in MER |
| Lymphoma | An amino acid substitution of W485S/C in MER |
| Colon Cancer | One or more amino acid substitutions of |
| | D990N, L688M, and R722 in MER |
| Breast Cancer | An amino acid substitution of G594R in MER |
| Head and Neck Cancer | An amino acid substitution of A708S in MER |
| AML, ALL, lung cancer, glioma, melanoma, | Overexpression or ectopic expression of MER |
| prostate cancer, schwannoma, mantle cell | |
| lymphoma, and rhabdomyosarcoma | |

In one embodiment of any of the methods disclosed herein for treating cancer, the cancer is a TAM-associated cancer.

In one embodiment of any of the methods disclosed herein for treating cancer, the cancer is a TAM-associated cancer having overexpression of a TAM kinase.

In one embodiment of any of the methods disclosed herein for treating cancer, the cancer is a TAM-associated cancer having ectopic expression of a TAM kinase.

In one embodiment of any of the methods disclosed herein for treating cancer, the cancer is a TAM-associated cancer associated with or having abnormal (e.g., increased) expression, level, and/or activity of one or more of the TAM kinases, e.g., as compared to a non-cancerous tissue or cell from the patient or a different subject.

In one embodiment, the TAM-associated cancer is selected from the group consisting of: gastrointestinal stromal tumor (GIST), acute myeloid leukemia (AML), acute lymphocytic leukemia (ALL), chronic myeloid leukemia (CML), B-cell chronic myeloid leukemia (B-CLL), lung cancer, glioblastoma, breast cancer, colorectal cancer, gastric cancer, glioma, pancreatic cancer, esophageal cancer, mantle cell lymphoma, melanoma, squamous cell skin cancer, prostate cancer, endometrial cancer, ovarian cancer, oral squamous cell carcinoma, thyroid cancer, bladder cancer, renal cancer, schwannoma, mesothelioma, Kaposi's sarcoma, osteosarcoma, rhabdomyosarcoma, erythroid leukemia, colon cancer, liver cancer, renal cell carcinoma, pituitary adenoma, urinary tract cancer, kidney cancer, head and neck cancer, brain cancer, and non-small cell lung cancer.

In one embodiment, the TAM-associated cancer is selected from the group consisting of: acute myeloid leukemia (AML), multiple myeloma, lung cancer, melanoma, prostate cancer, endometrial cancer, thyroid cancer, schwannoma, pancreatic cancer, and brain cancer.

In one embodiment, the TAM-associated cancer is cervical cancer, gastric cancer, esophageal cancer, hepatocellular carcinoma, melanoma (e.g., mucosal or cutaneous), Merkel Cell Carcinoma, microsatellite instability-high (MSI-H) tumors, non-small cell lung cancer, head and neck squamous cell carcinoma, small cell lung cancer, renal cell carcinoma, or urothelial carcinoma.

In one embodiment, the TAM-associated cancer is a cancer having overexpression or ectopic expression of an AXL protein. In one embodiment, the TAM-associated cancer is selected from the group consisting of: gastrointestinal stromal tumor (GIST), acute myeloid leukemia (AML), acute lymphocytic leukemia (ALL), chronic myeloid leukemia (CML), B-cell chronic myeloid leukemia (B-CLL), lung cancer, glioblastoma, breast cancer, colorectal cancer, gastric cancer, pancreatic cancer, prostate cancer, esophageal cancer, melanoma, squamous cell skin cancer, endometrial cancer, ovarian cancer, oral squamous cell carcinoma, thyroid cancer, bladder cancer, renal cancer, schwannoma, mesothelioma, osteosarcoma, erythroid leukemia, colon cancer, liver cancer, renal cell carcinoma, kidney cancer, non-small cell lung cancer, and triple-negative metastatic breast cancer.

In one embodiment of any of the methods disclosed herein for treating cancer, the cancer is a c-Met associated cancer. In one embodiment c-Met-associated cancers include, but are not limited to those listed in Table F. In one embodiment c-Met-associated cancers is selected from a cancer listed in Table F.

TABLE F
Exemplary c-Met-associated cancers exhibiting
increased expression and/or activity of c-Met

| Cancer type | Type of genetic alterations | Reference |
|---|---|---|
| Gastrointestinal | MET gene amplification; | Mo et al., Chronic Dis. |
| cancer (GI); | Amino acid substitution in | Transl. Med. 3(3): 148- |
| Gastric cancer | kinase domain (e.g., an amino | 153, 2017; Tovar et al., |
| | acid substitution at position | Ann. Transl. Med. |
| | 1108, e.g., an A1108S amino | 5(10): 205, 2017; Asaoka |
| | acid substitution); | et al., Biochem. Biophys. |
| | point mutation conferring | Res. Comm. 394: 1042- |
| | skipping of MET exon 14 | 6, 2010. |
| | during mRNA splicing (e.g., | |
| | chr7: 116412043-116412044, | |
| | G to T mutation) | |
| Colorectal | Amino acid substitution at | Zenali et al., |
| Adenocarcinoma | position 375 (e.g., a N375S | Oncoscience 2(5): 533- |
| | amino acid substitution); an | 541, 2015. |
| | amino acid substitution at | |
| | position 1010 (e.g., a T1010I | |
| | amino acid substitution); an | |
| | amino acid substitution at | |
| | position 988 (e.g., a R988C | |
| | amino acid substitution); an | |
| | amino acid substitution at | |
| | position 1253 (e.g., a Y1253D | |
| | amino acid substitution); and | |
| | an amino acid substitution at | |
| | position 1248 (e.g., a Y1248H | |
| | amino acid substitution) | |
| Colorectal | MET gene amplification; | Zeng et al., Cancer Lett. |
| carcinoma (CRC) | MET overexpression; | 265: 258-269, 2008; |
| | amino acid substitutions in JM | Kong-Beltran et al., |
| | domain of c-Met kinase (e.g., | Cancer Res. 66: 283-9, |
| | an amino acid substitution at | 2006; Tovar et al., Ann. |
| | position 970 (e.g., an R970C | Transl. Med. 5(10): 205, |
| | amino acid substitution) and an | 2017. |
| | amino acid substitution at | |
| | position 992 (e.g., a T992I | |
| | amino acid substitution) | |
| Non-small cell | Point mutation conferring | Ichimura et al., Jpn J. |
| lung cancer | skipping of MET exon 14 | Cancer Res. 87: 1063- |
| (NSCLC) | during mRNA splicing; | 1069, 1996; Ma et al., |
| | MET gene amplification; | Cancer Res. 63: 6272- |
| | amino acid substitutions in c- | 81, 2003; Kong-Beltran |
| | Met kinase domain (e.g., an | et al., Cancer Res. |
| | amino acid substitution at | 66: 283-9, 2006; Tovar et |
| | position 970 (e.g., a R970C | al., 2017, Ann. Transl. |
| | amino acid substitution), an | Med. 5(10): 205, 2017 |
| | amino acid substitution at | |
| | position 988 (e.g., a R988C | |
| | amino acid substitution); an | |
| | amino acid substitution at | |
| | position 1010 (e.g., a T1010I | |
| | amino acid substitution); an | |
| | amino acid substitution at | |
| | position 1058 (e.g., a S1058P | |
| | amino acid substitution)); | |
| | amino acid substitution in the | |
| | JM domain of c-Met kinase | |
| | (e.g., an amino acid | |
| | substitution at position 988 | |
| | (e.g., a R988C amino acid | |
| | substitution), an amino acid | |
| | substitution at position 1010 | |
| | (e.g., a T1010I amino acid | |
| | substitution), an amino acid | |
| | substitution at position 1058 | |
| | (e.g., a S1058P amino acid | |
| | substitution), an amino acid | |
| | substitution at position 970 | |
| | (e.g., a R970C amino acid | |
| | substitution), and an amino | |
| | acid substitution at position | |
| | 992 (e.g., a T992I amino acid | |
| | substitution)). | |
| Heptacellular | MET overexpression; | Goyal et al., Clin. |
| carcinoma (HCC) | Amino acid substitutions in | Cancer Res. 19: 2310- |
| | kinase domain of c-Met (e.g., | 2318, 2013; Tovar et al., |
| | an amino acid substitution at | Ann. Transl. Med. |
| | position 1191 (e.g., a T1191I | 5(10): 205, 2017; Zenali |
| | amino acid substitution), an | et al., Oncoscience |
| | amino acid substitution at | 2(5): 533-541, 2015 |
| | position 1262 (e.g., a J1262R | |
| | amino acid substitution), or an | |
| | amino acid substitution at | |
| | position 1268 (e.g., a M1268T | |
| | or an M1268I amino acid | |
| | substitution)); an amino acid | |
| | substitution at position 375 | |
| | (e.g., an N375S amino acid | |
| | substitution); an amino acid | |
| | substitution at position 988 | |
| | (e.g., a R988C amino acid | |
| | substitution) | |
| Hereditary | Amino acid substitutions in the | Tovar et al., Ann. Transl. |
| papillary renal | kinase domain of c-Met (e.g., | Med. 5(10): 205, 2017 |
| carcinoma (HPRC) | an amino acid substitution at | |
| | position 112 (e.g., a H112R, a | |
| | H112L, or a H112I amino acid | |
| | substitution), an amino acid | |
| | position as position 1230 (e.g., | |
| | a Y1230C, a Y1230H, ora | |
| | Y1230D amino acid | |
| | substitution), an amino acid | |
| | substitution at position 1246 | |
| | (e.g., a D1246N amino acid | |
| | substitution), an amino acid | |
| | substitution at position 1248 | |
| | (e.g., a Y1248C amino acid | |
| | substitution), an amino acid | |
| | substitution at position 1268 | |
| | (e.g., a M1268T amino acid | |
| | substitution or a M1268I amino | |
| | acid substitution). | |
| Papillary | Amino acid substitutions in the | Jeffers et al., Proc. Natl. |
| renal carcinoma | kinase domain of c-Met | Acad. Sci. U.S.A. |
| | | 94(21): 11445-11450, |
| | | 1997; Schmidt et al., Nat. |
| | | Genet. 16: 68-73, 1997; |
| | | Schmidt et al., Oncogene |
| | | 18: 2343-50, 1991. |
| Melanoma | An amino acid substitution at | Zenali et al., |
| | position 375 (e.g., a N375S | Oncoscience 2(5): 533- |
| | amino acid substitution); an | 541, 2015. |
| | amino acid substitution at | |
| | position 988 (e.g., a R988C | |
| | amino acid substitution); an | |
| | amino acid substitution at | |
| | position 1010 (e.g., a T1010I | |
| | amino acid substitution). | |
| Gastric | An amino acid substitution at | Zenali et al., |
| adenocarcinoma | position 375 (e.g., an N375S | Oncoscience 2(5): 533- |
| | amino acid substitution). | 541, 2015. |
| Appendiceal | An amino acid substitution at | Zenali et al., |
| adenocarcinoma | position 375 (e.g., an N375S | Oncoscience 2(5): 533- |
| | amino acid substitution); an | 541, 2015. |
| | amino acid substitution at | |
| | position 988 (e.g., a R988C | |
| | amino acid substitution) | |
| Duodenal | An amino acid substitution at | Zenali et al., |
| adenocarcinoma | position 375 (e.g., an N375S | Oncoscience 2(5): 533- |
| | amino acid substitution) | 541, 2015. |
| Pancreatic | An amino acid substitution at | Zenali et al., |
| adenocarcinoma | position 375 (e.g., an N375S | Oncoscience 2(5): 533- |
| | amino acid substitution) | 541, 2015. |
| Lung | An amino acid substitution at | Zenali et al., |
| adenocarcinoma | position 375 (e.g., an N375S | Oncoscience 2(5): 533- |
| | amino acid substitution); an | 541, 2015. |
| | amino acid substitution at | |
| | position 988 (e.g., a R988C | |
| | amino acid substitution); an | |
| | amino acid substitution at | |
| | position 1010 (e.g., a T1010I | |
| | amino acid substitution) | |
| Thyroid | An amino acid substitution at | Zenali et al., |
| papillary | position 375 (e.g., an N375S | Oncoscience 2(5): 533- |
| carcinoma | amino acid substitution) | 541, 2015. |
| Thyroid | An amino acid substitution at | Zenali et al., |
| medullary | position 1010 (e.g., a T1010I | Oncoscience 2(5): 533- |
| carcinoma | amino acid substitution) | 541, 2015. |
| Ewing | An amino acid substitution at | Zenali et al., |
| sarcoma | position 1010 (e.g., a T1010I | Oncoscience 2(5): 533- |
| | amino acid substitution) | 541, 2015. |
| Prostate | An amino acid substitution at | Zenali et al., |
| adenocarcinoma | position 375 (e.g., an N375S | Oncoscience 2(5): 533- |
| | amino acid substitution) | 541, 2015. |
| Squamous cell | An amino acid substitution at | Zenali et al., |
| carcinoma of | position 375 (e.g., an N375S | Oncoscience 2(5): 533- |
| the head and | amino acid substitution); an | 541, 2015. |
| neck and cervix | amino acid substitution at | |
| | position 988 (e.g., a R988C | |
| | amino acid substitution); an | |
| | amino acid substitution at | |
| | position 1010 (e.g., an T1010I | |
| | amino acid substitution) | |
| Renal cell | An amino acid substitution at | Zenali et al., |
| carcinoma | position 375 (e.g., an N375S | Oncoscience 2(5): 533- |
| | amino acid substitution); an | 541, 2015; Schmidt et |
| | amino acid substitution at | al., Oncogene 18: 2343- |
| | position 1092 (e.g., a V1092I | 2350, 1999; Schmidt et |
| | amino acid substitution); an | al., Cancer Research |
| | amino acid substitution at | 58: 1719-1722, 1998; |
| | position 1094 (e.g., a H1094L, | Bardelli et al., Proc. Natl. |
| | a H1094R, or a H1094Y amino | Acad. Sci. 95: 14379- |
| | acid substitution); an amino | 14383, 2002. |
| | acid substitution at position | |
| | 1106 (e.g., a H1106D amino | |
| | acid substitution); an amino | |
| | acid substitution at position | |
| | 1228 (e.g., a D1228H ora | |
| | D1228N amino acid | |
| | substitution); an amino acid | |
| | substitution at position 1230 | |
| | (e.g., a Y1230C, a Y1230D, or | |
| | a Y1230H amino acid | |
| | substitution); an amino acid | |
| | substitution at position 1250 | |
| | (e.g., a M1250T amino acid | |
| | substitution) | |
| Pheochromocytoma | An amino acid substitution at | Zenali et al., |
| and composite | position 375 (e.g., an N375S | Oncoscience 2(5): 533- |
| pheochromocytoma | amino acid substitution); an | 541, 2015. |
| | amino acid substitution at | |
| | position 988 (e.g., an R988C | |
| | amino acid substitution) | |
| Ovarian | An amino acid substitution at | Zenali et al., |
| serous | position 375 (e.g., an N375S | Oncoscience 2(5): 533- |
| carcinoma | amino acid substitution); an | 541, 2015. |
| | amino acid substitution at | |
| | position 1010 (e.g., a 1010I | |
| | amino acid substitution) | |
| Ovarian clear | An amino acid substitution at | Zenali et al., |
| cell carcinoma | position 375 (e.g., an N375S | Oncoscience 2(5): 533- |
| | amino acid substitution) | 541, 2015. |
| Ovarian mixed | An amino acid substitution at | Zenali et al., |
| carcinoma | position 1010 (e.g., a T1010I | Oncoscience 2(5): 533- |
| | amino acid substitution) | 541, 2015. |
| Peritoneal | An amino acid substitution at | Zenali et al., |
| serous carcinoma | position 1010 (e.g., a T1010I | Oncoscience 2(5): 533- |
| | amino acid substitution) | 541, 2015. |
| Breast ductal | An amino acid substitution at | Zenali et al., |
| adenocarcinoma | position 375 (e.g., an N375S | Oncoscience 2(5): 533- |
| | amino acid substitution); an | 541, 2015. |
| | amino acid substitution at | |
| | position 1010 (e.g., a T1010I | |
| | amino acid substitution). | |
| Uterine | An amino acid substitution at | Zenali et al., |
| leiomyosarcoma | position 375 (e.g., an N375S | Oncoscience 2(5): 533- |
| | amino acid substitution); amino | 541, 2015. |
| | acid substitution at position | |
| | 1010 (e.g., a T1010I amino | |
| | acid substitution) | |
| Uterine | An amino acid substitution at | Zenali et al., |
| endometrioid | position 375 (e.g., an N375S | Oncoscience 2(5): 533- |
| adenocarcinoma | amino acid substitution); an | 541, 2015. |
| | amino acid substitution at | |
| | position 1010 (e.g., an T1010I | |
| | amino acid substitution). | |
| Uterine malignant | An amino acid substitution at | Zenali et al., |
| mixed Mullerian | position 375 (e.g., an N375S | Oncoscience 2(5): 533- |
| tumor | amino acid substitution) | 541, 2015. |
| Glioblastoma | An amino acid substitution at | Zenali et al., |
| | position 375 (e.g., an N375S | Oncoscience 2(5): 533- |
| | amino acid substitution) | 541, 2015. |
| Anaplastic | An amino acid substitution at | Zenali et al., |
| glioma | position 375 (e.g., an N375S | Oncoscience 2(5): 533- |
| | amino acid substitution) | 541, 2015. |
| Oligodendroglioma | An amino acid substitution at | Zenali et al., |
| | position 1010 (e.g., an T1010I | Oncoscience 2(5): 533- |
| | amino acid substitution) | 541, 2015. |
| Desmoplastic | An amino acid substitution at | Zenali et al., |
| small round | position 375 (e.g., an N375S | Oncoscience 2(5): 533- |
| cell tumor | amino acid substitution) | 541, 2015. |
| Squamous cell | An amino acid substitution at | Zenali et al., |
| carcinoma of | position 375 (e.g., N375S | Oncoscience 2(5): 533- |
| rectum | amino acid substitution) | 541, 2015. |
| Salivary gland | An amino acid substitution at | Zenali et al., |
| carcinoma | position 375 (e.g., an N375S | Oncoscience 2(5): 533- |
| | amino acid substitution) | 541, 2015. |
| Heart | An amino acid substitution at | Zenali et al., |
| angiosarcoma | position 375 (e.g., a N375S | Oncoscience 2(5): 533- |
| | amino acid substitution); an | 541, 2015. |
| | amino acid substitution at | |
| | position 1010 (e.g., a T1010I | |
| | amino acid substitution) | |
| Gastrointestinal | An amino acid substitution at | Zenali et al., |
| stromal tumor | position 1010 (e.g., a T1010I | Oncoscience 2(5): 533- |
| (GIST) | amino acid substitution); an | 541, 2015. |
| | amino acid substitution at | |
| | position 988 (e.g., an R988C | |
| | amino acid substitution) | |
| Invasive | An amino acid substitution at | Zenali et al., |
| thymoma | position 375 (e.g., an N375S | Oncoscience 2(5): 533- |
| | amino acid substitution) | 541, 2015. |
| Spindle | An amino acid substitution at | Zenali et al., |
| sarcoma | position 375 (e.g., an N375S | Oncoscience 2(5): 533- |
| | amino acid substitution) | 541, 2015. |

In some embodiments, the methods described herein are useful for treating a c-Met associated cancer expressing a c-Met kinase that is resistant (e.g., to at least some extent as compared to a wildtype c-Met kinase) to a c-Met inhibitor (e.g., a Type I c-Met inhibitor). Non-limiting examples of amino acid substitutions that result in resistance of c-Met to a c-Met inhibitor (e.g., a Type I c-Met inhibitor) include: an amino acid substitution at position 1092 (e.g., a V1092I amino acid substitution in isoform 1 of c-Met or a V1110I amino acid substitution in isoform 2 of c-Met); an amino acid substitution at position 1094 (e.g., a H1094L amino acid substitution in isoform 1 of c-Met or a H1112L amino acid substitution in isoform 2 of c-Met; an H1094Y amino acid substitution in isoform 1 of c-Met or an H1112Y amino acid substitution in isoform 2 of c-Met); an amino acid substitution at position 1155 (e.g., a V1155L amino acid substitution in isoform 1 or a V1173L amino acid substitution in isoform 2 of c-Met); an amino acid substitution at position 1163 (e.g., a G1163R amino acid substitution in isoform 1 of c-Met or a G1181R amino acid substitution in isoform 2 of c-Met); an amino acid substitution at position 1195 (e.g., an L1195F amino acid substitution in isoform 1 of c-Met or a L1213F amino acid substitution in isoform 2 of c-Met; an L1195V amino acid substitution in isoform 1 of c-Met or an L1213V amino acid substitution in isoform 2 of c-Met); an amino acid substitution at position 1200 (e.g., an F1200I amino acid substitution in isoform 1 of c-Met or an F1218I amino acid substitution in isoform 2 of c-Met); an amino acid substitution at position 1211 (e.g., an M1211L amino acid substitution in isoform 1 of c-Met or an M1229L amino acid substitution in isoform 2 of c-Met); an amino acid substitution at position 1228 (e.g., a D1228A amino acid substitution in isoform 1 of c-Met or a D1246A amino acid substitution in isoform 2 of c-Met; a D1228G amino acid substitution in isoform 1 of c-Met or a D1246G amino acid substitution in isoform 2 of c-Met; a D1228H amino acid substitution in isoform 1 of c-Met or a D1246H amino acid substitution in isoform 2 of c-Met; a D1228N amino acid substitution in isoform 1 of c-Met or a D1246N amino acid substitution in isoform 2 of c-Met; a D1228V amino acid substitution in isoform 1 of c-Met or a D1246V amino acid substitution in isoform 2 of c-Met; or a D1228Y amino acid substitution in isoform 1 of c-Met or a D1246Y amino acid substitution in isoform 2 of c-Met); an amino acid substitution at position 1230 (e.g., a Y1230C amino acid substitution in isoform 1 of c-Met or a Y1248C amino acid substitution in isoform 2 of c-Met; a Y1230H amino acid substitution in isoform 1 of c-Met or a Y1248H amino acid substitution in isoform 2 of c-Met; or a Y1230S amino acid substitution in isoform 1 of c-Met or a Y1248S amino acid substitution in isoform 2 of c-Met); or an amino acid substitution at position 1250 (e.g., a M1250T amino acid substitution in isoform 1 of c-Met or a M1268T amino acid substitution in isoform 2 of c-Met). Non-limiting examples of Type I inhibitors include crizotinib (PF-02341066), capmatinib, NVP-BVU972, AMG 337, bozitinib, glumetinib, savolitinib, and tepotinib. In some embodiments, amino acid substitutions that result in resistance of c-Met to a Type 1 c-Met inhibitor include L1195V, F1200I, D1228H, D1228N, Y1230C, Y1230H, and Y1230S.

In some embodiments, the methods described herein are useful for treating a c-Met associated cancer having a chromosomal translocation that result in a fusion protein including the c-Met kinase domain, where the fusion protein has increased c-Met activity as compared to a wildtype c-Met kinase (e.g., a Met-TPR fusion protein (Rodrigues et al., Mol. Cell. Biol. 13:6711-6722, 1993) and the fusion protein/chromosomal translocation described in Cooper et al., Nature 311(5981):29-33, 1984.

In one embodiment, the c-Met associated cancer is selected from the group of gastrointestinal cancer (GI), gastric cancer, colorectal adenocarcinoma, colorectal carcinoma (CRC), non-small cell lung cancer (NSCLC), hepatocellular carcinoma (HCC), hereditary papillary renal carcinoma (HPRC), papillary renal carcinoma, melanoma, gastric adenocarcinoma, appendiceal adenocarcinoma, duodenal adenocarcinoma, pancreatic adenocarcinoma, lung adenocarcinoma, thyroid papillary carcinoma, thyroid medullary carcinoma, Ewing sarcoma, prostate adenocarcinoma, squamous cell carcinoma of the head and neck and cervix, renal cell carcinoma, pheochromocytoma and composite pheochromocytoma, ovarian serous carcinoma, ovarian clear cell carcinoma, ovarian mixed carcinoma, peritoneal serous carcinoma, breast ductal adenocarcinoma, uterine leiomyosarcoma, uterine endometrioid adenocarcinoma, uterine malignant mixed Mullerian tumor, glioblastoma, anaplastic glioma, oligodendroglioma, desmoplastic small round cell tumor, squamous cell carcinoma of rectum, salivary gland carcinoma, heart angiosarcoma, gastrointestinal stromal tumor, invasive thymoma, and spindle sarcoma.

In one embodiment, the cancer is an advanced or metastatic solid tumor.

In one embodiment, the cancer is a cancer that is responsive to immunotherapy.

In one embodiment, the cancer is a cancer that is resistant to standard therapy.

In one embodiment, the cancer is a cancer for which no standard therapy is available.

In one embodiment, the cancer is a measurable lesion or a non-measurable lesion that has not been previously irradiated, as defined by RECIST version 1.1

In some embodiments of any of the methods described herein, the subject is identified or diagnosed as having a TAM-associated cancer (e.g., any of the TAM-associated cancers described herein, e.g., having any of the exemplary TAM mutations described herein).

In some embodiments of any of the methods described herein, the subject is identified or diagnosed as having a c-Met-associated cancer (e.g., any of the c-Met-associated cancers described herein, e.g., having any of the exemplary c-Met mutations described herein).

The term “combination therapy” as used herein refers to a dosing schedule of two different therapeutically active agents i.e., Compound 1 or a pharmaceutically acceptable salt thereof and a PD-1 inhibitor or a PD-L1 inhibitor during a period of time, wherein the therapeutically active agents are administered together or separately in a manner prescribed by a medical care taker or according to a regulatory agency as defined herein. In one embodiment, a combination therapy comprises a combination of Compound 1 or a pharmaceutically acceptable salt thereof, and a PD-1 inhibitor. In one embodiment, a combination therapy comprises a combination of Compound 1 or a pharmaceutically acceptable salt thereof, and a PD-1 inhibitor which is nivolumab or a biosimilar thereof. In one embodiment, a combination therapy comprises a combination of Compound 1 or a pharmaceutically acceptable salt thereof, and a PD-1 inhibitor which is pembrolizumab or a biosimilar thereof. In one embodiment, a combination therapy comprises a combination of Compound 1 or a pharmaceutically acceptable salt thereof, and a PD-L1 inhibitor. In one embodiment, a combination therapy comprises a combination of Compound 1 or a pharmaceutically acceptable salt thereof, and a PD-1 inhibitor which is sasanlimab or a biosimilar thereof.

A combination therapy according to any of the methods disclosed herein can be administered to a patient for a period of time. In some embodiments, the period of time occurs following the administration of a different cancer therapeutic treatment/agent or a different combination of cancer therapeutic treatments/agents to the patient. In some embodiments, the period of time occurs before the administration of a different cancer therapeutic treatment/agent or a different combination of cancer therapeutic treatments/agents to the patient. In some embodiments, administration of the PD-1 inhibitor and administration of Compound 1 or a pharmaceutically acceptable salt thereof occurs at substantially the same time. In some embodiments, administration of the PD-1 inhibitor to the patient occurs prior to administration of Compound 1 or a pharmaceutically acceptable salt thereof to the patient, during the dosing period. In some embodiments, administration of Compound 1 or a pharmaceutically acceptable salt thereof to the patient occurs prior to administration of the PD-1 inhibitor to the patient, during the dosing period.

As used herein, an “effective dosage” or “effective amount” or “therapeutically effective amount” of a drug, compound, or pharmaceutical composition is an amount sufficient to effect any one or more beneficial or desired results. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as reducing incidence or amelioration of one or more symptoms of various diseases or conditions (such as for example cancer), decreasing the dose of other medications required to treat the disease, enhancing the effect of another medication, and/or delaying the progression of the disease. An effective dosage can be administered in one or more administrations. For purposes of this invention, an effective dosage of a drug, compound, or pharmaceutical composition is an amount sufficient to accomplish prophylactic or therapeutic treatment either directly or indirectly. As is understood in the clinical context, an effective dosage of a drug, compound, or pharmaceutical composition may be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved. In reference to the treatment of cancer, an effective amount may also refer to that amount which has the effect of (1) reducing the size of the tumor, (2) inhibiting (that is, slowing to some extent, preferably stopping) tumor metastasis emergence, (3) inhibiting to some extent (that is, slowing to some extent, preferably stopping) tumor growth or tumor invasiveness, and/or (4) relieving to some extent (or, preferably, eliminating) one or more signs or symptoms associated with the cancer. Therapeutic or pharmacological effectiveness of the doses and administration regimens may also be characterized as the ability to induce, enhance, maintain or prolong disease control and/or overall survival in patients with these specific tumors, which may be measured as prolongation of the time before disease progression

“Tumor” as it applies to a subject diagnosed with, or suspected of having, a cancer refers to a malignant or potentially malignant neoplasm or tissue mass of any size and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

The term “advanced”, as used herein, as it relates to solid tumors, includes locally advanced (non-metastatic) disease and metastatic disease. Locally advanced solid tumors, which may or may not be treated with curative intent, and metastatic disease, which cannot be treated with curative intent are included within the scope of “advanced solid tumors, as used in the present invention. Those skilled in the art will be able to recognize and diagnose advanced solid tumors in a patient.

“Tumor burden” also referred to as “tumor load”, refers to the total amount of tumor material distributed throughout the body. Tumor burden refers to the total number of cancer cells or the total size of tumor(s), throughout the body, including lymph nodes and bone narrow. Tumor burden can be determined by a variety of methods known in the art, such as, e.g. by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

“Individual response” or “response” can be assessed using any endpoint indicating a benefit to the individual, including, without limitation, (1) inhibition, to some extent, of disease progression (e.g., cancer progression), including slowing down or complete arrest; (2) a reduction in tumor size; (3) inhibition (i.e., reduction, slowing down, or complete stopping) of cancer cell infiltration into adjacent peripheral organs and/or tissues; (4) inhibition (i.e. reduction, slowing down, or complete stopping) of metastasis; (5) relief, to some extent, of one or more symptoms associated with the disease or disorder (e.g., cancer); (6) increase or extension in the length of survival, including overall survival and progression free survival; and/or (7) decreased mortality at a given point of time following treatment.

An “effective response” of a patient or a patient's “responsiveness” to treatment with a medicament and similar wording refers to the clinical or therapeutic benefit imparted to a patient at risk for, or suffering from, a disease or disorder, such as cancer. In one embodiment, such benefit includes any one or more of: extending survival (including overall survival and/or progression-free survival); resulting in an objective response (including a complete response or a partial response); resulting in a complete response; resulting in a sustained response; or improving signs or symptoms of cancer.

An “objective response” or “OR” refers to a measurable response, including complete response (CR) or partial response (PR). An “objective response rate” (ORR) refers to the proportion of patients with tumor size reduction of a predefined amount and for a minimum time period. Generally, ORR refers to the sum of complete response (CR) rate and partial response (PR) rate.

“Complete response” or “CR” as used herein means the disappearance of all signs of cancer (e.g., disappearance of all target lesions) in response to treatment. This does not always mean the cancer has been cured.

As used herein, “partial response” or “PR” refers to a decrease in the size of one or more tumors or lesions, or in the extent of cancer in the body, in response to treatment. For example, in some embodiments, PR refers to at least a 30% decrease in the sum of the longest diameters (SLD) of target lesions, taking as reference the baseline SLD.

“Sustained response” refers to the sustained effect on reducing tumor growth after cessation of a treatment. For example, the tumor size may be the same size or smaller as compared to the size at the beginning of the medicament administration phase. In some embodiments, the sustained response has a duration of at least the same as the treatment duration, at least 1.5×, 2×, 2.5×, or 3× length of the treatment duration, or longer.

As used herein, “progression-free survival” (PFS) refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “overall survival” (OS) refers to the percentage of individuals in a group who are likely to be alive after a particular duration of time.

“Duration of Response” for purposes of the present invention means the time from documentation of tumor model growth inhibition due to drug treatment to the time of acquisition of a restored growth rate similar to pretreatment growth rate.

By “extending survival” is meant increasing overall or progression-free survival in a treated patient relative to an untreated patient (i.e. relative to a patient not treated with the medicament).

The term “synergy” or “synergistic” is used herein to mean that the effect of the combination of the two therapeutic agents of the combination therapy is greater than the sum of the effect of each agent when administered alone. A “synergistic amount” or “synergistically effective amount” is an amount of the combination of the two combination partners that results in a synergistic effect, as “synergistic” is defined herein. Determining a synergistic interaction between two combination partners, the optimum range for the effect and absolute dose ranges of each component for the effect may be definitively measured by administration of the combination partners over different w/w (weight per weight) ratio ranges and doses to patients in need of treatment. However, the observation of synergy in in vitro models or in vivo models can be predictive of the effect in humans and other species and in vitro models or in vivo models exist, as described herein, to measure a synergistic effect and the results of such studies can also be used to predict effective dose and plasma concentration ratio ranges and the absolute doses and plasma concentrations required in humans and other species by the application of pharmacokinetic/pharmacodynamic methods. For example, art-accepted in vitro and animal models of cancers described herein are known in the art and are described in the Examples. Exemplary synergistic effects include, but are not limited to, enhanced therapeutic efficacy, decreased dosage at equal or increased level of efficacy, reduced or delayed development of drug resistance, and simultaneous enhancement or equal therapeutic actions and reduction of unwanted side effects.

For example, a synergistic ratio of two therapeutic agents can be identified by determining a synergistic effect in an art-accepted in vitro (e.g., cancer cell line) or in vivo (animal model) model of any of the cancers described herein. Non-limiting examples of cancer cell lines and in vivo animal models of the cancers described herein are described in the Examples. Additional examples of art-accepted cancer cell lines and in vivo animal models are known in the art.

In some embodiments, “synergistic effect” as used herein refers to combination of Compound 1 or a pharmaceutically acceptable salt thereof, and a PD-1 inhibitor or a PD-L1 inhibitor producing an effect, for example, any of the beneficial or desired results including clinical results as described herein, for example slowing the symptomatic progression of a proliferative disease, particularly cancer, or symptoms thereof, which is greater than the sum of effect observed when Compound 1 or a pharmaceutically acceptable salt thereof and the PD-1 inhibitor or PD-L1 inhibitor are administered alone.

In some embodiments, the methods provided herein can result in about a 1% to 99% (e.g., 1% to 98%, 1% to 95%, 1% to 90%, 1 to 85%, 1 to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 2% to 99%, 2% to 90%, 2% to 85%, 2% to 80%, 2% to 75%, 2% to 70%, 2% to 65%, 2% to 60%, 2% to 55%, 2% to 50%, 2% to 45%, 2% to 40%, 2% to 35%, 2% to 30%, 2% to 25%, 2% to 20%, 2% to 15%, 2% to 10%, 2% to 5%, 4% to 99%, 4% to 95%, 4% to 90%, 4% to 85%, 4% to 80%, 4% to 75%, 4% to 70%, 4% to 65%, 4% to 60%, 4% to 55%, 4% to 50%, 4% to 45%, 4% to 40%, 4% to 35%, 4% to 30%, 4% to 25%, 4% to 20%, 4% to 15%, 4% to 10%, 6% to 99%, 6% to 95%, 6% to 90%, 6% to 85%, 6% to 80%, 6% to 75%, 6% to 70%, 6% to 65%, 6% to 60%, 6% to 55%, 6% to 50%, 6% to 45%, 6% to 40%, 6% to 35%, 6% to 30%, 6% to 25%, 6% to 20%, 6% to 15%, 6% to 10%, 8% to 99%, 8% to 95%, 8% to 90%, 8% to 85%, 8% to 80%, 8% to 75%, 8% to 70%, 8% to 65%, 8% to 60%, 8% to 55%, 8% to 50%, 8% to 45%, 8% to 40%, 8% to 35%, 8% to 30%, 8% to 25%, 8% to 20%, 8% to 15%, 10% to 99%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, 10% to 40%, 10% to 35%, 10% to 30%, 10% to 25%, 10% to 20%, 10% to 15%, 15% to 99%, 15% to 95%, 15% to 90%, 15% to 85%, 15% to 80%, 15% to 75%, 15% to 70%, 15% to 65%, 15% to 60%, 15% to 55%, 15% to 50%, 15% to 55%, 15% to 50%, 15% to 45%, 15% to 40%, 15% to 35%, 15% to 30%, 15% to 25%, 15% to 20%, 20% to 99%, 20% to 95%, 20% to 90%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 25% to 99%, 25% to 95%, 25% to 90%, 25% to 85%, 25% to 80%, 25% to 75%, 25% to 70%, 25% to 65%, 25% to 60%, 25% to 55%, 25% to 50%, 25% to 45%, 25% to 40%, 25% to 35%, 25% to 30%, 30% to 99%, 30% to 95%, 30% to 90%, 30% to 85%, 30% to 80%, 30% to 75%, 30% to 70%, 30% to 65%, 30% to 60%, 30% to 55%, 30% to 50%, 30% to 45%, 30% to 40%, 30% to 35%, 35% to 99%, 35% to 95%, 35% to 90%, 35% to 85%, 35% to 80%, 35% to 75%, 35% to 70%, 35% to 65%, 35% to 60%, 35% to 55%, 35% to 50%, 35% to 45%, 35% to 40%, 40% to 99%, 40% to 95%, 40% to 90%, 40% to 85%, 40% to 80%, 40% to 75%, 40% to 70%, 40% to 65%, 40% to 60%, 40% to 55%, 40% to 60%, 40% to 55%, 40% to 50%, 40% to 45%, 45% to 99%, 45% to 95%, 45% to 95%, 45% to 90%, 45% to 85%, 45% to 80%, 45% to 75%, 45% to 70%, 45% to 65%, 45% to 60%, 45% to 55%, 45% to 50%, 50% to 99%, 50% to 95%, 50% to 90%, 50% to 85%, 50% to 80%, 50% to 75%, 50% to 70%, 50% to 65%, 50% to 60%, 50% to 55%, 55% to 99%, 55% to 95%, 55% to 90%, 55% to 85%, 55% to 80%, 55% to 75%, 55% to 70%, 55% to 65%, 55% to 60%, 60% to 99%, 60% to 95%, 60% to 90%, 60% to 85%, 60% to 80%, 60% to 75%, 60% to 70%, 60% to 65%, 65% to 99%, 60% to 95%, 60% to 90%, 60% to 85%, 60% to 80%, 60% to 75%, 60% to 70%, 60% to 65%, 70% to 99%, 70% to 95%, 70% to 90%, 70% to 85%, 70% to 80%, 70% to 75%, 75% to 99%, 75% to 95%, 75% to 90%, 75% to 85%, 75% to 80%, 80% to 99%, 80% to 95%, 80% to 90%, 80% to 85%, 85% to 99%, 85% to 95%, 85% to 90%, 90% to 99%, 90% to 95%, or 95% to 100%) reduction in the volume of one or more solid tumors in a patient following treatment with the combination therapy for a period of time between about 1 day and 2 years (e.g., between 1 day and 22 months, between 1 day and 20 months, between 1 day and 18 months, between 1 day and 16 months, between 1 day and 14 months, between 1 day and 12 months, between 1 day and 10 months, between 1 day and 9 months, between 1 day and 8 months, between 1 day and 7 months, between 1 day and 6 months, between 1 day and 5 months, between 1 day and 4 months, between 1 day and 3 months, between 1 day and 2 months, between 1 day and 1 month, between one week and 2 years, between 1 week and 22 months, between 1 week and 20 months, between 1 week and 18 months, between 1 week and 16 months, between 1 week and 14 months, between 1 week and 12 months, between 1 week and 10 months, between 1 week and 9 months, between 1 week and 8 months, between 1 week and 7 months, between 1 week and 6 months, between 1 week and 5 months, between 1 week and 4 months, between 1 week and 3 months, between 1 week and 2 months, between 1 week and 1 month, between 2 weeks and 2 years, between 2 weeks and 22 months, between 2 weeks and 20 months, between 2 weeks and 18 months, between 2 weeks and 16 months, between 2 weeks and 14 months, between 2 weeks and 12 months, between 2 weeks and 10 months, between 2 weeks and 9 months, between 2 weeks and 8 months, between 2 weeks and 7 months, between 2 weeks and 6 months, between 2 weeks and 5 months, between 2 weeks and 4 months, between 2 weeks and 3 months, between 2 weeks and 2 months, between 2 weeks and 1 month, between 1 month and 2 years, between 1 month and 22 months, between 1 month and 20 months, between 1 month and 18 months, between 1 month and 16 months, between 1 month and 14 months, between 1 month and 12 months, between 1 month and 10 months, between 1 month and 9 months, between 1 month and 8 months, between 1 month and 7 months, between 1 month and 6 months, between 1 month and 6 months, between 1 month and 5 months, between 1 month and 4 months, between 1 month and 3 months, between 1 month and 2 months, between 2 months and 2 years, between 2 months and 22 months, between 2 months and 20 months, between 2 months and 18 months, between 2 months and 16 months, between 2 months and 14 months, between 2 months and 12 months, between 2 months and 10 months, between 2 months and 9 months, between 2 months and 8 months, between 2 months and 7 months, between 2 months and 6 months, or between 2 months and 5 months, between 2 months and 4 months, between 3 months and 2 years, between 3 months and 22 months, between 3 months and 20 months, between 3 months and 18 months, between 3 months and 16 months, between 3 months and 14 months, between 3 months and 12 months, between 3 months and 10 months, between 3 months and 8 months, between 3 months and 6 months, between 4 months and 2 years, between 4 months and 22 months, between 4 months and 20 months, between 4 months and 18 months, between 4 months and 16 months, between 4 months and 14 months, between 4 months and 12 months, between 4 months and 10 months, between 4 months and 8 months, between 4 months and 6 months, between 6 months and 2 years, between 6 months and 22 months, between 6 months and 20 months, between 6 months and 18 months, between 6 months and 16 months, between 6 months and 14 months, between 6 months and 12 months, between 6 months and 10 months, or between 6 months and 8 months) (e.g., as compared to the size of the one or more solid tumors in the patient prior to treatment).

In some embodiments, any of the methods described herein can provide for about a 1% to 99% (e.g., 1% to 98%, 1% to 95%, 1% to 90%, 1 to 85%, 1 to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 2% to 99%, 2% to 90%, 2% to 85%, 2% to 80%, 2% to 75%, 2% to 70%, 2% to 65%, 2% to 60%, 2% to 55%, 2% to 50%, 2% to 45%, 2% to 40%, 2% to 35%, 2% to 30%, 2% to 25%, 2% to 20%, 2% to 15%, 2% to 10%, 2% to 5%, 4% to 99%, 4% to 95%, 4% to 90%, 4% to 85%, 4% to 80%, 4% to 75%, 4% to 70%, 4% to 65%, 4% to 60%, 4% to 55%, 4% to 50%, 4% to 45%, 4% to 40%, 4% to 35%, 4% to 30%, 4% to 25%, 4% to 20%, 4% to 15%, 4% to 10%, 6% to 99%, 6% to 95%, 6% to 90%, 6% to 85%, 6% to 80%, 6% to 75%, 6% to 70%, 6% to 65%, 6% to 60%, 6% to 55%, 6% to 50%, 6% to 45%, 6% to 40%, 6% to 35%, 6% to 30%, 6% to 25%, 6% to 20%, 6% to 15%, 6% to 10%, 8% to 99%, 8% to 95%, 8% to 90%, 8% to 85%, 8% to 80%, 8% to 75%, 8% to 70%, 8% to 65%, 8% to 60%, 8% to 55%, 8% to 50%, 8% to 45%, 8% to 40%, 8% to 35%, 8% to 30%, 8% to 25%, 8% to 20%, 8% to 15%, 10% to 99%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, 10% to 40%, 10% to 35%, 10% to 30%, 10% to 25%, 10% to 20%, 10% to 15%, 15% to 99%, 15% to 95%, 15% to 90%, 15% to 85%, 15% to 80%, 15% to 75%, 15% to 70%, 15% to 65%, 15% to 60%, 15% to 55%, 15% to 50%, 15% to 55%, 15% to 50%, 15% to 45%, 15% to 40%, 15% to 35%, 15% to 30%, 15% to 25%, 15% to 20%, 20% to 99%, 20% to 95%, 20% to 90%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 25% to 99%, 25% to 95%, 25% to 90%, 25% to 85%, 25% to 80%, 25% to 75%, 25% to 70%, 25% to 65%, 25% to 60%, 25% to 55%, 25% to 50%, 25% to 45%, 25% to 40%, 25% to 35%, 25% to 30%, 30% to 99%, 30% to 95%, 30% to 90%, 30% to 85%, 30% to 80%, 30% to 75%, 30% to 70%, 30% to 65%, 30% to 60%, 30% to 55%, 30% to 50%, 30% to 45%, 30% to 40%, 30% to 35%, 35% to 99%, 35% to 95%, 35% to 90%, 35% to 85%, 35% to 80%, 35% to 75%, 35% to 70%, 35% to 65%, 35% to 60%, 35% to 55%, 35% to 50%, 35% to 45%, 35% to 40%, 40% to 99%, 40% to 95%, 40% to 90%, 40% to 85%, 40% to 80%, 40% to 75%, 40% to 70%, 40% to 65%, 40% to 60%, 40% to 55%, 40% to 60%, 40% to 55%, 40% to 50%, 40% to 45%, 45% to 99%, 45% to 95%, 45% to 95%, 45% to 90%, 45% to 85%, 45% to 80%, 45% to 75%, 45% to 70%, 45% to 65%, 45% to 60%, 45% to 55%, 45% to 50%, 50% to 99%, 50% to 95%, 50% to 90%, 50% to 85%, 50% to 80%, 50% to 75%, 50% to 70%, 50% to 65%, 50% to 60%, 50% to 55%, 55% to 99%, 55% to 95%, 55% to 90%, 55% to 85%, 55% to 80%, 55% to 75%, 55% to 70%, 55% to 65%, 55% to 60%, 60% to 99%, 60% to 95%, 60% to 90%, 60% to 85%, 60% to 80%, 60% to 75%, 60% to 70%, 60% to 65%, 65% to 99%, 60% to 95%, 60% to 90%, 60% to 85%, 60% to 80%, 60% to 75%, 60% to 70%, 60% to 65%, 70% to 99%, 70% to 95%, 70% to 90%, 70% to 85%, 70% to 80%, 70% to 75%, 75% to 99%, 75% to 95%, 75% to 90%, 75% to 85%, 75% to 80%, 80% to 99%, 80% to 95%, 80% to 90%, 80% to 85%, 85% to 99%, 85% to 95%, 85% to 90%, 90% to 99%, 90% to 95%, or 95% to 100%) reduction in the risk of developing a metastasis or the risk of developing an additional metastasis in a patient having a cancer.

The phrase “time of survival” means the length of time between the identification or diagnosis of cancer (e.g., any of the cancers described herein) in a mammal by a medical professional and the time of death of the mammal (caused by the cancer). Methods of increasing the time of survival in a mammal having a cancer are described herein.

In some embodiments, any of the methods described herein can result in an increase (e.g., about a 1% to 400%, 1% to 380%, 1% to 360%, 1% to 340%, 1% to 320%, 1% to 300%, 1% to 280%, 1% to 260%, 1% to 240%, 1% to 220%, 1% to 200%, 1% to 180%, 1% to 160%, 1% to 140%, 1% to 120%, 1% to 100%, 1% to 95%, 1% to 90%, 1% to 85%, 1% to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 5% to 400%, 5% to 380%, 5% to 360%, 5% to 340%, 5% to 320%, 5% to 300%, 5% to 280%, 5% to 260%, 5% to 240%, 5% to 220%, 5% to 200%, 5% to 180%, 5% to 160%, 5% to 140%, 5% to 120%, 5% to 100%, 5% to 90%, 5% to 80%, 5% to 70%, 5% to 60%, 5% to 50%, 5% to 40%, 5% to 30%, 5% to 20%, 5% to 10%, 10% to 400%, 10% to 380%, 10% to 360%, 10% to 340%, 10% to 320%, 10% to 300%, 10% to 280%, 10% to 260%, 10% to 240%, 10% to 220%, 10% to 200%, 10% to 180%, 10% to 160%, 10% to 140%, 10% to 120%, 10% to 100%, 10% to 90%, 10% to 80%, 10% to 70%, 10% to 60%, 10% to 50%, 10% to 40%, 10% to 30%, 10% to 20%, 20% to 400%, 20% to 380%, 20% to 360%, 20% to 340%, 20% to 320%, 20% to 300%, 20% to 280%, 20% to 260%, 20% to 240%, 20% to 220%, 20% to 200%, 20% to 180%, 20% to 160%, 20% to 140%, 20% to 120%, 20% to 100%, 20% to 90%, 20% to 80%, 20% to 70%, 20% to 60%, 20% to 50%, 20% to 40%, 20% to 30%, 30% to 400%, 30% to 380%, 30% to 360%, 30% to 340%, 30% to 320%, 30% to 300%, 30% to 280%, 30% to 260%, 30% to 240%, 30% to 220%, 30% to 200%, 30% to 180%, 30% to 160%, 30% to 140%, 30% to 120%, 30% to 100%, 30% to 90%, 30% to 80%, 30% to 70%, 30% to 60%, 30% to 50%, 30% to 40%, 40% to 400%, 40% to 380%, 40% to 360%, 40% to 340%, 40% to 320%, 40% to 300%, 40% to 280%, 40% to 260%, 40% to 240%, 40% to 220%, 40% to 200%, 40% to 180%, 40% to 160%, 40% to 140%, 40% to 120%, 40% to 100%, 40% to 90%, 40% to 80%, 40% to 70%, 40% to 60%, 40% to 50%, 50% to 400%, 50% to 380%, 50% to 360%, 50% to 340%, 50% to 320%, 50% to 300%, 50% to 280%, 50% to 260%, 50% to 240%, 50% to 220%, 50% to 200%, 50% to 180%, 50% to 160%, 50% to 140%, 50% to 140%, 50% to 120%, 50% to 100%, 50% to 90%, 50% to 80%, 50% to 70%, 50% to 60%, 60% to 400%, 60% to 380%, 60% to 360%, 60% to 340%, 60% to 320%, 60% to 300%, 60% to 280%, 60% to 260%, 60% to 240%, 60% to 220%, 60% to 200%, 60% to 180%, 60% to 160%, 60% to 140%, 60% to 120%, 60% to 100%, 60% to 90%, 60% to 80%, 60% to 70%, 70% to 400%, 70% to 380%, 70% to 360%, 70% to 340%, 70% to 320%, 70% to 300%, 70% to 280%, 70% to 260%, 70% to 240%, 70% to 220%, 70% to 200%, 70% to 180%, 70% to 160%, 70% to 140%, 70% to 120%, to 100%, 70% to 90%, 70% to 80%, 80% to 400%, 80% to 380%, 80% to 360%, 80% to 340%, 80% to 320%, 80% to 300%, 80% to 280%, 80% to 260%, 80% to 240%, 80% to 220%, 80% to 200%, 80% to 180%, 80% to 160%, 80% to 140%, 80% to 120%, 80% to 100%, 80% to 90%, 90% to 400%, 90% to 380%, 90% to 360%, 90% to 340%, 90% to 320%, 90% to 300%, 90% to 280%, 90% to 260%, 90% to 240%, 90% to 220%, 90% to 200%, 90% to 180%, 90% to 160%, 90% to 140%, 90% to 120%, 90% to 100%, 100% to 400%, 100% to 380%, 100% to 360%, 100% to 340%, 100% to 320%, 100% to 300%, 100% to 280%, 100% to 260%, 100% to 240%, 100% to 220%, 100% to 200%, 100% to 180%, 100% to 160%, 100% to 140%, 100% to 120%, 120% to 400%, 120% to 380%, 120% to 360%, 120% to 340%, 120% to 320%, 120% to 300%, 120% to 280%, 120% to 260%, 120% to 240%, 120% to 220%, 120% to 200%, 120% to 180%, 120% to 160%, 120% to 140%, 140% to 400%, 140% to 380%, 140% to 360%, 140% to 340%, 140% to 320%, 140% to 300%, 140% to 280%, 140% to 260%, 140% to 240%, 140% to 220%, 140% to 200%, 140% to 180%, 140% to 160%, 160% to 400%, 160% to 380%, 160% to 360%, 160% to 340%, 160% to 320%, 160% to 300%, 160% to 280%, 160% to 260%, 160% to 240%, 160% to 220%, 160% to 200%, 160% to 180%, 180% to 400%, 180% to 380%, 180% to 360%, 180% to 340%, 180% to 320%, 180% to 300%, 180% to 280%, 180% to 260%, 180% to 240%, 180% to 220%, 180% to 200%, 200% to 400%, 200% to 380%, 200% to 360%, 200% to 340%, 200% to 320%, 200% to 300%, 200% to 280%, 200% to 260%, 200% to 240%, 200% to 220%, 220% to 400%, 220% to 380%, 220% to 360%, 220% to 340%, 220% to 320%, 220% to 300%, 220% to 280%, 220% to 260%, 220% to 240%, 240% to 400%, 240% to 380%, 240% to 360%, 240% to 340%, 240% to 320%, 240% to 300%, 240% to 280%, 240% to 260%, 260% to 400%, 260% to 380%, 260% to 360%, 260% to 340%, 260% to 320%, 260% to 300%, 260% to 280%, 280% to 400%, 280% to 380%, 280% to 360%, 280% to 340%, 280% to 320%, 280% to 300%, 300% to 400%, 300% to 380%, 300% to 360%, 300% to 340%, or 300% to 320%) in the time of survival of the patient (e.g., as compared to a patient having a similar cancer and administered a different treatment or not receiving a treatment).

As used herein, the term “cytokine” refers generically to proteins released by one cell population that act on another cell as intercellular mediators or have an autocrine effect on the cells producing the proteins. Examples of such cytokines include lymphokines; monokines; interleukins (“ILs”) such as IL-1, IL-1a, IL-2, IL-3, IL-4, IL-5, IL-6, IL-7, IL-8, IL-9, IL10, IL-1 1, IL-12, IL-13, IL-15, IL-17A-F, IL-18 to IL-29 (such as IL-23), IL-31, including PROLEUKIN® rIL-2; a tumor-necrosis factor such as TNF-a or TNF-β, TGF-I-3; and other polypeptide factors including leukemia inhibitory factor (“LIF”), ciliary neurotrophic factor (“CNTF”), CNTF-like cytokine (“CLC”), cardiotrophin (“CT”), and kit ligand (“L”).

As used herein, the term “chemokine” refers to soluble factors (e.g., cytokines) that have the ability to selectively induce chemotaxis and activation of leukocytes. They also trigger processes of angiogenesis, inflammation, wound healing, and tumorigenesis. Example chemokines include IL-8, a human homolog of murine keratinocyte chemoattractant (KC).

### Methods, Uses, and Medicaments

In one embodiment, provided herein is a method for treating cancer, comprising administering to a patient in need thereof, over a period of time, a therapeutically effective amount of Compound 1 or a pharmaceutically acceptable salt thereof as a monotherapy, wherein Compound 1 or a pharmaceutically acceptable salt thereof is administered according to an intermittent dosing schedule of at least one dosing cycle, wherein each dosing cycle comprises (a) a dosing period during which said Compound 1 or a pharmaceutically acceptable salt thereof is administered, and (b) a resting period during which said Compound 1 or a pharmaceutically acceptable salt thereof is not administered. In one embodiment, the dosing cycle is 21 days. In one embodiment, the dosing cycle is 28 days.

In one embodiment, provided herein is a method for treating cancer comprising administering to a patient in need thereof, over a period of time, a therapeutically effective amount of Compound 1 or a pharmaceutically acceptable salt thereof as a monotherapy, wherein said Compound 1 or a pharmaceutically acceptable salt thereof is administered according to an intermittent dosing schedule of at least one 21-day dosing cycle, wherein each dosing cycle comprises (a) a dosing period during which said Compound 1 or a pharmaceutically acceptable salt thereof is administered on days 1-14, and (b) a resting period during which said Compound 1 or a pharmaceutically acceptable salt thereof is not administered, wherein the resting period is days 15-21.

In one embodiment, provided herein is a method for treating cancer comprising administering to a patient in need thereof, over a period of time, a therapeutically effective amount of Compound 1 or a pharmaceutically acceptable salt thereof as a monotherapy, wherein said Compound 1 or a pharmaceutically acceptable salt thereof is administered according to an intermittent dosing schedule of at least one 28-day dosing cycle, wherein each dosing cycle comprises (a) a dosing period during which said Compound 1 or a pharmaceutically acceptable salt thereof is administered on days 1-14, and (b) a resting period during which said Compound 1 or a pharmaceutically acceptable salt thereof is not administered, wherein the resting period is days 15-28.

In one embodiment, provided herein is a method for treating cancer comprising administering to a patient in need thereof, over a period of time, a therapeutically effective amount of Compound 1 or a pharmaceutically acceptable salt thereof as a monotherapy, wherein said Compound 1 or a pharmaceutically acceptable salt thereof is administered according to an intermittent dosing schedule of at least one 28-day dosing cycle, wherein each dosing cycle comprises (a) a dosing period during which said Compound 1 or a pharmaceutically acceptable salt thereof is administered on days 1-7 and days 15-21, and (b) a resting period during which said Compound 1 or a pharmaceutically acceptable salt thereof is not administered, wherein the resting period is days 8-14 and 22-28.

In one embodiment of any of the methods disclosed herein for administering Compound 1 or a pharmaceutically acceptable salt thereof as a monotherapy on an intermittent dosing schedule, Compound 1 or a pharmaceutically acceptable salt thereof is administered orally.

In one embodiment of any of the methods disclosed herein for administering Compound 1 or a pharmaceutically acceptable salt thereof as a monotherapy on an intermittent dosing schedule, Compound 1 or a pharmaceutically acceptable salt thereof is administered once a day (QD). In one embodiment of any of the methods disclosed herein for administering Compound 1 or a pharmaceutically acceptable salt thereof on an intermittent dosing schedule, a single dose of Compound 1 or a pharmaceutically acceptable salt thereof is divided into two sub-doses and Compound 1 or a pharmaceutically acceptable salt thereof is administered twice a day (BID). In one embodiment of a daily dose of Compound 1 or a pharmaceutically acceptable salt thereof wherein the doses are divided into a first and second dose, the second dose of Compound 1 or a pharmaceutically acceptable salt thereof is administered at approximately the same time as the first dose of Compound 1 or a pharmaceutically acceptable salt thereof. In one embodiment of a daily dose of Compound 1 or a pharmaceutically acceptable salt thereof wherein the doses are divided into a first and second dose, the second dose of Compound 1 or a pharmaceutically acceptable salt thereof is administered about 12 hours after the first dose of Compound 1 or a pharmaceutically acceptable salt thereof.

In some embodiment of any of the methods disclosed herein for administering Compound 1 or a pharmaceutically acceptable salt thereof as a monotherapy on an intermittent dosing schedule, Compound 1 or a pharmaceutically acceptable salt thereof is formulated as a capsule or tablet for oral administration. In some embodiments, a capsule or tablet comprises 25 mg, 50 mg, 100 mg, 200 mg or 300 mg of Compound 1 or a pharmaceutically acceptable salt thereof. In some embodiments, a capsule or tablet comprises 25 mg, 50 mg, 75 mg, 100 mg, 150 mg, 200 mg, 250 mg or 300 mg of Compound 1 or a pharmaceutically acceptable salt thereof.

In some embodiment of any of the methods disclosed herein for administering Compound 1 or a pharmaceutically acceptable salt thereof as a monotherapy on an intermittent dosing schedule, the patient is dosed daily with 25 mg, 50 mg, 100 mg, 200 mg or 300 mg of Compound 1 or a pharmaceutically acceptable salt thereof during each dosing period. Compound 1 or a pharmaceutically acceptable salt thereof may be administered as a single dose of 25 mg, 50 mg, 100 mg, 200 mg or 300 mg or as divided doses which may be administered at approximately the same time, or at timed intervals (e.g., as two sub-doses administered 12 hours apart).

In one embodiment of any of the methods disclosed herein for administering Compound 1 or a pharmaceutically acceptable salt thereof in combination with a PD-1 or PD-L1 inhibitor on an intermittent dosing schedule, Compound 1 or a pharmaceutically acceptable salt thereof is formulated as a tablet or capsule. In some embodiments, a capsule or tablet comprises 25 mg, 50 mg, 75 mg, 100 mg, 150 mg, 200 mg, 250 mg or 300 mg of Compound 1 or a pharmaceutically acceptable salt thereof.

In some embodiment of any of the methods disclosed herein for administering Compound 1 or a pharmaceutically acceptable salt thereof in combination with a PD-1 or PD-L1 inhibitor on an intermittent dosing schedule, the patient is dosed daily with 25 mg, 50 mg, 100 mg, 200 mg or 300 mg of Compound 1 or a pharmaceutically acceptable salt thereof during each dosing period. Compound 1 or a pharmaceutically acceptable salt thereof may be administered as a single dose of 25 mg, 50 mg, 100 mg, 200 mg or 300 mg or as divided doses which may be administered at approximately the same time, or at timed intervals (e.g., as two sub-doses administered 12 hours apart).

In one embodiment, provided herein is a method of treating a proliferative disease, including cancer, which comprises administering, during a period of time, a combination therapy comprising Compound 1 or a pharmaceutically acceptable salt thereof, and a PD-1 inhibitor or a PD-L1 inhibitor, to a patient in need thereof, wherein Compound 1 or a pharmaceutically acceptable salt thereof is administered on an intermittent dosing schedule and wherein the Compound 1 or a pharmaceutically acceptable salt thereof, and the PD-1 inhibitor or PD-L1 inhibitor are administered in jointly therapeutically effective amounts (for example in synergistically effective amounts). The individual components of this combination therapy of the invention may be administered separately at different times and in any order during the dosing period.

The term “jointly therapeutically effective amount” as used herein means when the therapeutic agents of a combination described herein are given to the patient simultaneously or separately (e.g., in a chronologically staggered manner, for example a sequence-specific manner) in such time intervals that they show an interaction (e.g., a joint therapeutic effect, for example a synergistic effect). Whether this is the case can, inter alia, be determined by following the blood levels and showing that the combination components are present in the blood of the human to be treated at least during certain time intervals.

In one embodiment, provided herein is a method for treating cancer comprising administering to a patient in need thereof, over a period of time, a therapeutically effective amount of Compound 1 or a pharmaceutically acceptable salt thereof and a PD-1 inhibitor according to an intermittent dosing schedule of at least one dosing cycle, wherein each dosing cycle comprises (a) a dosing period during which said Compound 1 or a pharmaceutically acceptable salt thereof and said PD-1 inhibitor are administered, and (b) a resting period during which said Compound 1 or a pharmaceutically acceptable salt thereof and said PD-1 inhibitor are not administered. In one embodiment, the dosing cycle is 28 days. In one embodiment, the dosing cycle is 21 days.

In one embodiment, provided herein is a method for treating cancer comprising administering to a patient in need thereof, over a period of time, a therapeutically effective amount of Compound 1 or a pharmaceutically acceptable salt thereof and a PD-1 inhibitor according to an intermittent dosing schedule of at least one dosing cycle, wherein each dosing cycle is a 28-day cycle and comprises (a) a dosing period during which said Compound 1 or a pharmaceutically acceptable salt thereof is administered on days 1 through 7 and said PD-1 inhibitor is administered on day 1 and day 15, and (b) a resting period during which said Compound 1 or a pharmaceutically acceptable salt thereof and said PD-1 inhibitor are not administered, wherein the resting period is days 16 through 28. In one embodiment, the PD-1 inhibitor is nivolumab or a biosimilar thereof. In one embodiment, the PD-1 inhibitor is sasanlimab or a biosimilar thereof.

In one embodiment, provided herein is a method for treating cancer comprising administering to a patient in need thereof, over a period of time, a therapeutically effective amount of Compound 1 or a pharmaceutically acceptable salt thereof and a PD-1 inhibitor according to an intermittent dosing schedule of at least one dosing cycle, wherein each dosing cycle is a 28-day cycle and comprises (a) a dosing period during which said Compound 1 or a pharmaceutically acceptable salt thereof is administered on days 1 through 14 and said PD-1 inhibitor is administered on day 1 and day 15, and (b) a resting period during which said Compound 1 or a pharmaceutically acceptable salt thereof and said PD-1 inhibitor are not administered, wherein the resting period is days 16 through 28. In one embodiment, the PD-1 inhibitor is nivolumab or a biosimilar thereof. In one embodiment, the PD-1 inhibitor is sasanlimab or a biosimilar thereof.

In one embodiment, provided herein is a method for treating cancer comprising administering to a patient in need thereof, over a period of time, a therapeutically effective amount of Compound 1 or a pharmaceutically acceptable salt thereof and a PD-1 inhibitor according to an intermittent dosing schedule of at least one dosing cycle, wherein each dosing cycle is a 28-day cycle and comprises (a) a dosing period during which said Compound 1 or a pharmaceutically acceptable salt thereof is administered on days 1 through 21, and said PD-1 inhibitor is administered on day 1 and day 15, and (b) a resting period during which said Compound 1 or a pharmaceutically acceptable salt thereof and said PD-1 inhibitor are not administered, wherein the resting period is days 22 through 28. In one embodiment, the PD-1 inhibitor is nivolumab or a biosimilar thereof. In one embodiment, the PD-1 inhibitor is sasanlimab or a biosimilar thereof.

In one embodiment, provided herein is a method for treating cancer comprising administering to a patient in need thereof, over a period of time, a therapeutically effective amount of Compound 1 or a pharmaceutically acceptable salt thereof and a PD-1 inhibitor according to an intermittent dosing schedule of at least one dosing cycle, wherein each dosing cycle is a 28-day cycle and comprises (a) a dosing period during which said Compound 1 or a pharmaceutically acceptable salt thereof is administered on days 1 through 7 and days 15 through 21, and said PD-1 inhibitor is administered on day 1 and day 15, and (b) a resting period during which said Compound 1 or a pharmaceutically acceptable salt thereof and said PD-1 inhibitor are not administered, wherein the resting period is days 8 through 14 and 22 through 28. In one embodiment, the PD-1 inhibitor is nivolumab or a biosimilar thereof. In one embodiment, the PD-1 inhibitor is sasanlimab or a biosimilar thereof.

In any of the embodiments disclosed herein for treating cancer comprising administering to a patient in need thereof, over a period of time, a therapeutically effective amount of Compound 1 or a pharmaceutically acceptable salt thereof and a PD-1 inhibitor which is nivolumab according to an intermittent dosing schedule, nivolumab is administered intravenously at a dose of about 3 mg/kg or as a flat dose of about 240 mg over 30 minutes.

In one embodiment, provided herein is a method for treating cancer comprising administering to a patient in need thereof, over a period of time, a therapeutically effective amount of Compound 1 or a pharmaceutically acceptable salt thereof and a PD-1 inhibitor according to an intermittent dosing schedule of at least one dosing cycle, wherein each dosing cycle is a 21-day cycle and comprises (a) a dosing period during which said Compound 1 or a pharmaceutically acceptable salt thereof is administered on days 1 through 7 and said PD-1 inhibitor is administered on day 1, and (b) a resting period during which said Compound 1 or a pharmaceutically acceptable salt thereof and said PD-1 inhibitor are not administered, wherein the resting period is days 8 through 21. In one embodiment, the PD-1 inhibitor is pembrolizumab or a biosimilar thereof. In one embodiment, the PD-1 inhibitor is sasanlimab or a biosimilar thereof.

In one embodiment, provided herein is a method for treating cancer comprising administering to a patient in need thereof, over a period of time, a therapeutically effective amount of Compound 1 or a pharmaceutically acceptable salt thereof and a PD-1 inhibitor according to an intermittent dosing schedule of at least one dosing cycle, wherein each dosing cycle is a 21-day cycle and comprises (a) a dosing period during which said Compound 1 or a pharmaceutically acceptable salt thereof is administered on days 1 through 14 and said PD-1 inhibitor is administered on day 1, and (b) a resting period during which said Compound 1 or a pharmaceutically acceptable salt thereof and said PD-1 inhibitor are not administered, wherein the resting period is days 15 through 21. In one embodiment, the PD-1 inhibitor is pembrolizumab or a biosimilar thereof. In one embodiment, the PD-1 inhibitor is sasanlimab or a biosimilar thereof.

In any of the embodiments disclosed herein for treating cancer comprising administering to a patient in need thereof, over a period of time, a therapeutically effective amount of Compound 1 or a pharmaceutically acceptable salt thereof and a PD-1 inhibitor which is pembrolizumab according to an intermittent dosing schedule, pembrolizumab is administered intravenously at a dose of about 2 mg/kg or as a flat dose of about 200 mg over 30 minutes.

In any of the embodiments disclosed herein for treating cancer comprising administering to a patient in need thereof, over a period of time, a therapeutically effective amount of Compound 1 or a pharmaceutically acceptable salt thereof and a PD-1 inhibitor according to an intermittent dosing schedule, Compound 1 or a pharmaceutically acceptable salt thereof is formulated as a capsule or tablet for oral administration. In some embodiments, a capsule or tablet comprises 25 mg, 50 mg, 75 mg, 100 mg, 150 mg, 200 mg, 250 mg or 300 mg of Compound 1 or a pharmaceutically acceptable salt thereof. In some of said embodiments, Compound 1 or a pharmaceutically acceptable salt thereof is administered once a day during said dosing period. In some of said embodiments, a single dose of Compound 1 or a pharmaceutically acceptable salt thereof is divided into two sub-doses and Compound 1 or a pharmaceutically acceptable salt thereof is administered twice a day (BID). In one embodiment of a daily dose of Compound 1 or a pharmaceutically acceptable salt thereof wherein the doses are divided into a first and second dose, the second dose of Compound 1 or a pharmaceutically acceptable salt thereof is administered about 12 hours after the first dose of Compound 1 or a pharmaceutically acceptable salt thereof.

In one embodiment, provided herein is a method for treating cancer comprising administering to a patient in need thereof, over a period of time, a therapeutically effective amount of Compound 1 or a pharmaceutically acceptable salt thereof and a PD-L1 inhibitor according to an intermittent dosing schedule of at least one dosing cycle, wherein each dosing cycle comprises (a) a dosing period during which said Compound 1 or a pharmaceutically acceptable salt thereof and said PD-L1 inhibitor are administered, and (b) a resting period during which said Compound 1 or a pharmaceutically acceptable salt thereof and said PD-L1 inhibitor are not administered. In one embodiment, the dosing cycle is a 28-day cycle.

In one embodiment, provided herein is a method for treating cancer comprising administering to a patient in need thereof, over a period of time, a therapeutically effective amount of Compound 1 or a pharmaceutically acceptable salt thereof and a PD-L1 inhibitor according to an intermittent dosing schedule of at least one dosing cycle, wherein each dosing cycle is a 28-day cycle and comprises (a) a dosing period during which said Compound 1 or a pharmaceutically acceptable salt thereof is administered on days 1 through 7, and said PD-L1 inhibitor is administered on day 1 and day 15, and (b) a resting period during which said Compound 1 or a pharmaceutically acceptable salt thereof and said PD-L1 inhibitor are not administered, wherein the resting period is days 16 through 28. In one embodiment, the PD-L1 inhibitor is atezolizumab or a biosimilar thereof.

In one embodiment, provided herein is a method for treating cancer comprising administering to a patient in need thereof, over a period of time, a therapeutically effective amount of Compound 1 or a pharmaceutically acceptable salt thereof and a PD-L1 inhibitor according to an intermittent dosing schedule of at least one dosing cycle, wherein each dosing cycle is a 28-day cycle and comprises (a) a dosing period during which said Compound 1 or a pharmaceutically acceptable salt thereof is administered on days 1 through 14, and said PD-L1 inhibitor is administered on day 1 and day 15, and (b) a resting period during which said Compound 1 or a pharmaceutically acceptable salt thereof and said PD-L1 inhibitor are not administered, wherein the resting period is days 16 through 28. In one embodiment, the PD-L1 inhibitor is atezolizumab or a biosimilar thereof.

In one embodiment, provided herein is a method for treating cancer comprising administering to a patient in need thereof, over a period of time, a therapeutically effective amount of Compound 1 or a pharmaceutically acceptable salt thereof and a PD-L1 inhibitor according to an intermittent dosing schedule of at least one dosing cycle, wherein each dosing cycle is a 28-day cycle and comprises (a) a dosing period during which said Compound 1 or a pharmaceutically acceptable salt thereof is administered on days 1 through 21, and said PD-L1 inhibitor is administered on day 1 and day 15, and (b) a resting period during which said Compound 1 or a pharmaceutically acceptable salt thereof and said PD-L1 inhibitor are not administered, wherein the resting period is days 22 through 28. In one embodiment, the PD-L1 inhibitor is atezolizumab or a biosimilar thereof.

In one embodiment, provided herein is a method for treating cancer comprising administering to a patient in need thereof, over a period of time, a therapeutically effective amount of Compound 1 or a pharmaceutically acceptable salt thereof and a PD-L1 inhibitor according to an intermittent dosing schedule of at least one dosing cycle, wherein each dosing cycle is a 28-day cycle and comprises (a) a dosing period during which said Compound 1 or a pharmaceutically acceptable salt thereof is administered on days 1 through 7 and days 15 through 21, and said PD-L1 inhibitor is administered on day 1 and day 15, and (b) a resting period during which said Compound 1 or a pharmaceutically acceptable salt thereof and said PD-L1 inhibitor are not administered, wherein the resting period is days 8 through 14 and 22 through 28. In one embodiment, the PD-L1 inhibitor is atezolizumab or a biosimilar thereof.

In any of the embodiments disclosed herein for treating cancer comprising administering to a patient in need thereof, over a period of time, a therapeutically effective amount of Compound 1 or a pharmaceutically acceptable salt thereof and a PD-L1 inhibitor which is atezolizumab according to an intermittent dosing schedule, atezolizumab is administered intravenously at a dose of about 840 mg over 30 or 60 minutes.

In any of the embodiments disclosed herein for treating cancer comprising administering to a patient in need thereof, over a period of time, a therapeutically effective amount of Compound 1 or a pharmaceutically acceptable salt thereof and a PD-L1 inhibitor according to an intermittent dosing schedule, Compound 1 or a pharmaceutically acceptable salt thereof is formulated as a capsule or tablet for oral administration. In some embodiments, a capsule or tablet comprises 25 mg, 50 mg, 75 mg, 100 mg, 150 mg, 200 mg, 250 mg or 300 mg of Compound 1 or a pharmaceutically acceptable salt thereof. In some of said embodiments, Compound 1 or a pharmaceutically acceptable salt thereof is administered once a day during said dosing period. In some of said embodiments, a single dose of Compound 1 or a pharmaceutically acceptable salt thereof is divided into two sub-doses and Compound 1 or a pharmaceutically acceptable salt thereof is administered twice a day (BID). In one embodiment of a daily dose of Compound 1 or a pharmaceutically acceptable salt thereof wherein the doses are divided into a first and second dose, the second dose of Compound 1 or a pharmaceutically acceptable salt thereof is administered about 12 hours after the first dose of Compound 1 or a pharmaceutically acceptable salt thereof.

In one embodiment of any of the dosing schedules of a combination therapy as described herein, on days when the PD-1 or PD-L1 inhibitor is administered during the dosing period, the PD-1 or PD-L1 inhibitor is administered at least 30 minutes after the administration of a therapeutically effective amount of Compound 1 or a pharmaceutically acceptable salt thereof. As used herein, the phrase “at least 30 minutes after” means that the PD-1 inhibitor is administered during the dosing period at least 5 minutes, or at least 10 minutes, or at least 15 minutes, or at least 20 minutes, or at least 25 minutes, or at least 30 minutes, or at least 35 minutes, or at least 40 minutes, or at least 45 minutes, or at least 50 minutes, or at least 55 minutes, or at least 60 minutes, or at least 65 minutes, or at least 70 minutes, or at least 75 minutes, or at least 80 minutes, or at least 85 minutes, or at least 90 minutes after the administration of Compound 1 or a pharmaceutically acceptable salt thereof, during the dosing period.

In one embodiment of any of the dosing schedules of a combination therapy as described herein, on days when the PD-1 or PD-L1 inhibitor is administered, during the dosing period, the PD-1 or PD-L1 inhibitor is administered at least 30 minutes before the administration of a therapeutically effective amount of the therapeutically effective dose of Compound 1 or a pharmaceutically acceptable salt thereof, during the dosing period. As used herein, the phrase “at least 30 minutes after” means that the PD-1 or PD-L1 inhibitor is administered during the dosing period at least 5 minutes, or at least 10 minutes, or at least 15 minutes, or at least 20 minutes, or at least 25 minutes, or at least 30 minutes, or at least 35 minutes, or at least 40 minutes, or at least 45 minutes, or at least 50 minutes, or at least 55 minutes, or at least 60 minutes, or at least 65 minutes, or at least 70 minutes, or at least 75 minutes, or at least 80 minutes, or at least 85 minutes, or at least 90 minutes before administration of the dose of Compound 1 or a pharmaceutically acceptable salt thereof, during the dosing period.

In one embodiment, the dose of Compound 1 or a pharmaceutically acceptable salt thereof is escalated during the dosing period until the Maximum Tolerated Dosage is reached, and the PD-1 inhibitor is administered as a fixed dose, during the dosing period. Alternatively, Compound 1 or a pharmaceutically acceptable salt thereof may be administered as a fixed dose during the dosing period and the dose of the PD-1 inhibitor may be escalated until the Maximum Tolerated Dosage is reached, during the dosing period.

In one embodiment of any combination therapy described herein may further comprise administration of one or more pre-medications prior to the administration of the PD-1 or PD-L1 inhibitor, during the dosing period. In one embodiment, the one or more pre-medication(s) is administered during the dosing period no sooner than 1 hour after administration of Compound 1 or a pharmaceutically acceptable salt thereof. In one embodiment, the one or more premedication(s) is administered 30-60 minutes prior to the administration of the PD-1 or PD-L1 inhibitor, during the dosing period. In one embodiment, the one or more premedication(s) is administered 30 minutes prior administration of the PD-1 or PD-L1 inhibitor, during the dosing period. In one embodiment, the one or more pre-medications is selected from one or more of a H₁ antagonist (e.g., antihistamines such as diphenhydramine) and acetaminophen.

In one embodiment of any of the dosing cycles disclosed herein, at least 2 dosing cycles are completed. In one embodiment of any of the dosing cycles disclosed herein, at least 5 dosing cycles are completed. In one embodiment of any of the dosing cycles disclosed herein, at least 10 dosing cycles are completed. In one embodiment of any of the dosing cycles disclosed herein, at least 20 dosing cycles are completed.

In some embodiments, provided herein is the use of Compound 1 or a pharmaceutically acceptable salt thereof, in the manufacture of a medicament for the treatment of cancer, wherein the medicament is administered as a monotherapy according to an intermittent dosing schedule of at least one dosing cycle, wherein each dosing cycle comprises (a) a dosing period during which said Compound 1 or a pharmaceutically acceptable salt thereof is administered, and (b) a resting period during which said Compound 1 or a pharmaceutically acceptable salt thereof is not administered.

In some embodiments, provided herein is the use of Compound 1 or a pharmaceutically acceptable salt thereof, in the manufacture of a medicament for the treatment of cancer, wherein the medicament is to be used in combination with a PD-1 inhibitor according to an intermittent dosing schedule of at least one dosing cycle, wherein each dosing cycle comprises (a) a dosing period during which said Compound 1 or a pharmaceutically acceptable salt thereof and said PD-1 inhibitor are administered, and (b) a resting period during which said Compound 1 or a pharmaceutically acceptable salt thereof and said PD-1 inhibitor are not administered.

In some embodiments, provided herein is the use of Compound 1 or a pharmaceutically acceptable salt thereof, in the manufacture of a medicament for the treatment of cancer, wherein the medicament is to be used in combination with a PD-L1 inhibitor according to an intermittent dosing schedule of at least one dosing cycle, wherein each dosing cycle comprises (a) a dosing period during which said Compound 1 or a pharmaceutically acceptable salt thereof and said PD-L1 inhibitor are administered, and (b) a resting period during which said Compound 1 or a pharmaceutically acceptable salt thereof and said PD-L1 inhibitor are not administered.

In some embodiments, provided herein is a kit comprising Compound 1 or a pharmaceutically acceptable salt thereof for use in the treatment of cancer, wherein Compound 1 or a pharmaceutically acceptable salt thereof is administered as a monotherapy according to an intermittent dosing schedule of at least one dosing cycle, wherein each dosing cycle comprises (a) a dosing period during which said Compound 1 or a pharmaceutically acceptable salt thereof is administered, and (b) a resting period during which said Compound 1 or a pharmaceutically acceptable salt thereof is not administered.

In some embodiments, provided herein is a kit comprising Compound 1 or a pharmaceutically acceptable salt thereof for use in the treatment of cancer, wherein Compound 1 or a pharmaceutically acceptable salt thereof is to be used in combination with a PD-1 inhibitor according to an intermittent dosing schedule of at least one dosing cycle, wherein each dosing cycle comprises (a) a dosing period during which said Compound 1 or a pharmaceutically acceptable salt thereof and said PD-1 inhibitor are administered, and (b) a resting period during which said Compound 1 or a pharmaceutically acceptable salt thereof and said PD-1 inhibitor are not administered.

In some embodiments, provided herein is a kit comprising Compound 1 or a pharmaceutically acceptable salt thereof for use in the treatment of cancer, wherein Compound 1 or a pharmaceutically acceptable salt thereof is to be used in combination with a PD-L1 inhibitor according to an intermittent dosing schedule of at least one dosing cycle, wherein each dosing cycle comprises (a) a dosing period during which said Compound 1 or a pharmaceutically acceptable salt thereof and said PD-L1 inhibitor are administered, and (b) a resting period during which said Compound 1 or a pharmaceutically acceptable salt thereof and said PD-L1 inhibitor are not administered.

In some embodiments, the subject was previously treated with at least one anticancer agent or therapy prior to said period of time (i.e., prior to said first dosing cycle). Examples of said at least one previous anticancer agent or therapy include a kinase inhibitor other than Compound 1 or a pharmaceutically acceptable salt thereof, an immunotherapy, chemotherapy, radiation therapy and/or surgery. In some embodiments of any of the methods described herein, the at least one anticancer agent or therapy that were administered to the patient before the period of time was unsuccessful (e.g., therapeutically unsuccessful as determined by a physician). In some embodiments the patient has a cancer that is resistant to the at least one anticancer agent.

In some embodiments of any of the methods described herein, the at least one anticancer agent is selected from the group of: a chemotherapeutic agent, a PI-3 kinase inhibitor, an EGFR inhibitor, a HER2/neu inhibitor, an FGFR inhibitor, an ALK inhibitor, an IGF1R inhibitor, a VEGFR inhibitor, a PDGFR inhibitor, a glucocorticoid, a BRAF inhibitor, a MEK inhibitor, a HER4 inhibitor, a MET inhibitor (e.g., a type I c-Met kinase inhibitor), a RAF inhibitor, an Akt inhibitor, a FTL-3 inhibitor, a MAP kinase pathway inhibitor.

In some embodiments of any of the methods described herein, the at least one anticancer agent can include a kinase inhibitor, and the patient previously developed resistance to the kinase inhibitor. In some embodiments of any of the methods described herein, the at least one anticancer agent includes a kinase inhibitor selected from the group of: crizotinib, capmatinib, NVP-BVU972, AMG 337, bozitinib, glumetinib, savolitinib, tepotinib, foretinib, bozitinib, 1-(6,7-dihydro-5H-benzo[6,7]cyclohepta[1,2-c]pyridazin-3-yl)-N3-[7(S)-(1-pyrrolidinyl)-6,7,8,9-tetrahydro-5H-benzocycloheptene-2-yl]-1H-1,2,4-triazole-3,5-diamine (BGB324), (N-[4-(2-Amino-3-chloropyridin-4-yloxy)-3-fluorophenyl]-4-ethoxy-1-(4-fluorophenyl)-2-oxo-1,2-dihydropyridine-3-carboxamide (BMS-777607), amuvatinib, BMS-796302, cabozantinib, glesatinib (MGCD265), 2-(4-fluorophenyl)-N-[3-fluoro-4-(3-phenyl-1H-pyrrolo[2,3-b]pyridin-4-yloxy)phenyl]-1,5-dimethyl-3-oxo-2,3-dihydro-1H-pyrazole-4-carboxamide (NPS-1034), N-[4-[(6,7-dimethoxyquinolin-4-yl)oxy]-3-fluorophenyl]-4-ethoxy-1-(4-fluoro-2-methylphenyl)-1H-pyrazole-3-carboxamide hydrochloride (LDC1267), gilteritinib, [3-(2-[[3-fluoro-4-(4-methylpiperazin-1-yl)phenyl]amino]-5-methyl-7H-pyrrolo[2,3-d]pyrimidin-4-yl)phenyl]acetonitrile (SGI-7079), dubermatinib (TP-0903), trans-4-[2-(butylamino)-5-[4-[(4-methylpiperazin-1-yl)methyl]phenyl]-7H-pyrrolo[2,3-d]pyrimidin-7-yl]cyclohexanol (UNC2025), 3-[3-[4-(Morpholin-4-ylmethyl)-1H-pyrrol-2-ylmethylene]-2-oxo-2,3-dihydro-1H-indol-5-ylmethyl]thiazolidine-2,4-dione hydrochloride (S49076), sunitinib, 12A11, Mab173, YW327.6S2, D9, E8, merestinib, [3-(2-[[3-fluoro-4-(4-methylpiperazin-1-yl)phenyl]amino]-5-methyl-7H-pyrrolo[2,3-d]pyrimidin-4-yl)phenyl]acetonitrile (SGI-7079), and N-[4-[(6,7-dimethoxyquinolin-4-yl)oxy]-3-fluorophenyl]-4-ethoxy-1-(4-fluoro-2-methylphenyl)-1H-pyrazole-3-carboxamide hydrochloride. In one embodiment of any of the methods disclosed herein, the patient was administered said kinase inhibitor for the treatment of cancer and the cancer became resistant to said at least one anticancer agent.

In some embodiments of any of the methods described herein, the at least one additional anticancer agent includes dexamethasone, and the patient previously developed resistance to dexamethasone. In some embodiments of any of the methods described herein, the at least one additional anticancer agent includes cytarabine, and the patient previously developed resistance to cytarabine. In some embodiments of any of the methods described herein, the at least one additional anticancer agent includes imatinib, and the patient previously developed resistance to imatinib. In some embodiments of any of the methods described herein, the at least one additional anticancer agent includes lapatinib, and the patient previously developed resistance to lapatinib. In some embodiments of any of the methods described herein, the at least one additional anticancer agent includes cetuximab, and the patient previously developed resistance to cetuximab. In some embodiments of any of the methods described herein, the at least one additional anticancer agent includes erlotinib, and the patient previously developed resistance to erlotinib. In some embodiments of any of the methods described herein, the at least one additional anticancer agent includes alpelisib, and the patient previously developed resistance to alpelisib. In some embodiments of any of the methods described herein, the at least one additional anticancer agent includes cisplatin, and the patient previously developed resistance to cisplatin. In some embodiments of any of the methods described herein, the at least one additional anticancer agent includes sunitinib, and the patient previously developed resistance to sunitinib. In some embodiments of any of the methods described herein, the at least one additional anticancer agent includes metformin, and the patient previously developed resistance to metformin.

In some embodiments of any of the methods described herein, the at least one additional anticancer agent includes an anti-PD1 antibody, and the patient previously developed resistance to the anti-PD1 antibody. In some embodiments of any of the methods described herein, the at least one additional anticancer agent includes docetaxel, and the patient previously developed resistance to docetaxel. In some embodiments of any of the methods described herein, the at least one additional anticancer agent includes an EGFR inhibitor, and the patient previously developed resistance to the EGFR inhibitor.

In one embodiment of any of the methods disclosed herein, the one or more therapeutic agents that are administered to the patient before the period of time is or includes chemotherapy. In one embodiment, the one or more therapeutic agents that are administered to the patient before the period of time is or includes a platinum-based chemotherapy. In one embodiment, the one or more therapeutic agents that are administered to the patient before the period of time is or includes a fluoropyrimidine-containing chemotherapy. In one embodiment, the one or more therapeutic agents that are administered to the patient before the period of time is or includes FOLFIRINOX (a chemotherapy regimen of folinic acid (leucovorin), fluorouracil (5-FU), irinotecan, and oxaliplatin). In one embodiment, the one or more therapeutic agents that are administered to the patient before the period of time is or includes FOLFOXIRI (a chemotherapy regimen of irinotecan and oxaliplatin plus 5-fluorouracil). In one embodiment, the cancer has progressed after treatment with a platinum-based chemotherapy.

In one embodiment, the patient has been administered surgery before the period of time. Non-limiting examples of surgery include, e.g., open surgery or minimally invasive surgery. Surgery can include, e.g., removing an entire tumor, debulking of a tumor, or removing a tumor that is causing pain or pressure in the subject. Methods for performing open surgery and minimally invasive surgery on a subject having a cancer are known in the art.

In one embodiment, the patient has received radiotherapy before the period of time. Non-limiting examples of radiation therapy include external radiation beam therapy (e.g., external beam therapy using kilovoltage X-rays or megavoltage X-rays) or internal radiation therapy. Internal radiation therapy (also called brachytherapy) can include the use of, e.g., low-dose internal radiation therapy or high-dose internal radiation therapy. Low-dose internal radiation therapy includes, e.g., inserting small radioactive pellets (also called seeds) into or proximal to a cancer tissue in the subject. High-dose internal radiation therapy includes, e.g., inserting a thin tube (e.g., a catheter) or an implant into or proximal to a cancer tissue in the subject, and delivering a high dose of radiation to the thin tube or implant using a radiation machine. Methods for performing radiation therapy on a subject having a cancer are known in the art.

Compound 1 or a pharmaceutically acceptable salt thereof may be formulated prior to administration. The formulation will preferably be adapted to the particular mode of administration. Compound 1 or a pharmaceutically acceptable salt thereof may be formulated with pharmaceutically acceptable carriers as known in the art and administered in a wide variety of dosage forms as known in the art. In making the pharmaceutical compositions of the present invention, Compound 1 or a pharmaceutically acceptable salt thereof will usually be mixed with a pharmaceutically acceptable carrier or diluted by a carrier or enclosed within a carrier. Such carriers include, but are not limited to, solid diluents or fillers, excipients, sterile aqueous media and various non-toxic organic solvents. Dosage unit forms or pharmaceutical compositions include tablets, capsules, such as gelatin capsules, pills, powders, granules, aqueous and nonaqueous oral solutions and suspensions, lozenges, troches, hard candies, sprays, creams, salves, suppositories, jellies, gels, pastes, lotions, ointments, injectable solutions, elixirs, syrups, and parenteral solutions packaged in containers adapted for subdivision into individual doses.

In one embodiment of any of said methods disclosed herein, Compound 1 is administered as the free base. In one embodiment of any of said methods disclosed herein, Compound 1 is administered as Compound 1 HCl. In one embodiment, Compound 1 or a pharmaceutically acceptable salt thereof is administered as a capsule. In one embodiment, a capsule formulation of Compound 1 comprises about 5 mg to about 50 mg (e.g., 5 mg to about 45 mg, about 5 mg to about 40 mg, about 5 mg to about 35 mg, about 5 mg to about 30 mg, about 5 mg to about 25 mg, about 5 mg to about 20 mg, about 5 mg to about 18 mg, about 5 mg to about 16 mg, about 5 mg to about 14 mg, about 5 mg to about 12 mg, about 5 mg to about 10 mg, about 5 mg to about 8 mg, about 10 mg to about 50 mg, about 10 mg to about 45 mg, about 10 mg to about 40 mg, about 10 mg to about 35 mg, about 10 mg to about 30 mg, about 10 mg to about 25 mg, about 10 mg to about 20 mg, about 10 mg to about 18 mg, about 10 mg to about 16 mg, about 10 mg to about 14 mg, about 10 mg to about 12 mg, about 12 mg to about 50 mg, about 12 mg to about 45 mg, about 12 mg to about 45 mg, about 12 mg to about 40 mg, about 12 mg to about 35 mg, about 12 mg to about 30 mg, about 12 mg to about 25 mg, about 12 mg to about 20 mg, about 12 mg to about 18 mg, about 12 mg to about 16 mg, about 12 mg to about 14 mg, about 14 mg to about 50 mg, about 14 mg to about 45 mg, about 14 mg to about 40 mg, about 14 mg to about 35 mg, about 14 mg to about 30 mg, about 14 mg to about 25 mg, about 14 mg to about 20 mg, about 14 mg to about 18 mg, about 14 mg to about 16 mg, about 16 mg to about 50 mg, about 16 mg to about 45 mg, about 16 mg to about 40 mg, about 16 mg to about 35 mg, about 16 mg to about 30 mg, about 16 mg to about 25 mg, about 16 mg to about 20 mg, about 16 mg to about 18 mg, about 18 mg to about 50 mg, about 18 mg to about 45 mg, about 18 mg to about 40 mg, about 18 mg to about 35 mg, about 18 mg to about 30 mg, about 18 mg to about 25 mg, about 18 mg to about 20 mg, about 20 mg to about 50 mg, about 20 mg to about 45 mg, about 20 mg to about 40 mg, about 20 mg to about 35 mg, about 20 mg to about 30 mg, about 20 mg to about 25 mg, about 25 mg to about 50 mg, about 25 mg to about 45 mg, about 25 mg to about 40 mg, about 25 mg to about 35 mg, about 25 mg to about 30 mg, about 30 mg to about 50 mg, about 30 mg to about 45 mg, about 30 mg to about 40 mg, about 30 mg to about 35 mg, about 35 mg to about 50 mg, about 35 mg to about 45 mg, about 35 mg to about 40 mg, about 40 mg to about 50 mg, about 40 mg to about 45 mg, about 45 mg to about 50 mg, about 5 mg, about 10 mg, about 15 mg, about 20 mg, about 25 mg, about 30 mg, about 35 mg, about 40 mg, about 45 mg, or about 50 mg) of Compound 1 or a pharmaceutically acceptable salt thereof.

Those skilled in the art will be able to determine, according to known methods, the appropriate amount, dose or dosage of each compound, as used in the combination of the present invention, to administer to a patient, taking into account factors such as age, weight, general health, the compound administered, the route of administration, the nature and advancement of the cancer requiring treatment, and the presence of other medications.

Treatment according to any of the methods disclosed herein may be assessed with one or more clinical endpoints, for example by inhibition of disease progression, inhibition of tumor growth, reduction of primary tumor, relief of tumor-related symptoms, inhibition of tumor secreted factors (including expression levels of checkpoint proteins as identified herein), delayed appearance of primary or secondary tumors, slowed development of primary or secondary tumors, decreased occurrence of primary or secondary tumors, slowed or decreased severity of secondary effects of disease, arrested tumor growth and regression of tumors, increased Time To Progression (TTP), improved Time to tumor response (TTR), increased duration of response (DR), increased Progression Free Survival (PFS), increased Overall Survival (OS), Objective Response Rate (ORR), among others. OS as used herein means the time from treatment onset until death from any cause. As used herein, TTP means the time from treatment onset until tumor progression; TTP does not comprise deaths. As used herein, TTR is defined for patients with confirmed objective response (CR or PR) as the time from the date of randomization or date of first dose of study treatment to the first documentation of objective tumor response. As used herein, DR means the time from documentation of tumor response to disease progression. As used herein, PFS means the time from treatment onset until tumor progression or death. As used herein, ORR means the proportion of patients with tumor size reduction of a predefined amount and for a minimum time period, where response duration usually is measured from the time of initial response until documented tumor progression. In the extreme, complete inhibition, is referred to herein as prevention or chemoprevention.

In one embodiment, a subject treated according to any of the methods disclosed herein may be assessed according to one or more clinical endpoints associated with treating a cancer with a combination therapy described herein. In one embodiment, a patient described herein can show a positive tumor response, such as inhibition of tumor growth or a reduction in tumor size after treatment with a combination described herein. In certain embodiments, a patient described herein can achieve a Response Evaluation Criteria in Solid Tumors (for example, RECIST 1.1) of complete response, partial response or stable disease after administration of an effective amount a combination therapy described herein. In certain embodiments, a patient described herein can show increased survival without tumor progression. In some embodiments, a patient described herein can show inhibition of disease progression, inhibition of tumor growth, reduction of primary tumor, relief of tumor-related symptoms, inhibition of tumor secreted factors (including tumor secreted hormones, such as those that contribute to carcinoid syndrome), delayed appearance of primary or secondary tumors, slowed development of primary or secondary tumors, decreased occurrence of primary or secondary tumors, slowed or decreased severity of secondary effects of disease, arrested tumor growth and regression of tumors, decreased Time to Tumor Response (TTR), increased Duration of Response (DR), increased Progression Free Survival (PFS), increased Time To Progression (TTP), and/or increased Overall Survival (OS), among others.

It may be shown by established test models that a therapy described herein results in the beneficial effects described herein before. The person skilled in the art is fully enabled to select a relevant test model to prove such beneficial effects. The pharmacological activity of a combination therapy described herein may, for example, be demonstrated in an animal model and/or a clinical study or in a test procedure, for example as described below.

Suitable clinical studies are, for example, open label, dose escalation studies in patients with a proliferative disease. The beneficial effects on proliferative diseases may be determined directly through the results of these studies. Such studies may, in particular, be suitable for comparing the effects of a monotherapy using Compound 1 or a pharmaceutically acceptable salt thereof and/or the PD-1 inhibitor versus the effects of a combination therapy comprising Compound 1 or a pharmaceutically acceptable salt thereof and the PD-1 inhibitor. The efficacy of the treatment may be determined in such studies, e.g., after 6, 12, 18 or 24 weeks by evaluation of symptom scores, e.g., every 6 weeks.

Also provided herein are Embodiments 1-34.

Embodiment 1. A solid dispersion comprising amorphous (R)—N-(3-fluoro-4-((3-((1-hydroxypropan-2-yl)amino)-1H-pyrazolo[3,4-b]pyridin-4-yl)oxy)phenyl)-3-(4-fluorophenyl)-1-isopropyl-2,4-dioxo-1,2,3,4-tetrahydropyrimidine-5-carboxamide hydrochloride dispersed in a polymer.

Embodiment 2. The solid dispersion of Embodiment 1, wherein the polymer is a cellulose-based polymer.

Embodiment 3. The solid dispersion of Embodiment 2, wherein the cellulose-based polymer is selected from a methylcellulose, a hydroxypropyl methylcellulose, a hypromellose phthalate, a hydroxypropyl methylcellulose acetate succinate, and co-polymers thereof.

Embodiment 4. The solid dispersion of Embodiment 3, wherein the cellulose-based polymer is a hydroxypropyl methylcellulose acetate succinate (HPMCAS).

Embodiment 5. The solid dispersion of Embodiment 4, wherein the hydroxypropyl methylcellulose acetate succinate is hydroxypropyl methylcellulose acetate succinate-MG.

Embodiment 6. The solid dispersion according to any one of Embodiments 2-5, wherein (R)—N-(3-fluoro-4-((3-((1-hydroxypropan-2-yl)amino)-1H-pyrazolo[3,4-b]pyridin-4-yl)oxy)phenyl)-3-(4-fluorophenyl)-1-isopropyl-2,4-dioxo-1,2,3,4-tetrahydropyrimidine-5-carboxamide hydrochloride is present in a concentration range of 25% to 50% w/w based on total weight of the solid dispersion, calculated as the free base of (R)—N-(3-fluoro-4-((3-((1-hydroxypropan-2-yl)amino)-1H-pyrazolo[3,4-b]pyridin-4-yl)oxy)phenyl)-3-(4-fluorophenyl) isopropyl-2,4-dioxo-1,2,3,4-tetrahydropyrimidine-5-carboxamide.

Embodiment 7. The solid dispersion according to Embodiment 6, wherein (R)—N-(3-fluoro-4-((3-((1-hydroxypropan-2-yl)amino)-1H-pyrazolo[3,4-b]pyridin-4-yl)oxy)phenyl)-3-(4-fluorophenyl)-1-isopropyl-2,4-dioxo-1,2,3,4-tetrahydropyrimidine-5-carboxamide is present in an amount of about 25%, 37.5% or 50% w/w.

Embodiment 8. The solid dispersion according to Embodiment 6, wherein (R)—N-(3-fluoro-4-((3-((1-hydroxypropan-2-yl)amino)-1H-pyrazolo[3,4-b]pyridin-4-yl)oxy)phenyl)-3-(4-fluorophenyl)-1-isopropyl-2,4-dioxo-1,2,3,4-tetrahydropyrimidine-5-carboxamide is present in an amount of about 25% w/w.

Embodiment 9. The solid dispersion according to any one of Embodiments 2-8, wherein the solid dispersion is a spray-dried dispersion.

Embodiment 10. Use of a solid dispersion according to any one of Embodiments 2-8 in the preparation of a pharmaceutical composition.

Embodiment 11. A pharmaceutical composition comprising (i) a solid dispersion according to any one of Embodiments 2-9, (ii) one or more intragranular excipients, and (iii) one or more extragranular excipients.

Embodiment 12. A pharmaceutical composition comprising (i) a solid dispersion according to any one of Embodiments 2-9, (ii) one or more intragranular excipients, wherein at least one of said intragranular excipients is a pH modifying agent, and (iii) one or more extragranular excipients.

Embodiment 13. The pharmaceutical composition comprising a solid dispersion according to Embodiment 11, wherein the one or more intragranular excipients are a pH modifying agent, a filler, a disintegrant, and a lubricant.

Embodiment 14. The pharmaceutical composition comprising a solid dispersion according to Embodiment 13, wherein the pH modifying agent is sodium bicarbonate.

Embodiment 15. The pharmaceutical composition comprising a solid dispersion according to Embodiment 13 or 14, wherein the filler is mannitol.

Embodiment 16. The pharmaceutical composition comprising a solid dispersion according to any one of Embodiments 13 to 15, wherein the disintegrant is croscarmellose sodium.

Embodiment 17. The pharmaceutical composition comprising a solid dispersion according to any one of Embodiments 13 to 16, wherein the lubricant is stearyl fumarate sodium.

Embodiment 18. The pharmaceutical composition comprising a solid dispersion according to any one of Embodiments 13-17, wherein the one or more extragranular excipients are a filler and a disintegrant.

Embodiment 19. The pharmaceutical composition comprising a solid dispersion according to Embodiment 18, wherein the filler is mannitol.

Embodiment 20. The pharmaceutical composition comprising a solid dispersion according to Embodiment 18 or 19, wherein the disintegrant is croscarmellose sodium.

Embodiment 21. The pharmaceutical composition comprising a solid dispersion according to Embodiment 11, comprising: (i) a solid dispersion of amorphous Compound 1 HCl in HPMCAS-MG, (ii) intragranular excipients comprising a pH modifying agent, a filler, a disintegrant and a lubricant, and (iii) extragranular excipients comprising a filler and a disintegrant.

Embodiment 22. The pharmaceutical composition comprising a solid dispersion according to Embodiment 21, comprising (i) a solid dispersion of amorphous Compound 1 HCl in HPMCAS-MG, wherein said solid dispersion is in an amount between about 10% and 43% w/w, (ii) intragranular excipients comprising a pH modifying agent in an amount between 1% and 4% w/w, a filler in an amount between about 6% and 25% w/w, a disintegrant in an amount between about 1.0% and 4% w/w, a lubricant in an amount between about 0.5% and 2% w/w, and (iii) extragranular excipients comprising a filler in an amount between about 20% and 75% w/w and a disintegrant in an amount between about 2% and 8% w/w.

Embodiment 23. The pharmaceutical composition comprising a solid dispersion according to Embodiment 21, comprising (i) a solid dispersion of amorphous Compound 1 HCl in HPMCAS-MG, wherein said solid dispersion is in an amount between about 10% and 43% w/w, (ii) intragranular excipients comprising a pH modifying agent which is sodium bicarbonate in an amount between 1% and 4% w/w, a filler which is mannitol in an amount between about 6% and 25% w/w, a disintegrant which is croscarmellose sodium in an amount between about 1.0% and 4% w/w, and a lubricant which is sodium stearyl fumarate in an amount between about 0.5% and 2% w/w and (iii) extragranular excipients comprising a filler which is mannitol in an amount between about 20% and 75% w/w and a disintegrant which is croscarmellose sodium in an amount between about 2% and 8% w/w.

Embodiment 24. The pharmaceutical composition comprising a solid dispersion according to Embodiment 21, comprising (i) 11.1% w/w of a solid dispersion of amorphous Compound 1 HCl in HPMCAS-MG, (ii) intragranular excipients comprising 1% w/w of a pH modifying agent which is sodium bicarbonate, 6.6% w/w of a filler which is mannitol, 1% w/w of a disintegrant which is croscarmellose sodium, and 0.5% w/w of a lubricant which is sodium stearyl fumarate, and (iii) extragranular excipients comprising 72.2% of a filler which is mannitol and 7.6% w/w of a disintegrant which is croscarmellose sodium.

Embodiment 25. The pharmaceutical composition according to Embodiment 24, comprising 5 mg of Compound 1 calculated as the free base.

Embodiment 26. The pharmaceutical composition according to Embodiment 24 or 25, wherein the solid dispersion comprises 25%:75% w/w amorphous Compound 1 HCl:HPMCAS-MG.

Embodiment 27. The pharmaceutical composition comprising a solid dispersion according to Embodiment 21, comprising (i) 28.5% w/w of a solid dispersion of amorphous Compound 1 HCl in HPMCAS-MG, (ii) intragranular excipients comprising 2.6% w/w of a pH modifying agent which is sodium bicarbonate, 16.9% w/w of a filler which is mannitol, 2.6% w/w of a disintegrant which is croscarmellose sodium, and 1.3% w/w of a lubricant which is sodium stearyl fumarate, and (iii) extragranular excipients comprising 43.5% of a filler which is mannitol and 4.6% w/w of a disintegrant which is croscarmellose sodium.

Embodiment 28. The pharmaceutical composition according to Embodiment 27, comprising 25 mg of Compound 1 calculated as the free base.

Embodiment 29. The pharmaceutical composition according to Embodiment 27 or 28, wherein the solid dispersion comprises 25%:75% w/w amorphous Compound 1 HCl:HPMCAS-MG.

Embodiment 30. The pharmaceutical composition comprising a solid dispersion according to Embodiment 21, comprising (i) 42.1% w/w of a solid dispersion of amorphous Compound 1 HCl in HPMCAS-MG, (ii) intragranular excipients comprising 3.8% w/w of a pH modifying agent which is sodium bicarbonate, 24.9% w/w of a filler which is mannitol, 3.8% w/w of a disintegrant which is croscarmellose sodium, and 1.9% w/w of a lubricant which is sodium stearyl fumarate, and (iii) extragranular excipients comprising 21.3% of a filler which is mannitol and 2.2% w/w of a disintegrant which is croscarmellose sodium.

Embodiment 31. The pharmaceutical composition according to Embodiment 30, comprising 50 mg of Compound 1 calculated as the free base.

Embodiment 32. The pharmaceutical composition according to Embodiment 30 or 31, wherein the solid dispersion comprises 25%:75% w/w amorphous Compound 1 HCl:HPMCAS-MG.

Embodiment 33. The pharmaceutical composition comprising a solid dispersion according to any one of Embodiments 11-32, wherein the composition is in the form of a capsule.

Embodiment 34. A method of treating cancer in a patient in need thereof, comprising administering a therapeutically effective amount of a pharmaceutical composition according to any one of Embodiments 11-33.

Unless otherwise defined, all technical and scientific terms used herein have the same meaning as commonly understood by one of ordinary skill in the art to which this invention belongs. In case of conflict, the present specification, including definitions, will control. Unless otherwise required by context, singular terms shall include pluralities and plural terms shall include the singular. As used herein, the singular form “a”, “an”, and “the” include plural references unless indicated otherwise. For example, “an” excipient includes one or more excipients.

Exemplary methods and materials are described herein, although methods and materials similar or equivalent to those described herein can also be used in the practice or testing of the invention. The materials, methods, and examples are illustrative only and not intended to be limiting.

### EXAMPLES

### Synthetic Examples

### Example A

### Preparation of amorphous (R)—N-(3-fluoro-4-((3-((1-hydroxypropan-2-yl)amino)-1H-pyrazolo[3,4-b]pyridin-4-yl)oxy)phenyl)-3-(4-fluorophenyl)-1-isopropyl-2,4-dioxo-1,2,3,4-tetrahydropyrimidine-5-carboxamide hydrochloride

(R)—N-(3-fluoro-4-((3-((1-hydroxypropan-2-yl)amino)-1H-pyrazolo[3,4-b]pyridin-4-yl)oxy)phenyl)-3-(4-fluorophenyl)-1-isopropyl-2,4-dioxo-1,2,3,4-tetrahydropyrimidine-5-carboxamide (14.4 g, 24.3 mmol) was dissolved in 10% MeOH in dichloromethane (200 mL) and hydrogen chloride (4.87 mL, 24.3 mmol) (5-6 M in isopropyl alcohol) was added. The mixture was stirred for 10 min and then added slowly to Et₂O (1.2 L) with stirring. The mixture was stirred for 20 min, and the resulting solids were isolated by filtration and dried on high vacuum overnight to afford amorphous (R)—N-(3-fluoro-4-((3-((1-hydroxypropan-2-yl)amino)-1H-pyrazolo[3,4-b]pyridin-4-yl)oxy)phenyl)-3-(4-fluorophenyl)-1-isopropyl-2,4-dioxo-1,2,3,4-tetrahydropyrimidine-5-carboxamide hydrochloride (11.9 g, 18.9 mmol, 77.8% yield) as a yellow solid.

### Example B

### Spray-Dried Dispersions

Spray-dried dispersions of amorphous (R)—N-(3-fluoro-4-((3-((1-hydroxypropan yl)amino)-1H-pyrazolo[3,4-b]pyridin-4-yl)oxy)phenyl)-3-(4-fluorophenyl)-1-isopropyl-2,4-dioxo-1,2,3,4-tetrahydropyrimidine-5-carboxamide hydrochloride were prepared using the following procedure.

TABLE X
Spray-dried dispersion formulations of Compound 1 HCI

| | | Compound 1 HCI |
|---|---|---|
| Sample | Ingredients | content (w/w %) |
| 1 | Amorphous Compound 1 | 25 |
| | HCI in HPMCAS-M | |
| 2 | Amorphous Compound 1 | 37.5 |
| | HCI in HPMCAS-M | |
| 3 | Amorphous Compound 1 | 50 |
| | HCI in HPMCAS-M | |

The spray solutions were prepared by adding dichloromethane and methanol (60:40 dichloromethane:methanol) to a stainless steel mixing vessel. The polymer was added to the solvent system while mixing with a top down mixer at a medium vortex until the polymer is completely solubilized. Amorphous Compound 1 HCl was then added to the solution at medium vortex and left to mix until completely solubilized. The spray solution was passed through an inline filter during spraying. After drying, the spray dried dispersion was dried in an oven. Spray dried dispersion particles were collapsed spheres of 5-20 microns.

Table Y provides details for the preparation of a spray dried dispersion of amorphous Compound 1 HCl according to one embodiment.

TABLE Y
SDD Manufacturing Summary

| SDD Formulation | amorphous Compound 1 HCI:HPMCAS-M |
|---|---|
| Batch Size (g API/g Total Solids) | 851 g Drug Substance/3252 g Total Solids |
| Yield (%) | 92.4% |

Spray Drying Process Summary

| | |
|---|---|
| Spray Solvent | 60% (w/w) DCM, 40% (w/w) MeOH |
| Total Solid Content | 10% (w/w) |
| Drug substance content | 2.65% (w/w) |
| (90.9% free base, 5.6% | |
| HCI, and 3.5% impurities) | |
| Polymer content | 7.35% (w/w) |
| Duration (Total Time in Solution) | 70 hours |

Spray Dryer Parameter Summary

| Spray Dryer | Procept 4M8TRX | | |
|---|---|---|---|
| Nozzle Configuration | 0.6 mm tip/0.6 mm cap/no cone on cap | | |
| Atomization Pressure | 3.00 bar | | |
| Solution Flow Rate | Average: 121 rpm | Min: 116 rpm | Max: 138 rpm |
| | 0.91 L/min (approx) | | |
| Drying Gas Flow Rate | 0.70 mm³/min | | |
| Tᵢₙ (° C.) | Average: 74.9 | Min: 74.0 | Max: 75.7 |
| Tₒᵤₜ (° C.) | Average: 47.2 | Min: 44.2 | Max: 49.2 |

Tray Drying Summary

| | |
|---|---|
| Temperature (° C.) | 40° C. |
| Time | 24 hr |

SDD Characterization Summary

| Use Potency * | 22.9% | |
|---|---|---|
| Purity | 98.8% | |
| Residual Solvent | DCM: 39.7 ppm | MeOH: 57.5 ppm |

| | |
|---|---|
| Tg | 124° C. |
| Non Sink Dissolution Profile | |
| Solubility Enhancement | 3.90 |
| (relative to API) | |
| AUC - 360 min Enhancement | 1.58 |
| (relative to API) | |

* Use potency accounts for chiral purity and is calculated based on a standard concentration that has been potency corrected based on q-NMR potency.

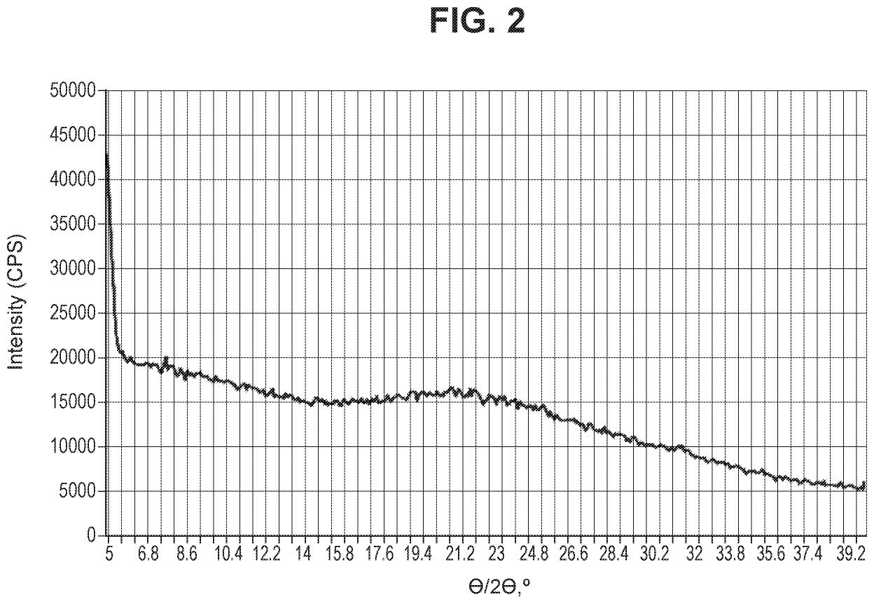

XRPD and SEM analyses were conducted on Samples 1, 2 and 3 and data from both analyses for each sample confirmed that spray dried dispersion is amorphous and free of any detectable levels of crystalline material after three months. An XRPD scan of a spray-dried dispersion of Sample 1 is shown in FIG. 2.

### Example C

### Capsule Formulation

Capsules comprising a spray-dried dispersion of amorphous Compound 1 HCl in HPMCAS-M were prepared using the following procedure.

### Composition of Compound 1 HCl Spray-Dried Dispersion Capsules, 5 mg

| | | | |
|---|---|---|---|
| | | Amount per | Content |
| Component | Function | capsule (mg) | (% w/w) |

Intragranular

| | | | |
|---|---|---|---|
| Amorphous Compound | Active | 20.0 | 11.1 |
| 1 HCI spray dried | ingredient | | |
| dispersion | | | |
| Mannitol | Filler | 11.8 | 6.6 |
| Croscarmellose sodium | Disintegrant | 1.8 | 1.0 |
| Sodium stearyl fumarate | Lubricant | 0.9 | 0.5 |
| Sodium bicarbonate | pH modifying agent | 1.8 | 1.0 |

Extragranular

| | | | |
|---|---|---|---|
| Mannitol | Filler | 130.0 | 72.2 |
| Croscarmellose sodium | disintegrant | 13.6 | 7.6 |

| | | |
|---|---|---|
| Total Target Fill Weight | 180 mg | 100.00 |

HMPC Capsule Shell

| Capsule shell (Swedish | Encapsulation | 1 × Size “3” capsule | |
|---|---|---|---|
| Orange HMPC capsules) | | | |
| Hypromellose (HMPC) | Structure | 45.72 | qs 100 |
| Titanium dioxide (E171) | Opacifier | 0.23 | 0.4916 |
| Red iron oxide (E172) | Colorant | 0.55 | 1.187 |

| | | |
|---|---|---|
| Approximate weight of | 46.50 | N/A |
| capsule shell | | |

### Composition of Compound 1 HCl Spray-Dried Dispersion Capsules, 25 mg

| | | | |
|---|---|---|---|
| | | Amount per | Content |
| Component | Function | capsule (mg) | (% w/w) |

Intragranular

| | | | |
|---|---|---|---|
| Amorphous Compound | Active | 99.9 | 28.5 |
| 1 HCI spray dried | ingredient | | |
| dispersion | | | |
| Mannitol | Filler | 59.0 | 16.9 |
| Croscarmellose sodium | Disintegrant | 9.1 | 2.6 |
| Sodium stearyl fumarate | Lubricant | 4.5 | 1.3 |
| Sodium bicarbonate | pH modifying agent | 9.1 | 2.6 |

Extragranular

| | | | |
|---|---|---|---|
| Mannitol | Filler | 152.4 | 43.5 |
| Croscarmellose sodium | disintegrant | 16.0 | 4.6 |

| | | |
|---|---|---|
| Total Target Fill Weight | 350.0 | 100.0 |

HMPC Capsule Shell

| Capsule shell (Swedish | Encapsulation | 1 × Size “1” capsule | |
|---|---|---|---|
| Orange HMPC capsules) | | | |
| Hypromellose (HMPC) | Structure | 74.73 | qs 100 |
| Titanium dioxide (E171) | Opacifier | 0.37 | 0.4916 |
| Red iron oxide (E172) | Colorant | 0.90 | 1.187 |

| | | |
|---|---|---|
| Approximate weight of | 76.00 | N/A |
| capsule shell | | |

### Composition of Compound 1 HCl Spray-Dried Dispersion Capsules, 50 mg

| | | | |
|---|---|---|---|
| | | Amount per | |
| | | capsule | Content |
| Component | Function | (mg) | (% w/w) |

Intragranular

| | | | |
|---|---|---|---|
| Amorphous Compound 1 HCl | Active | 199.9 | 42.1 |
| spray dried dispersion | ingredient | | |
| Mannitol | Filler | 118.1 | 24.9 |
| Croscarmellose sodium | Disintegrant | 18.2 | 3.8 |
| Sodium stearyl fumarate | Lubricant | 9.1 | 1.9 |
| Sodium bicarbonate | pH modifying | 18.2 | 3.8 |
| | agent | | |

Extragranular

| | | | |
|---|---|---|---|
| Mannitol | Filler | 101.0 | 21.3 |
| Croscarmellose sodium | disintegrant | 10.6 | 2.2 |

Total Target Fill Weight

HMPC Capsule Shell

| Capsule shell (Swedish | Encapsulation | 1 × Size “0” capsule | |
|---|---|---|---|
| Orange HMPC capsules) | | | |
| Hypromellose (HMPC) | Structure | 91.93 | qs 100 |
| Titanium dioxide (E171) | Opacifier | 0.46 | 0.4916 |
| Red iron oxide (E172) | Colorant | 1.11 | 1.187 |

| | | |
|---|---|---|
| Approximate weight of capsule shell | 93.50 | N/A |

The spray dried dispersion was prepared as described in Example B. The intragranular components were combined and blended, and then sieved through a screen. The sieved substance underwent dry granulation and milling. The extragranular components were combined with the granulated and milled components and the mixture was blended and then sieved through a screen. The sieved substance was then encapsulated.

### BIOLOGICAL EXAMPLES

### Example 1

### Exploratory Study Evaluating Dose-Scheduling, Activity, and Ocular Toxicity of Compound 1 in Syngeneic MC-38 Colon Adenocarcinoma Tumor-Bearing Mice

### Objectives and Study Design

This study was designed to determine the tolerability, efficacy, and ocular toxicity of Compound 1, administered as a monotherapy or in combination with anti-PD-1 with variable dosing schedules in an immune-competent model of colon adenocarcinoma.

### SUMMARY

Female C57BL/6 bearing syngeneic MC-38 colon adenocarcinoma tumors (n=8/group, implanted on Day 0) were administered test articles Compound 1 or anti-PD-1, as monotherapy or in combination, starting on Day 10. As monotherapy, anti-PD-1 was administered twice weekly (BIW) for 2 consecutive weeks by intraperitoneal (IP) injection at 200 μg/mouse (Days 10, 13, 17, and 20). As monotherapy, Compound 1 was administered twice daily (BID) at 50 mg/kg by oral gavage (PO) for 28 consecutive days (Days 10-37). As combination therapy with anti-PD-1 (administered at the dose and schedule indicated above), Compound 1 was administered BID at 50 mg/kg PO for 1) 7 consecutive days (Days 10-16); 2) 14 consecutive days (Days 10-23); 3) 21 consecutive days (Days 10-30); 4) 28 consecutive days (Days 10-37); or, 5) 7 consecutive days on the first and third week of dosing (Days 10-16 and Days 24-30). This set of cohorts was used to determine both the tolerability and efficacy of Compound 1 treatment across dosing schedules, as well as ocular toxicity upon recovery on Day 66.

Furthermore, tumor-naïve female C57BL/6 mice (n=5/group) were administered test articles Compound 1 or anti-PD-1, as monotherapy or in combination, starting on Day 10, with the identical doses and schedules as indicated above. This set of cohorts was used to determine the tolerability and ocular toxicity across dosing schedules of Compound 1 treatment on Day 38.

The doses and schedules employed in this study were well tolerated in all groups. Among the tumor-bearing cohorts, maximum body weight loss was <5.2%, with three early deaths possibly attributed to test article administration. Among the tumor-naïve cohorts, body weight loss was not observed, with two deaths possibly attributed to test article administration.

Tumor volume and body weight measurements were taken until Day 66 (56 days after the first dose for any cohort). Tumor growth inhibition (TGI) compared to the vehicle control arm was calculated on Day 32, the last day for which the vehicle control arm was intact. Anti-PD-1 monotherapy was minimally efficacious in this model, with two weeks of treatment resulting in 76.1% TGI, 45 days median survival (versus 31 days median survival for vehicle control), and 0/8 animals cured on Day 66 (versus 0/8 cures for vehicle control). Compound 1 as monotherapy dosed for 28 consecutive days was more efficacious, with twice daily treatment resulting in 95.6% TGI, 66 days median survival, and 0/6 animals cured. However, equivalent maximal efficacies were observed across all the combination therapy cohorts, particularly in cure rate. Compound 1 dosed for 7 days in combination with anti-PD-1 resulted in 96.6% TGI and 6/8 cures, while the interval dosing schedule (Compound 1 on Days 10-16 and Days 24-30) in combination with anti-PD-1 similarly resulted in 96.2% TGI and 4/8 cures. Compound 1 dosed for 14 days, 21 days, or 28 days in combination with anti-PD-1 resulted in 97.1%, 98.5%, and 99.3% TGI, respectively, and these three cohorts demonstrated cure rates of 3/8, 3/8, and 5/7, respectively. Finally, among these combination therapy cohorts, the median survival was not reached in any group except for the one dosed with Compound 1 for 14 days, which yielded a median survival of 66 days.

Eyes were harvested for histopathology assessment from the tumor-naïve groups as the terminal necropsy cohort on Day 38, the last day the 28-day dosing period. Since not all terminal necropsy groups were dosed for the entirety of the 28-day dosing period, some groups effectively experienced a 1-3 week recovery period. Eyes were harvested for histopathology assessment from the tumor-bearing groups as the recovery necropsy cohort on Day 66, the last day of the 28-day recovery period. Since not all recovery necropsy groups were dosed for the entirety of the 28-day dosing period, some of these groups effectively experienced an extended recovery period beyond the 28-day recovery period of an additional 1-3 weeks. At terminal necropsy, Compound 1-related microscopic changes were limited to multifocal swelling of the rod and cone processes in the retina of mice that were administered Compound 1 for 28 days (as monotherapy or in combination with anti-PD-1). This finding was only visualized under epifluorescent illumination and was characterized by scattered individual processes that were increased in diameter up to five times the surrounding processes, with increased autofluorescence. At recovery sacrifice, no definitively Compound 1-related changes were observed, indicating that the terminal necropsy findings from animals treated with Compound 1 for four weeks all resolved after the 28-day recovery period and suggesting that one week of recovery after three weeks of Compound 1 treatment is be sufficient to allow for clearance of any microscopic changes that may have accumulated. It remained unclear whether these findings were adverse, as no degenerative changes were observed. If the material affected vision, it would be considered adverse; however, as this study was not designed to test for alterations in vision, adversity could not be determined.

Overall, these data support the conclusion that Compound 1 is tolerated as monotherapy and in combination with anti-PD-1, provides tumor control as monotherapy in the MC-38 murine model of colon adenocarcinoma in an immune-competent background, and substantially adds to efficacy in combination with anti-PD-1. Furthermore, this study demonstrates equivalent tumor growth inhibition, cure rate, and survival across dose schedules from 7 days to 28 days of Compound 1 treatment in combination with anti-PD-1. Finally, histopathology assessment of the retinas indicates that Compound 1-related microscopic changes may be resolved within a 1-4 week recovery period.

### Methods

### Test Articles

Oral vehicle {0.5% Affinisol™, pH 5 [hydroxypropyl methylcellulose (HPMC) hot melt extrusion (HME) 100LV Hypromellose, Dow Chemical Company ID99015561, Lot No. INR477140, in water]}. To prepare, 50 mL sterile water for injection was added gradually to 250 mg Affinisol™ with continuous agitation to create a clear solution.
200 μg anti-murine PD-1 (anti-PD-1, Clone RMP1-14, Rat IgG2a, BioXCell cat. no. BP0146, lot no. 640517M1B)—6.61 mg/mL stock concentration. RMP1-14 was prepared by dilution of 6.05 mL stock with 13.95 mL sterile saline for injection (Bimeda-MTC Animal Health Inc., VET ONE™). The antibody was administered at 200 μg/animal (100 μL of a 2 mg/mL dose solution) by intraperitoneal injection.
50 mg/kg Compound 1 [1:3 Compound 1 HCl:hydroxypropylmethylcellulose acetate succinate (HPMCAS) spray-dried dispersion (SDD, prepared by Seran Bioscience, Bend, Oreg.) was prepared as a yellow suspension in 0.5% Affinisol™. To prepare, 234.6 mL 0.5% Affinisol™ was added gradually to 8500 mg of the SDD, followed by vortexing and sonication. Subsequently, 156.4 mL of 0.5% Affinisol™, 13.59 mM NaOH was added to adjust to pH 4 for a final volume of 391 mL of the dosing solution at 50 mg/kg. The final dose suspension was stored at 4° C. and shaken/sonicated to resuspend prior to dose administration.

### Experimental Animals

Female C57BL/6 mice from the Jackson Laboratory (Sacramento, Calif.) born on 16 Jul. 2019 were obtained at nine weeks of age and housed in groups of four to acclimate for five days.

For initial tumor outgrowth, a suspension of 1×10⁶ MC-38 cells in a volume of 100 μL PBS was implanted subcutaneously on the right flank of the animal near the axillary region. Animals were then randomly assigned to treatment groups. Treatment was initiated on day 10 after MC-38 tumor cell inoculation to allow for a sufficient treatment window (Table 1).

TABLE 1
Treatment Groups

| | | Schedule (days | | Group size tumor- |
|---|---|---|---|---|
| Group | Test Article | post inoculation) | Route | bearing/tumor-naive |
| 1 | Vehicle | BID (10-37) | PO | 8/5 |
| 2 | 200 μg anti-PD-1 | BIW (10, 13, 17, 20) | IP | 8/5 |
| 3 | 50 mg/kg Compound 1 | BID (10-37) | PO | 8/5 |
| 4 | 50 mg/kg Compound 1 | BID (10-16) | PO | 8/5 |
| | 200 μg anti-PD-1 | BIW (10, 13, 17, 20) | IP | |
| 5 | 50 mg/kg Compound 1 | BID (10-23) | PO | 8/5 |
| | 200 μg anti-PD-1 | BIW (10, 13, 17, 20) | IP | |
| 6 | 50 mg/kg Compound 1 | BID (10-30) | PO | 8/5 |
| | 200 μg anti-PD-1 | BIW (10, 13, 17, 20) | IP | |
| 7 | 50 mg/kg Compound 1 | BID (10-37) | PO | 8/5 |
| | 200 μg anti-PD-1 | BIW (10, 13, 17, 20) | IP | |
| 8 | 50 mg/kg Compound 1 | BID (10-16/24-30) | PO | 8/5 |
| | 200 μg anti-PD-1 | BIW (10, 13, 17, 20) | IP | |

Anti-PD-1 was administered twice weekly (BIW) for 2 consecutive weeks by intraperitoneal (IP) injection at 200 μg/mouse. Compound 1 was administered twice daily (BID) for 28 consecutive days by oral gavage (PO) at 50 mg/kg in 10 mL/kg dose volume (with or without anti-PD-1 administered on the above indicated dose and schedule). Alternatively, Compound 1 was administered BID for 7, 14 or 21 consecutive days, or on Days 10-16 and Days 24-30 in combination with anti-PD-1. Concomitantly, a set of satellite cohorts comprising tumor-naïve mice was treated with the same schedules and doses as indicated above.

Animals were monitored for tumor growth and body weight 2-3 times per week based on outgrowth kinetics. Tumor diameter was measured with digital calipers, and the tumor volume in mm³ was calculated by the formula: Volume=((width)²×length)/2. Animals were removed from study if found moribund or if tumor volume exceeded 1500 mm³. Study results are displayed as mean change in tumor volume and body weight or survival. Food, water, temperature and humidity were according to Pharmacology Testing Facility performance standards (SOPs) which are in accordance with the 1996 Guide for the Care and Use of Laboratory Animals (NRC) and AAALAC-International.

Terminal necropsy from non-tumor-bearing cohorts occurred at 28 days from the start of dosing, and eyes from 4-5 animals in each group were harvested for histopathology assessment. Since not all terminal necropsy groups were dosed for the entirety of the 28-day dosing period, some groups effectively experienced a 1-3 week recovery period. Recovery sacrifice from tumor-bearing cohorts occurred at 28 days after the 28-day dosing period, and eyes from 5 animals in each group were harvested for histopathology assessment. Since not all recovery necropsy groups were dosed for the entirety of the 28-day dosing period, some groups effectively experienced an extended recovery period beyond the 28-day recovery period of 1-3 weeks. Briefly, animals were euthanized by CO₂ inhalation and eyes were harvested with forceps and placed in 1.5 mL modified Davidson's Fixative (Electron Microscopy Science, Cat. No. 64133-50, lot no. 190621-08) for up to 24 hours at room temperature. Tissues were then transferred to 1.5 ml 10% neutral buffered formalin (Thermo Scientific, cat. no. 9400-5, lot no. 435456) at room temperature. Fixed tissues were submitted Vet Path Services, Inc. (Mason, Ohio) for histological processing and microscopic evaluation.

Provantis™ pathology software v 10.1.0.1 was utilized for data capture and table generation. Histopathology grades were assigned as grade 1 (minimal), grade 2 (mild), grade 3 (moderate), grade 4 (marked), or grade 5 (severe) based on an increasing extent of change, unless otherwise specified. H&E stained slides were evaluated with standard light microscopy and epifluorescence (450-490 nm excitation).

### Tumor Model

The murine MC-38 colon adenocarcinoma cell line was developed in C57BL/6 mice upon subcutaneous injection of dimethylhydrazine (Corbett, T. H., Griswold, D. P., et al. (1975), Cancer Research 35, 2434-2439). Extensive immunogenomic, transcriptomic, and therapeutic background are available on this cell line (Yadav, M., Jhunjhunwala, S., et al. (2014) Nature, 515, 572-576; and Efremova, M., Rieder, D., et al. (2018) Nat. Commun. 9, 32). The MC-38 cell line is reported to harbor driver mutations in TP53, PTEN, SMAD2, SMAD4, ACVR2A, TGFB2, BRAF, AXIN, SOX9, and ARID1A. Furthermore, mutations in the MMR gene MSH3 indicate that this cell line is a model of human MSI/hypermutated colorectal cancer. Whole exome sequencing has also identified 1290 transcript coding variations (compared to the reference C57BL/6 genome), among which 170 were predicted to be MHC Class I-presented neo-epitopes.

MC-38 (National Cancer Institute-Frederick Tumor Repository, Frederick, Md.) cells were grown in a humidified atmosphere of 5% CO₂ using DMEM growth media (Gibco Life Technologies, 11965-092) supplemented with 10% fetal bovine serum (HyClone SH30071.03, Logan, Utah) and 100 U/mL penicillin, 100 μg/mL streptomycin (Gibco Life Technologies, 15140-122). Cells were confirmed murine virus and mycoplasma negative (IDEXX Laboratories Inc, Columbia, Mo.) prior to implantation.

### Study Comments and Protocol Deviations

TGI was calculated on Day 32, the last day for which the vehicle control arm was intact.
Among the tumor-bearing cohorts, Animals 4 and 8 in Group 3 were found dead and Animal 8 in Group 7 was moribund within the first week of dosing; these animals were redacted from analysis.
Among the tumor-naïve cohorts, Animal 5 in Group 5 and Animal 5 in Group 6 were found dead within the first week of dosing.
The study was terminated on Day 66. Any animals remaining with no palpable tumors were scored as cures.

### Analysis of Antitumor Efficacy and Tolerability

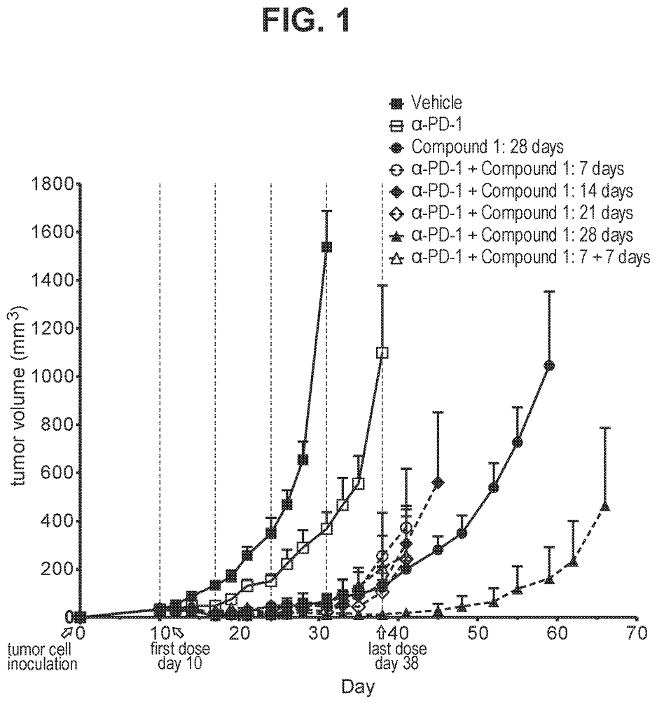

Data Analysis: The mean values for tumor volume (FIG. 1) by study day for each experimental group were plotted (error bars, SEM).

The following study metrics were evaluated and are summarized in Table 2 and Table 3):
Maximum % Body Weight Loss (% BWL): % BWL=100(1-BWₜ/BW₀); BW₀ is group mean body weight at study start and BWₜ is group mean or median body weight on day where maximal body weight loss is observed.
% Tumor Growth Inhibition (% TGI): % TGI=100(1−Wt/Wc); Wt is the mean tumor volume of the treated group on day X; Wc is the mean tumor volume of controls on day X, where X is the last day that the control group is available in its entirety.
Median Survival: Survival fractions were calculated with Prism Graph Software which utilizes the product limit method (Kaplan-Meier). Survival was defined as morbidity, mortality or tumor size exceeding 1500 mm³. Animals that were moribund or found dead for reasons unrelated to tumor or study drug administration (e.g. gavage trauma, etc.) were censored.
Cures: Animals with no palpable tumor on Day 66 were scored as cures.

TABLE 2
Study Metrics of Tolerability and Tumor Growth for Tumor-Bearing Cohorts

| | | | Maximum | | | | |
|---|---|---|---|---|---|---|---|
| | | Schedule | % Body | % TGI | Median | Cures | Deaths |
| | | (days post | Weight | on Day | Survival | on Day | (any |
| Group | Test Article | inoculation) | Loss | 32 | (days) | 66 | cause) |
| 1 | Vehicle | 10-37 | 1.24 | — | 31 | 0/8 | 0/8 |
| 2 | 200 μg | 10, 13, 17, | 2.14 | 76.1 | 45 | 0/8 | 0/8 |
| | anti-PD-1 | 20 | | | | | |
| 3 | 50 mg/kg | 10-37 | 5.17 | 95.6 | 66 | 0/6 | 2/8 |
| | Compound 1 | | | | | | |
| 4 | 50 mg/kg | 10-16 | 0.00 | 96.6 | not | 6/8 | 0/8 |
| | Compound 1 | | | | reached* | | |
| | 200 μg | 10, 13, 17, | | | | | |
| | anti-PD-1 | 20 | | | | | |
| 5 | 50 mg/kg | 10-23 | 0.00 | 97.1 | 66 | 3/8 | 0/8 |
| | Compound 1 | | | | | | |
| | 200 μg | 10, 13, 17, | | | | | |
| | anti-PD-1 | 20 | | | | | |
| 6 | 50 mg/kg | 10-30 | 2.98 | 98.5 | not | 3/8 | 0/8 |
| | Compound 1 | | | | reached* | | |
| | 200 μg | 10, 13, 17, | | | | | |
| | anti-PD-1 | 20 | | | | | |
| 7 | 50 mg/kg | 10-37 | 0.00 | 99.3 | not | 5/7 | 1/8 |
| | Compound 1 | | | | reached* | | |
| | 200 μg | 10, 13, 17, | | | | | |
| | anti-PD-1 | 20 | | | | | |
| 8 | 50 mg/kg | 10-16/24- | 0.23 | 96.2 | not | 4/8 | 0/8 |
| | Compound 1 | 30 | | | reached* | | |
| | 200 μg | 10, 13, 17, | | | | | |
| | anti-PD-1 | 20 | | | | | |

*More than 50% of animals remained alive at end of study

TABLE 3
Study Metrics of Tolerability and Tumor Growth for Tumor-Naïve Cohorts

| | | | Maximum % | |
|---|---|---|---|---|
| | | Schedule (days | Body Weight | Deaths |
| Group | Test Article | post inoculation) | Loss | (any cause) |
| 1 | Vehicle | 10-37 | 0.00 | 0/5 |
| 2 | 200 μg anti-PD-1 | 10, 13, 17, 20 | 0.00 | 0/5 |
| 3 | 50 mg/kg Compound 1 | 10-37 | 0.00 | 0/5 |
| 4 | 50 mg/kg Compound 1 | 10-16 | 0.00 | 0/5 |
| | 200 μg anti-PD-1 | 10, 13, 17, 20 | | |
| 5 | 50 mg/kg Compound 1 | 10-23 | 0.00 | 1/5 |
| | 200 μg anti-PD-1 | 10, 13, 17, 20 | | |
| 6 | 50 mg/kg Compound 1 | 10-30 | 0.00 | 1/5 |
| | 200 μg anti-PD-1 | 10, 13, 17, 20 | | |
| 7 | 50 mg/kg Compound 1 | 10-37 | 0.00 | 0/5 |
| | 200 μg anti-PD-1 | 10, 13, 17, 20 | | |
| 8 | 50 mg/kg Compound 1 | 10-16/24-30 | 0.00 | 0/5 |
| | 200 μg anti-PD-1 | 10, 13, 17, 20 | | |

### Results

### Tolerability

The doses and schedules employed in this study were well tolerated in all groups. Among the tumor-bearing cohorts, maximum body weight loss was <5.2%, with three deaths possibly attributed to test article administration. Among the tumor-naïve cohorts, body weight loss was not observed, with two deaths possibly attributed to test article administration. These data support the conclusion that Compound 1 is tolerated at these doses and schedules in the MC-38 murine model of colon adenocarcinoma.

### Efficacy

Anti-PD-1 monotherapy was minimally efficacious in this model, with two weeks of treatment resulting in 76.1% TGI, 45 days median survival (versus 31 days median survival for vehicle control), and 0/8 animals cured on Day 66 (versus 0/8 cures for vehicle control) (FIG. 1). Compound 1 as monotherapy dosed for 28 consecutive days was more efficacious, with twice daily treatment resulting in 95.6% TGI, 66 days median survival, and 0/6 animals cured. However, equivalent maximal efficacies were observed across all the combination therapy cohorts, particularly in cure rate. Compound 1 dosed for 7 days in combination with anti-PD-1 resulted in 96.6% TGI and 6/8 cures, while the interval dosing schedule (Compound 1 on Days 10-16 and Days 24-30) in combination with anti-PD-1 similarly resulted in 96.2% TGI and 4/8 cures. Compound 1 dosed for 14 days, 21 days, or 28 days in combination with anti-PD-1 resulted in 97.1%, 98.5%, and 99.3% TGI, respectively, and these three cohorts demonstrated cure rates of 3/8, 3/8, and 5/7, respectively. Finally, among these combination therapy cohorts, the median survival was not reached in any group except for the one dosed with Compound 1 for 14 days, which yielded a median survival of 66 days.

### Pathology

Terminal necropsy occurred at 28 days from the start of dosing. Since not all terminal necropsy groups were dosed for the entirety of the 28-day dosing period, some groups effectively experienced a 1-3 week recovery period. In vehicle control mice, melanin granules and rare autofluorescent granules were present within the retinal pigmented epithelial cell cytoplasm. The thickness of the outer nuclear layer (ONL) was 8-10 nuclei and the inner nuclear layer was 3-5 nuclei. Rods and cones did not autofluoresce under epifluorescent illumination at 450-490 nm more than the adjacent neural retina.

At terminal necropsy, test article-related microscopic changes were limited to multifocal swelling of the Rod and Cone processes in retina of mice administered Compound 1 for four weeks as monotherapy or in combination with anti-PD-1. This finding was only visualized under epifluorescent illumination and was characterized by scattered individual processes that were increased in diameter up to five times the surrounding processes, with increased autofluorescence. In minimally affected animals, a few affected processes were observed, while in mildly affected animals the swollen axons were more common. In the group treated with Compound 1 for four weeks as monotherapy, three animals were minimally affected and two animals were mildly affected. In the group treated with Compound 1 for four weeks in combination with anti-PD-1, four of five animals were minimally affected. There were no apparent effects on the outer nuclear layer, inner nuclear layer or retinal pigmented epithelium, although the presence of melanin in the retinal pigmented epithelium may have obscured findings.

Recovery sacrifice occurred at 28 days after the 28-day dosing period. Since not all recovery necropsy groups were dosed for the entirety of the 28-day dosing period, some groups effectively experienced an extended recovery period beyond the 28-day recovery period of 1-3 weeks. At recovery sacrifice, no definitively test article-related changes were observed. The only abnormal observation was unilateral focal atrophy of the outer nuclear layer in Animal 4 that was administered Compound 1 for four weeks in combination with anti-PD-1. This finding was located in the peripheral retina and was characterized by reduction of the outer nuclear layer to 1-2 nuclei. Given the focal and unilateral distribution of this change it was unlikely to be related to the test article.

### Example 2

### 28-Day Repeat Dose Oral Toxicity and Toxicokinetic Study of Compound 1 with a 28-Day Recovery Period in Sprague Dawley Rats

The objectives of this study were to examine the toxicological effects of Compound 1, assess the toxicokinetic profile, and to assess recovery from administration of Compound 1, when given by oral gavage QD to rats for 28 consecutive days. One hundred and ninety Sprague-Dawley rats (95 per sex) were utilized in this study. There were 4 dose groups of 15 animals per sex with each animal receiving an oral QD dose of a placebo (HPMCAS in 0.5% Affinisol™) or Compound 1 HCl at 30, 100, or 300/200 mg/kg/day for 28 days. Additionally, 10 rats/sex/dose level and 5 rats/sex/placebo control group served as toxicokinetic animals and received the placebo or Compound 1 HCl in the same manner and dosing volume as the main study groups.

Following the 28-day dosing period, selected animals were observed for a 28-day recovery period. Recovery animals were evaluated to assess reversibility of Compound 1-induced changes, if any were observed. Parameters evaluated include clinical observations, mortality and moribundity checks, body weights, food consumption, toxicokinetics, eye exams, serum chemistry, hematology, coagulation, urinalysis, gross pathology, organ weights, and microscopic pathology.

Following 28 days of once-daily oral administration of Compound 1 (30, 100, or 300/200 mg/kg), the toxicological response was characterized by, among others, structural/functional changes in the eyes (outer retinal atrophy; 100 mg/kg males and 30 mg/kg female; unrecoverable). Changes in the eyes and testes were considered to be adverse.

### Example 3

### A 7- or 14-Day Ocular Toxicology Study of Compound 1 in Female Sprague Dawley Rats with Variable Recovery Periods

### Objectives and Study Design

This study was designed to evaluate schedule-dependent ocular toxicity of Compound 1 in female Sprague Dawley rats and to determine if ocular toxicity findings occur and/or are reversible when dose administration is limited to 1 week or 2 weeks with 3 weeks and 2 weeks recovery, respectively (Day 28 toxicity endpoint). Overall tolerability was assessed by clinical observations and body weight, and an additional recovery period to Day 56 was also evaluated for histopathology of the eyes.

### SUMMARY

Female Sprague Dawley (N=68) rats were assigned to 4 treatment groups receiving placebo or Compound 1 HCl via oral gavage with a 10 mL/kg dose volume. Placebo, 100 mg/kg or 200 mg/kg Compound 1 HCl was administered once daily for 7 or 14 days, and eyes were harvested for histopathology assessment on day 28 or 56. The treatment was well tolerated with no effects on body weight, adverse clinical observations or deaths related to test article.

No histopathological effects were observed in the eyes of animals receiving 7 days treatment of 100 or 200 mg/kg once daily Compound 1 on Day 28. On Day 56, 1 of 5 animals had a unilateral equivocal change in the 100 mg/kg group and no changes were observed in the 200 mg/kg group. Based on these results, the NOAEL for once daily administration of Compound 1 for 7 days is 200 mg/kg.

There were dose-related ocular changes in animals receiving 14 days once daily Compound 1. Dose administration of 100 mg/kg resulted in 1/9 animals with ocular changes that were not recoverable (1/5 on day 56). At the high dose, 8/9 animals had ocular changes that were not recoverable (3/5 on day 56). Based on these results, the STD10 for 14 days once daily dose administration is 100 mg/kg.

### Methods

### Test Articles

Vehicle—0.5% HPMC, pH 5 (Affinisol HPMC HME 100 LV Hypromellose (HPMC, Dow Chemical Company ID99015561, Lot #INR477140)). To prepare, sterile water for injection was added gradually to HPMC with continuous agitation to create a clear solution.
Placebo—9.6% HPMCAS-M in vehicle. HPMCAS-M was spray-dried alone to be used as a placebo by Seran BioScience, Bend, Oreg., Lot No. DEV-009-037 Placebo. The dose formulation was prepared by suspending the white powder in vehicle. Dose suspensions were made one day before use. pH was checked before use and adjusted down if pH is >than 6 with 1N HCl.
Test Article Compound 1 HCl (Compound 1 HCl batch 28, 1:3 Compound 1 HCl:HPMCAS-M Spray-Dried Dispersion (SDD) prepared at Seran BioScience, Bend, Oreg., Seran Lot No. DEV-009-037, 22.9% (as Compound 1 HCl salt)). The SDD was prepared as a yellow suspension in vehicle. Dose suspensions were made one day before use. pH was checked before use and adjusted down if pH is >than 6 with 1N HCl.
200 mg/kg (20 mg/mL active=87 mg/mL SDD)
   Approximate amount needed:
   Week 1-30 female rats×0.3 kg/rat×200 mg/kg×1 QD×7 days×1.2 extra/0.23 active=65.74 g Compound 1-28
   Week 2-15 female rats×0.3 kg/rat×200 mg/kg×1 QD×7 days×1.2 extra/0.23 active=32.87 g Compound 1-28
   100 mg/kg Compound 1 HCl SDD dose suspension is pH 5.3
100 mg/kg (10 mg/mL active=43.5 mg/mL SDD)
   Approximate amount needed:
   Week 1-30 female rats×0.3 kg/rat×100 mg/kg×1 QD×7 days×1.2 extra/0.23 active=32.87 g Compound 1-28
   Week 2-15 female rats×0.3 kg/rat×100 mg/kg×1 QD×7 days×1.2 extra/0.23 active=16.44 g Compound 1-28
   200 mg/kg Compound 1 HCl SDD dose suspension is pH 4.97

### Experimental Animals

Female Sprague Dawley rat (N=68) from Envigo (Greenfield, Ind.), born on 9 Jul. 2019, were obtained at approximately seven weeks of age and housed in groups of 2-3. Food, water, temperature and humidity are according to Pharmacology Testing Facility performance standards (SOPs) which are in accordance with the 1996 Guide for the Care and Use of Laboratory Animals (NRC) and AAALAC-International. Animals were acclimated for 1 week then randomized into groups for dose administration according to protocol.

### Study Comments and Protocol Deviations

Group 4—one animal lost to gavage error as evidenced by rales. Group 5—one set of eyes lost at histology lab.

### Tissue Preparation

At predetermined times, eyes were collected into Davidson's fixative upon termination then transferred to neutral buffered formalin after 24 hrs. Eyes were submitted to Vet Path Services, Inc. following termination for sectioning and H&E staining. A veterinary ophthalmologist examined the sections and reported adverse effects.

### Results

### Tolerability

Female Sprague-Dawley rats were administered placebo or test article, Compound 1, once daily as a single agent by oral gavage (Table 4).

TABLE 4
Experimental Groups

| | | Dose | Route, | | | Recovery |
|---|---|---|---|---|---|---|
| Group | Compound | (mg/kg)^{a} | Schedule^{b} | Animal # | Necropsy | Period |
| 1 | Placebo | 0 | PO | 1-5 | Day 28 | 14 |
| | | | QD (1-14) | 6-8 | Day 56 | 42 |
| 2 | Compound 1 HCl | 100 | PO | 1-10 | Day 28 | 21 |
| | | | QD (1-7) | 11-15 | Day 56 | 49 |
| 3 | Compound 1 HCl | 200 | PO | 1-10 | Day 28 | 21 |
| | | | QD (1-7) | 11-15 | Day 56 | 49 |
| 4 | Compound 1 HCl | 100 | PO | 1-10 | Day 28 | 14 |
| | | | QD (1-14) | 11-15 | Day 56 | 42 |
| 5 | Compound 1 HCl | 200 | PO | 1-10 | Day 28 | 14 |
| | | | QD (1-14) | 11-15 | Day 56 | 42 |

^{a}100 mg/kg = ‘Test Article 1’; 200 mg/kg = ‘Test Article 2’
^{b}QD (1-7) = ‘Schedule 1’; QD (1-14) = ‘Schedule 2’

The drug regimen was well tolerated. No body weight effects or adverse clinical observations were noted, and no drug-related deaths were observed. One animal was terminated early due to gavage error as evidenced by rales.

Pathology Results

Terminal necropsy occurred on study day 28 for toxicology (‘in life’) groups. From terminal necropsy, eyes were evaluated from Group 1 (five control rats), Group 2 (10 rats administered Test article 1 on Schedule 1), Group 3 (10 rats administered Test Article 2 on Schedule 1), Group 4 (9 rats administered Test Article 1 on Schedule 2), and Group 5 (9 rats administered Test Article 2 on Schedule 2). In control rats, few, scattered, tiny round autofluorescent granules were present within the retinal pigmented epithelial cell cytoplasm. The thickness of the outer nuclear layer (ONL) was 8-10 nuclei and the inner nuclear layer was 3-5 nuclei. Rods and cones did not autofluoresce under epifluorescent illumination at 450-490 nm more than the adjacent neural retina.

No test article-related observations were made for rats administered Test Article 1 (Group 2) or Test Article 2 (Group 3) on Schedule 1.

In animals administered Test Article 1 (Group 4) on Schedule 2, one animal (4F-7) had moderate atrophy of the outer nuclear layer (ONL) of the retina with minimal diffuse degeneration of the rod and cone processes and minimal multifocal histiocytic infiltration of the rod and cone processes by granule-laden macrophages. Moderate atrophy of ONL was characterized by thinning of the layer to approximately 4-5 nuclei. Minimal degeneration of the rods and cones layer was characterized by increased diameter of the photoreceptor outer segments and was best visualized under epifluorescent illumination. Minimal infiltration of the rod and cone layer by macrophages laden with granules was characterized by individual macrophages within the rod and cone layer with distended cytoplasm filled with granules of pale eosinophilic material that were autofluorescent.

In animals administered Test Article 2 (Group 5) on Schedule 2, retinal changes were observed in eight animals. These changes consisted of minimal to moderate atrophy of the ONL of the retina, minimal or moderate degeneration of the rod and cone processes, and minimal to mild histiocytic infiltration of the rod and cone layer by granule-laden macrophages. Minimal atrophy of ONL occurred in two animals and was characterized by thinning of the layer to approximately 7-8 nuclei. Mild atrophy of ONL occurred in one animal and was characterized by thinning of the layer to approximately 6-7 nuclei. Moderate atrophy of ONL occurred in five animals and was as previously described. Minimal degeneration of the rods and cones layer in six animals was as previously described. Moderate degeneration of rod and cone processes was observed in two animals and was characterized by multifocal partial or complete collapse and disorganization of the rods and cones layer, and increased diameter of the photoreceptor outer segments. Minimal infiltration of the rod and cone layers was as previously described, and mild infiltration was characterized by accumulations of approximately 2 to 4 macrophages within the rod and cone layer.

Recovery necropsy occurred on study day 56 for toxicology (‘in life’) groups. From recovery necropsy, eyes were evaluated from Group 1 (three control rats), Group 2 (5 rats administered Test article 1 on Schedule 1), Group 3 (5 rats administered Test Article 2 on Schedule 1), Group 4 (5 rats administered Test Article 1 on Schedule 2), and Group 5 (5 rats administered Test Article 2 on Schedule 2). At recovery necropsy, test article-related changes in the eye were limited to the retina and consisted of atrophy of the ONL, atrophy of the rod and cone processes, and histiocytic infiltration of the retina by granule-laden macrophages. Test article-related retinal changes were observed only in animals administered Test Article 1 (Group 4) on Schedule 2 and in animals administered Test Article 2 (Group 5) on Schedule 2. Retinal changes were observed in one of five animals in Group 4 and three of five animals in Group 5. Of note, one animal from Group 2, had unilateral minimal diffuse atrophy of the ONL which was considered an equivocal change given that the finding was unilateral.

In one animal (4F-11) administered Test Article 1 (Group 4) on Schedule 2, moderate diffuse central atrophy of the ONL bilaterally was the only observation.

In animals administered Test Article 2 (Group 5) on Schedule 2, moderate diffuse central atrophy of the ONL bilaterally was observed in three animals with or without minimal or mild histiocytic infiltration of the ONL or rods and cones and/or focal moderate atrophy of the rod and cone processes in the central portion of the retina bilaterally.

Administration of Test Article 1 (Group 4) on Schedule 2 resulted in retinal changes with a low incidence. These findings did not reverse.

Administration of Test Article 2 (Group 5) on Schedule 2, resulted in retinal changes with a high incidence. These findings did not reverse.

### Example 4

### A Phase 1, Open-Label, Multi-Center, Dose-Finding, Pharmacokinetic, Safety and Tolerability Study of Compound 1 in Participants with Selected Advanced or Metastatic Solid Tumor Malignancies

### Rationale

This is a Phase 1, open-label, multi-center, multiple-dose, dose-escalation, safety, PK and biomarker study of Compound 1 in cohorts of adult participants with selected advanced or metastatic solid tumors for whom no standard therapy is available or in the opinion of the participant and their treating physician, that standard therapy would not be appropriate, or who have refused standard therapy. Successive cohorts of participants will receive escalating doses of Compound 1 on an outpatient basis starting from 25 mg QD.

### OBJECTIVES AND ENDPOINTS

| Objectives | Endpoints |
|---|---|
| Primary: | Primary: |
| To assess safety and tolerability of | Incidence of DLTs within the first 2 |
| increasing dose levels of Compound 1 | cycles. |
| in successive cohorts of participants | Adverse events as characterized by |
| with selected advanced or metastatic | type, frequency, severity (as graded by |
| solid tumors in order to estimate the | NCI CTCAE version 5.0), timing, |
| MTD and select the RP2D/schedule. | seriousness, and relationship to study |
| | therapy. |
| | Laboratory abnormalities as |
| | characterized by type, frequency, |
| | severity (as graded by NCI CTCAE |
| | version 5.0), and timing. |
| Secondary: | Secondary: |
| To characterize the single and/or | Pharmacokinetic parameters of |
| multiple dose PK of Compound 1 and | Compound 1 and its metabolite |
| its metabolite Compound 2*. | Compound 2*: single dose (SD) - Cₘₐₓ, |
| | Tₘₐₓ, AUCₗₐₛₜ, and as data permit, t_{1/2}, |
| | AUC_{inf}, AUCₜₐᵤ, CL/F, and V_{z/F}. |
| | Pharmacokinetic parameters of |
| | Compound 1 and its metabolite |
| | Compound 2*: multiple dose (MD) |
| | (assuming steady state is |
| | achieved) - C_{ss, max}, T_{ss, max}, AUC_{ss, tau}, |
| | and as data permit, CL/F, Vₛₛ/F, and |
| | R_{ac} (AUC_{ss, tau}/AUC_{sd, tau}). |
| To evaluate preliminary anti-tumor | OR, as assessed using the RECIST |
| activity of Compound 1. | version 1.1. |
| | Time-to-event endpoints: e.g., DOR |
| | and DCR. |

*N-(4-((3-amino-1H-pyrazolo[3,4-b]pyridin-4-yl)oxy)-3-fluorophenyl)-3-(4-fluorophenyl)-1-isopropyl-2,4-dioxo-1,2,3,4-tetrahydropyrimidine-5-carboxamide (disclosed in WO 2020/047184 A1, Example 196)

### BACKGROUND

It has been demonstrated that Compound 1 lowers the threshold for immune activation and promotes anti-tumor immunity using multiple syngeneic tumor models where robust T cell-dependent anti-tumor immunity is seen (Examples 1 and 2). In addition, tumor-bearing mice that were cured following treatment with Compound 1 were shown to be resistant to subsequent re-challenge with the same syngeneic tumor, demonstrating that AXL/MERTK inhibition leads to the generation of long-term anti-tumor T cell memory effectors. Inhibition of MERTK and AXL with Compound 1 therefore has the potential to increase anti-tumor immunity in solid tumor types that are known to be responsive to immunotherapy and have single agent activity and it could demonstrate added clinical benefit in certain combination therapy regimens.

### Study Design

The purpose of this first-in-human study is to assess the safety, tolerability, PK and preliminary activity of Compound 1 in participants with selected advanced or metastatic solid tumors.

Successive cohorts of participants will receive escalating doses of Compound 1 on an outpatient basis starting from 25 mg QD.

Approximately 27 participants evaluable for MTD/RP2D determination will be enrolled. The total number of participants will depend on the number of dose levels needed to determine the MTD and number of participants evaluable for DLT at each dose level. A participant is classified as evaluable if he/she experiences a DLT or if he/she, in the absence of a DLT, receives at least 75% of the planned doses of the study intervention during each of Cycle 1 and 2 and has completed all scheduled safety assessments during the DLT window. For the purpose of dose escalation, the DLT observation period will encompass the first 2 cycles of treatment (i.e., 42 days) for each participant.

Treatment with study intervention will continue until either disease progression, participant refusal, or unacceptable toxicity occurs, whichever occurs first.

One initial schedule of administration will be explored. Each cycle will be 21 days in duration, with QD dosing of Compound 1 on a 14-day schedule (Day 1 to Day 14) followed by a 7-day dosing holiday (Day 15 to Day 21; i.e., 2 weeks on/1 week off).

The proposed doses, schedule, and PK time points may be reconsidered and amended during the study based on the emerging safety and PK data.

This clinical study consists of dose escalation. As data are acquired, the protocol may be amended to include a dose-expansion portion. The dose escalation will estimate the MTD/RP2D of single-agent Compound 1 in dose-escalation cohorts in participants with selected advanced or metastatic solid tumors.

In the dose escalation, a BLRM will be used to model the relationship of DLTs to the dose of Compound 1. This model, along with EWOC, will guide the dose escalation of Compound 1 after the completion of the DLT observation period of each cohort, until the determination of MTD. After all patients in a cohort have completed the DLT observation period, a dose-escalation meeting will be scheduled by the sponsor with the investigators. The DLTs, along with the safety, PK and other relevant data will be reviewed in detail and a decision will be made about the dose for the next cohort. Cohorts of a minimum of 3 evaluable participants will be treated at each dose level of Compound 1 until the determination of MTD. Enlargement of the MTD cohort will determine/confirm the RP2D, with a minimum of 6 participants with selected advanced or metastatic solid tumors expected to be treated at MTD/RP2D.

Toxicities will only be considered DLTs if they occur within the DLT window of the first 2 cycles (42 days); however, overall safety, including later cycles, and PK data will be evaluated for the RP2D determination. If a dose lower than the MTD is under consideration for the RP2D, additional participants may be dosed at this potential RP2D to ensure the safety and biomarker/PK data are evaluated in a sufficient number of participants.

The biomarker studies will be used to help understand the in vivo mechanism of action of the agent(s) studied as well as potential mechanisms of resistance. The studies may help in the future development of Compound 1 as a single agent, or in combination with other compounds, and may provide information on tumor sub-types that may respond to the study intervention.

### Scientific Rationale for Study Design

Compound 1, acting through AXL/MERTK, functions as an 10 enhancer. Through expression of MERTK and AXL by myeloid and dendritic cells, inhibition of these receptors by Compound 1 is expected to induce in anti-tumor immunity via 1) increasing T cell priming (immunogenic cell death) and 2) reversing an immune suppressive tumor microenvironment (M2 macrophages and MDSCs).

Tumor types of cervical, gastric, esophageal, HCC, melanoma (mucosal or cutaneous), Merkel cell, MSI-H tumors, NSCLC, HNSCC, SCLC, RCC and urothelial were selected based on known responsiveness to immunotherapy. Given the known mechanism of action of Compound 1, patients with these tumor types are most likely to receive benefit from Compound 1.

Nonclinical studies (e.g., Examples 1 and 2) have demonstrated that shorter, intermittent durations of dose administration eliminate and/or decrease the incidence and severity of on-target, retinal toxicity in dogs, rats, and mice, and anti-tumor studies have shown that shorter, intermittent durations of dose administration provide equivalent anti-tumor immunity in syngeneic tumor models. Thus, the proposed dosing duration of Compound 1 in this first-in-human study (intermittent schedule of 2 weeks on/1 week off) is intended to provide for substantially reduced risk of ocular findings while maintaining the potential for maximum anti-tumor activity.

Compound 1 has been identified to be primarily metabolized by CYP3A4 and CYP2D6 in vitro. CYP genotyping to identify poor- and extensive-metabolizers of CYP2D6 substrates will be included in this study for post-hoc analyses.

Outer retinal atrophy occurred in rats receiving continuous daily dosing of Compound 1 at HEDs that were approximately 12 to 39 times greater than the planned initial dose in humans. Similar findings appeared in dogs, although the severity was modest as compared to the rat data. When intermittent dosing (shorter dose administration intervals [1 to 3 weeks]) with a recovery period of 1 week) was employed, the therapeutic multiples were 39-78 when compared to expected exposure at the starting dose of 25 mg QD. For clinical consideration in this study, participants will be advised when outdoors in daylight to wear sunglasses with UV-reducing coating to protect the eyes. Ophthalmic examinations will be performed at regular intervals. The ophthalmic evaluation should be repeated at any point during the treatment period, if clinically indicated. Participants will be instructed to report new visual changes immediately.

### Criteria for Dose Escalation

Bayesian logistic regression model (BLRM) guided by the EWOC (escalation with overdose control) principle will be used in dose escalation. Using DLT data at all tested dose levels and pre-specified prior distribution of model parameters, posterior probabilities of probability of having a DLT falling into three dosing intervals (underdosing, target dosing, overdosing) will be calculated for all dose levels. A dose may only be used for newly enrolled participants if the risk of excessive toxicity, i.e., toxicity higher than 0.33 at that dose is less than 25%.

The provisional dose levels to be evaluated are listed in Table 5. Doses and dosing schedules beyond those listed in Table 5 may be permitted at the discretion of the sponsor. Dose escalation will stop when stopping criteria are met; i.e., at least 15 participants have been treated and at least 6 participants have been treated at the MTD/RP2D.

TABLE 5
Provisional Dose Levels in Dose-Escalation

| | Compound 1 | Dosing Schedule |
|---|---|---|
| Cohort | Dose (mg, QD) | (Cycle length = 21 days) |
| 1 | 25 | 2 weeks on/1 week off |
| (starting dose) | | |
| 2 | 50 | |
| 3 | 100 | |
| 4 | 200 | |
| 5 | 300 | |

### Dose-Limiting Toxicity (DLT) Definition

A participant is classified as DLT-evaluable if he/she experiences a DLT or if he/she otherwise in the absence of a DLT receives at least 75% of the planned doses of the study intervention during each of Cycle 1 and 2 and has received all scheduled safety assessments during the DLT window. If a participant fails to meet these criteria, he/she may be replaced. For the purpose of dose escalation, the DLT observation period will be during the first 2 cycles of treatment (i.e., 42 days) in each participant.

If accumulated safety and PK data from ongoing participants suggest, the DLT observation period may be reduced to 1 cycle (21 days) and the definition of DLT evaluability will be adapted accordingly, with a change in the requirement of 75% of the planned dose in Cycle 1 only. The intent of changing this feature of the design is to be able to provide opportunities for participants to be treated with higher and potentially more effective doses more rapidly. Upon this transition to a 21-day DLT observation period, participants who discontinue treatment before completing Cycle 1 or receive less than 75% of the planned doses during this new DLT window will be replaced. This potential change to a new DLT window will only occur at the beginning of a new dose escalation cohort.

Similarly, significant adverse events (AEs) considered to be related to the study intervention or treatment under investigation that occur after the DLT observation period will be reviewed in context of all safety data available. That review may result in re-evaluation of the dosing level or regimen.

In both situations, the sponsor will schedule a meeting with the investigators and determine if the DLT observation period should be reduced, or, in the case of late toxicity, if the enrollment should be held, continued, or if a dose reduction should be implemented for all ongoing participants.

Severity of AEs will be graded according to CTCAE version 5.0.

DLTs are as follows:

### Hematological:

Grade 4 neutropenia lasting >7 days;
Grade ≥3 neutropenia with infection;
Febrile neutropenia (defined as ANC <1000/mm³ with a single temperature of >38.3° C. [101° F.], or a sustained temperature of ≥38° C. [100.4° F.] for more than one hour);
Grade 3 thrombocytopenia with ≥Grade 2 (clinically significant) bleeding. For bleeding events with no grading available, clinically significant bleeding is defined as requiring hospitalization or urgent medical intervention;
Grade 2 clinically significant bleeding. For bleeding events with no grading available, clinically significant bleeding is defined as requiring hospitalization or urgent medical intervention.
Grade 4 thrombocytopenia.

### Non-Hematologic:

Grade ≥3 toxicities that are clinically significant, except those that have not been maximally treated (e.g., nausea, vomiting, diarrhea) or can be easily treated (e.g., electrolyte abnormalities);
For participants with Grade 2 hepatic transaminase or alkaline phosphatase levels at baseline as a result of liver metastasis or bone metastasis, a hepatic transaminase or alkaline phosphatase level >10×ULN;
Confirmed DILI meeting Hy's law criteria (ALT/AST >3× the ULN with bilirubin >2×ULN without another explanation (e.g., cholestasis);
Grade 3 fatigue lasting ≥5 days;
Grade 3 diarrhea for ≥48 hours despite optimal use of antidiarrheal therapy, Grade 4 diarrhea, Grade 3 nausea/vomiting for ≥48 hours despite optimal use of antiemetic therapy, or Grade 4 nausea and vomiting;
Grade 3 skin toxicity (e.g., rash, dermatitis, hand foot skin reaction) for >14 consecutive days despite maximal skin toxicity treatment (as per local practice), or Grade 4 rash and/or hand foot skin reaction;
Grade 2 eye disorders assessed to be irreversible or unresponsive to local therapy within 21 days and confirmed by ophthalmic evaluation, or Grade eye disorders confirmed by ophthalmic evaluation;
In an asymptomatic participant, Grade 3 QTc prolongation will first require repeat testing, re-evaluation by a qualified person, and correction of reversible causes such as electrolyte abnormalities or hypoxia for confirmation. If, after correction of any reversible causes, the Grade 3 QTc prolongation persists;
Clinically important or persistent toxicities (e.g., toxicities responsible for weeks dose delay) that are not included in the above criteria may also be considered a DLT following review by the investigators and the sponsor. All DLTs need to represent a clinically significant shift from baseline.
   The following AEs will not be adjudicated as DLTs:
Isolated Grade 3 or 4 laboratory abnormalities that are not associated with clinical sequelae and are corrected with supplementation/appropriate management within 72 hours of their onset.

Post-DLT, patients deriving clinical benefit from study treatment may continue on study at a reduced dose (see Table 7) following recovery of the AE to Grade ≤1 or baseline, only after discussion between the investigator and sponsor.

### Maximum Tolerated Dose (MTD) Definition

MTD is defined as a highest dose with probability of DLT from the target toxicity interval. The target interval for the DLT rate is defined as (0.16, 0.33).

For any given participant that is on-treatment at dose levels that are subsequently considered to be above the MTD, the option to dose reduce will be discussed. If a participant is tolerating the above MTD dose level and is benefiting from therapy, continuation of treatment at the above MTD dose level will require re-consenting.

### Recommended Phase 2 Dose Definition

The RP2D is the dose chosen for further investigation based on Phase 1 study results. If the MTD proves to be clinically feasible for long term administration in a reasonable number of participants, then this dose usually becomes the RP2D. Further experience with the MTD may result in a RP2D dose lower than the MTD. During escalation and prior to an MTD being reached, the sponsor may choose a lower dose than the MTD as the RP2D, particularly (but not exclusively) when considering potential future combination cohorts. This decision may be made based on safety, PK, and/or efficacy.

### Inclusion Criteria

Participants are eligible to be included in the study only if all of the following criteria apply:
1. Females and/or male participants age ≥16 years.
2. Histological or cytological diagnosis of cervical cancer, gastric cancer, esophageal cancer, HCC, melanoma (mucosal or cutaneous), Merkel cell carcinoma, MSI-H tumors, NSCLC, HNSCC, SCLC, RCC, or urothelial carcinoma that is resistant to standard therapy or for which no standard therapy is available.
3. Participants with measurable or non-measurable lesion(s) that has not been previously irradiated, as defined by RECIST version 1.1.

### 4. ECOG PS 0 or 1.

5. Adequate Bone Marrow Function, including:
a. ANC ≥1,500/mm³ or ≥1.5×10⁹/L;
b. Platelets ≥100,000/mm³ or ≥100×10⁹/L; ≥60×10⁹/L for HCC
c. Hemoglobin ≥9 g/dL (transfusion support is permitted if completed >1 month prior to planned start of dosing).
6. Adequate Renal Function, including:
a. Estimated creatinine clearance ≥60 mL/min as calculated using the method standard for the institution. In equivocal cases, a 24 hour urine collection test can be used to estimate the creatinine clearance more accurately.
7. Adequate Liver Function, including:
b. Total serum bilirubin ≤1.5×ULN unless the participant has documented Gilbert syndrome; ≤2.5×ULN for HCC;
c. AST and ALT≤2.5×ULN; ≤5.0×ULN if there is liver involvement by the tumor; ≤5.0× ULN for HCC;
d. Alkaline phosphatase ≤2.5×ULN (≤5×ULN in case of bone metastasis).
8. For HCC only:
a. Childs-Pugh cirrhotic status A or B with a maximum score of 7.
b. No evidence of clinically apparent ascites or active encephalopathy, and/or varices that have not been treated. Participants with controlled ascites or encephalopathy are eligible so long as they meet Childs-Pugh score criterion. Controlled ascites and encephalopathy require scores of 2 each when calculating the Childs-Pugh score.
9. Resolved acute effects of any prior therapy to baseline severity or CTCAE Grade ≤1 except for AEs not constituting a safety risk by investigator judgment and discussion with the sponsor. Note: Stable chronic conditions (≤Grade 2) that are not expected to resolve (e.g., neuropathy, myalgia, alopecia, prior therapy-related endocrinopathies) are exceptions and participants with these conditions may enroll.
10. Participants who are willing and able to comply with all scheduled visits, treatment plan, laboratory tests, lifestyle considerations, and other study procedures.
11. Able to provide archival tumor tissue (a FFPE tumor tissue block or 10 unstained slides) from a primary or metastatic lesion for central laboratory testing. In the event tumor tissue is not available; the participant may enroll upon approval from the sponsor.
12. Able to swallow Compound 1 capsules.
13. Capable of giving signed informed consent as described in Appendix 1, which includes compliance with the requirements and restrictions listed in the ICD and in this protocol.

### Exclusion Criteria

Participants are excluded from the study if any of the following criteria apply:
1. Participants with known symptomatic brain metastases requiring steroids. Participants with previously diagnosed brain metastases are eligible if they have completed their treatment and have recovered from the acute effects of radiation therapy or surgery prior to study entry, have discontinued corticosteroid treatment for these metastases for at least 4 weeks and are neurologically stable for 3 months (requires MRI confirmation).
2. Participants with any other active malignancy within 2 years prior to enrollment, except for adequately treated basal cell or squamous cell skin cancer, or carcinoma in situ.
3. Major surgery within 6 weeks prior to study entry.
4. Radiation therapy within 4 weeks prior to study entry.
5. Last anti-cancer treatment within 2 weeks or 5 half-lives (whichever is shorter), unless the last immediate anti-cancer treatment contained an antibody-based agent(s) (approved or investigational), then the interval of 4 weeks or 5 half-lives (whichever is shorter) is required prior to receiving the study intervention. Participation in other studies involving investigational drug(s) within 4 weeks or 5 half-lives (whichever is shorter) prior to study enrollment.
6. Prior irradiation to >25% of the bone marrow.
7. Active autoimmune disease requiring immunosuppressive treatment or history of autoimmune disease requiring immunosuppressive therapy (e.g., requirement for systemic therapy with >10 mg/day prednisone-equivalent) or any other concurrent use of immunosuppressive therapy.
8. Participants with active, uncontrolled bacterial, fungal, or viral infection, including HBV, HCV, known HIV or AIDS-related illness. Participants with HBV, HCV, known HIV or AIDS-related illness are allowed on a case-by-case basis after discussion with sponsor's medical monitor regarding, but not limited to, the following criteria:
a. Participant's overall immune status; e.g., for HIV-positive subjects, current and past CD4 and T-cell counts, history (if any) of AIDS-defining conditions (e.g., opportunistic infections), and status of HIV treatment;
b. Participants should not receive anti-viral agents which are moderate or strong CYP3A and CYP2D6 inducers/inhibitors and the potential for other drug-drug interactions will be taken into consideration.
9. Participants meeting any of the following ophthalmic-related criteria, including:
a. Active retinal pigment epithelium/photoreceptor disorders (e.g., retinitis pigmentosa, cone-rod dystrophies, macular degeneration, etc.).
b. Known previous or current serious ophthalmic disease, history of cataract surgery within <8 days, serious eye trauma, intraocular or ocular surgery other than refractive surgery (i.e., lasik, cataract).
c. Participants with glaucoma, history of glaucoma or family history of inherited retinal or optic nerve disorders.
d. On medications that could affect the OCT (e.g., chloroquine or miotics).
e. Best-corrected visual acuity worse than 20/40 in either eye.
f. Refractive error in either eye exceeding+6 D (sphere) or +2.5 D (cylinder).
g. Participants with amblyopia.
h. Clinically significant abnormal findings in either eye on ophthalmic assessments including external eye examination, biomicroscopy, IOP >22 mm Hg, fundoscopy, fundus autofluorescence, OCT or visual fields. Repeat any test if there is a finding that is uncertain.
10. Baseline 12 lead ECG that demonstrates clinically relevant abnormalities that may affect participant safety or interpretation of study results (e.g., baseline QTc interval >470 msec, complete LBBB, signs of an acute or indeterminate age myocardial infarction, ST-T interval changes suggestive of active myocardial ischemia, second or third degree AV block, or serious bradyarrhythmias or tachyarrhythmias). If the baseline uncorrected QT interval is >470 msec, this interval should be rate corrected using the Fridericia method and the resulting QTcF should be used for decision making and reporting. If QTc exceeds 470 msec, or QRS exceeds 120 msec, the ECG should be repeated 2 more times and the average of the 3 QTc or QRS values should be used to determine the participant's eligibility. Computer interpreted ECGs should be overread by a physician experienced in reading ECGs before excluding participants. Cases must be discussed in detail with sponsor's medical monitor to judge eligibility.
11. Any of the following in the previous 6 months: myocardial infarction, long QT syndrome, Torsade de Pointes, arrhythmias (including sustained ventricular tachyarrhythmia and ventricular fibrillation), serious conduction system abnormalities (e.g., left anterior hemiblock), unstable angina, coronary/peripheral artery bypass graft, symptomatic CHF, New York Heart Association class III or IV, cerebrovascular accident, transient ischemic attack, symptomatic pulmonary embolism, and/or other clinical significant episode of thromboembolic disease. Ongoing cardiac dysrhythmias of NCI CTCAE≥Grade 2, atrial fibrillation of any grade (≥Grade 2 in the case of asymptomatic lone atrial fibrillation). If a participant has a cardiac rhythm device/pacemaker placed and QTcF>470 msec, the participant can be considered eligible. Participants with cardiac rhythm device/pacemaker must be discussed in detail with sponsor's medical monitor to judge eligibility.
12. Anticoagulation with vitamin K antagonists or factor Xa inhibitors is not allowed. Anticoagulation with subcutaneous heparin is allowed.
13. Hypertension that cannot be controlled by medications (i.e., >150/90 mmHg) despite optimal medical therapy.
14. Known or suspected hypersensitivity to any component of the study treatment or excipients.
15. Participants taking any prohibited concomitant medication or those unwilling/unable to switch to permitted concomitant medication.
16. Active inflammatory gastrointestinal disease, chronic diarrhea, known diverticular disease or previous gastric resection or lap band surgery. Gastroesophageal reflux disease under treatment with proton pump inhibitors is allowed (assuming no drug interaction potential).
17. Active bleeding disorder, including gastrointestinal bleeding, as evidenced by hematemesis, significant hemoptysis or melena in the past 6 months.
18. Other medical or psychiatric condition including recent (within the past year) or active suicidal ideation/behavior or laboratory abnormality that may increase the risk of study participation or, in the investigator's judgment, make the participant inappropriate for the study.
19. Investigator site staff or Pfizer employees directly involved in the conduct of the study, site staff otherwise supervised by the investigator, and their respective family members.

### Lifestyle Considerations

Participants will be advised to report any visual changes immediately. In addition, special precautions will be taken to limit any potential eye irritation effect, by minimizing the participants' exposure to light including sunlight, and high intensity UVB light sources such as tanning beds, tanning booths and sunlamps. Participants should be encouraged to wear sunglasses with UV-reducing coating to protect the eyes when outdoors in daylight.

### Contraception

The investigator or his or her designee, in consultation with the participant, will confirm that the participant has selected an appropriate method of contraception for the individual participant and his or her partner(s) from the permitted list of contraception methods and will confirm that the participant has been instructed in its consistent and correct use. At time points, the investigator or designee will inform the participant of the need to use highly effective contraception consistently and correctly and document the conversation and the participant's affirmation in the participant's chart (participants need to affirm their consistent and correct use of at least 1 of the selected methods of contraception). In addition, the investigator or designee will instruct the participant to call immediately if the selected contraception method is discontinued or if pregnancy is known or suspected in the participant or partner.

### Screen Failures

Screen failures are defined as participants who consent to participate in the clinical study but are not subsequently enrolled in the study. A minimal set of screen failure information is required to ensure transparent reporting of screen failure participants to meet the CONSORT publishing requirements and to respond to queries from regulatory authorities. Minimal information includes demography, screen failure details, eligibility criteria, and any SAE.

Individuals who do not meet the criteria for participation in this study (screen failure) may be rescreened once at the discretion of the Investigator and/or individual screening assessments may be repeated, as appropriate. If a particular screening assessment is repeated, the results obtained closest to the first dose of study intervention should be used to assess eligibility.

### Study Intervention

Study intervention is defined as any investigational intervention(s), marketed product(s), placebo, medical device(s), or study procedure(s) intended to be administered to a study participant according to the study protocol.

For the purposes of this protocol, study intervention refers to Compound 1.

Study Intervention(s) Administered

| | |
|---|---|
| Intervention Name | Compound 1 |
| Type | Drug |
| Dose Formulation | Capsule |
| Unit Dose Strength(s) | 5 mg, 25 mg, 50 mg |
| Dosage Level(s) | See Table for dose levels and frequency |
| Route of Administration | Oral |
| Use | Experimental |
| IMP or NIMP | IMP |
| Sourcing | Compound 1 will be provided centrally by the sponsor |
| Packaging and Labeling | Compound 1 will be provided open-label in high-density |
| | polyethylene bottles. Each bottle will be labeled as required |
| | per country requirement. |

Compound 1 will be provided as capsules for oral administration. The 5 mg, 25 mg and 50 mg capsules will be supplied in separate bottles and labeled according to local regulatory requirements.

### Administration

Participants will receive Compound 1 according to the dose cohort assigned at enrollment. Participants will continue to receive Compound 1 until they meet the protocol-defined criteria for treatment discontinuation. For an individual participant, the dose of study treatment may be reduced or interrupted as appropriate based on protocol-defined treatment modifications.
Compound 1 dosing will begin on Cycle 1 Day 1.
A cycle is defined as the time from Day 1 dose to the next Day 1 dose. If there are no treatment delays, a cycle will be 21 days.
Participants will take the Compound 1 QD for 14 days (2 weeks), followed by one week off.
On study visit days, and on days when PK samples will be drawn, the Compound 1 will be administered to the participants at the study site.
Other than the above-mentioned study visits, participants will self-administer the Compound 1.
Participants should be instructed to swallow Compound 1 whole, and not manipulate or chew the capsule prior to swallowing. Participants should be instructed to not open the capsules.
Oral Compound 1 will be administered QD with at least 8-oz (240 mL) of water on an empty stomach. No food or liquids other than water will be consumed for ≥2 hours before (≥8 hours before on PK days in Cycles 1 and 2) and 2 hours following each dose throughout the study. Compound 1 should be taken daily in the morning at approximately the same time (±2 hours) every day.
If a participant misses a day of treatment, he/she must be instructed not to “make it up” but to resume daily dosing the next calendar day as prescribed.
If a participant vomits any time after taking a dose, he/she must be instructed not to take a second dose that calendar day but to resume daily dosing the next calendar day as prescribed.
If a participant inadvertently takes 1 extra dose during a day, the participant should not take the next scheduled dose of Compound 1.

### Concomitant Therapy

Concomitant treatment considered necessary for the participant's well-being may be given at discretion of the treating physician.

All concomitant treatments, blood products, as well as nondrug interventions (e.g., paracentesis) received by participants from screening until the end of treatment visit will be recorded on the CRF.

All concomitant treatments must be approved by the sponsor at study entry and during Cycle 1.

### CYP Substrates and Inhibitors

Because inhibition of CYP3A4/5 isoenzymes may increase Compound 1 exposure leading to potential increases in toxicities, the use of known strong or moderate inhibitors is not permitted within 10 days or 5 half-lives of the CYP3A4/5 inhibitors, whichever is longer, prior to the first dose of study intervention. Examples of strong CYP3A4/5 inhibitors include grapefruit juice or grapefruit/grapefruit related citrus fruits (e.g., Seville oranges, pomelos), ketoconazole, miconazole, itraconazole, voriconazole, posaconazole, clarithromycin, telithromycin, indinavir, saquinavir, ritonavir, nelfinavir, amprenavir, fosamprenavir, nefazodone, lopinavir, troleandomycin, mibefradil, and conivaptan. Examples of moderate CYP3A4/5 inhibitors include aprepitant, ciprofloxacin, conivaptan, crizotinib, cyclosporine, diltiazem, dronedarone, erythromycin, fluconazole, fluvoxamine, imatinib, tofisopam, and verapamil.

Because induction of CYP3A4/5 isoenzymes may decrease Compound 1 exposure leading to potential decrease in efficacy, the use strong CYP3A4/5 inducers is not permitted within 28 days or 5 half-lives of CYP3A4/5 inducers, whichever is longer prior to the first dose of study intervention. Examples of strong CYP3A4/5 inducers include phenobarbital, rifampin, phenytoin, carbamazepine, rifabutin, rifapentin, clevidipine, and St. John's Wort.

Because inhibition of CYP2D6 isoenzymes may increase Compound 1 exposure leading to potential increases in toxicities, the use of known strong or moderate inhibitors is not permitted within 10 days or 5 half-lives of CYP2D6 inhibitors, whichever is longer, prior to the first dose of study intervention. Examples of strong CYP2D6 inhibitors include quinidine, fluoxetine, paroxetine and bupropion. Examples of moderate CYP2D6 inhibitors include cimetidine, cinacalcet, duloxetine, fluvoxamine, and mirabegron.

Concomitant use of Compound 1 and a CYP2C9 substrate may increase the exposure of the CYP2C9 substrate. Examples of CYP2C9 substrates with a narrow therapeutic index include warfarin, phenytoin, glimepiride, glipizide, glyburide, ibuprofen, diclofenac, indomethacin, naproxen, rosiglitazone, sulfamethaxazole, tolbutamide, candesartan, irbesartan, lasartan and valsartan. Therefore, caution is warranted with coadministration of these and other CYP2C9 substrates.

Concomitant use of Compound 1 and a CYP3A4/5 substrate may increase the exposure of the CYP3A4/5 substrate. Examples of CYP3A4/5 substrates with a narrow therapeutic index include astemizole, terfenadine, cisapride, pimozide, quinidine, tacrolimus, cyclosporine, sirolimus, (alfentanil and fentanyl, excluding transdermal patch), and ergot alkaloids (ergotamine, dihydroergotamine). Therefore, caution is warranted with coadministration of these CYPA4/5 substrates.

### Hormonal Contraceptives

Concomitant use of Compound 1 and hormonal contraceptives that are CYP3A4/5 substrates may result in decreased exposure to hormonal contraceptives and may reduce effectiveness. Examples of hormonal contraceptives that are CYP3A4/5 substrates include ethinyl estradiol and progestin. For participants using hormone-based, highly effective methods of contraception with low user dependency, including hormonal-based implants and intrauterine devices, an effective barrier method must also be used.

### Antacid Medications

The aqueous solubility of Compound 1 is pH dependent. Therefore, antacid medications (including proton pump and H2 antagonists) may decrease the absorption of Compound 1. The use of antacid medications is prohibited.

### Transporter Substrates and Inhibitors

Compound 1 is a substrate of P-gp and BCRP in vitro. Inhibitors and inducers of these transporters should be used with caution. P-gp inhibitors include amiodarone, carvedilol, clarithromycin, dronedarone, itraconazole, lapatinib, lopinavir, propafenone, quinidine, ranolazine, ritonavir, saquinavir, telaprevir, tipranavir and verapamil. BCRP inhibitors include curcumin, cyclosporine A, and eltrombopag. Compound 1 is also an inhibitor of P-gp; therefore, sensitive P-gp substrates should be used with caution.

### Other Anti-Tumor/Anti-Cancer or Experimental Drugs

No additional anti-tumor treatment will be permitted while participants are receiving study treatment. Additionally, the concurrent use of select vitamins or herbal supplements is not permitted.

### Hematopoietic Growth Factors

Primary prophylactic use of colony stimulating factors is not permitted during the first 2 cycles of treatment (i.e., 42 days), but they may be used to treat treatment-emergent neutropenia as indicated by the current ASCO guidelines.8 During the screening window (i.e., 28 days prior to Day 1), granulocyte colony-stimulating factors are not permitted to qualify a participant with low WBC counts.

Erythropoietin may be used at the investigator's discretion for the supportive treatment of anemia.

### Anti Diarrheal, Anti Emetic Therapy

Primary prophylaxis beyond the first cycle is at the investigator's discretion. The choice of the prophylactic drug as well as the duration of treatment is up to the investigator with sponsor approval assuming there is no known or expected drug-drug interaction and assuming the drug is not included in the list of drugs under the heading “Concomitant Therapy” in this Example.

### Anti Inflammatory Therapy

Anti-inflammatory or narcotic analgesic use may be offered as needed assuming there is no known or expected drug-drug interaction and assuming the drug is not included in the list of drugs under the heading “Concomitant Therapy” in this Example.

### Corticosteroids

Chronic systemic corticosteroid use (prednisone >10 mg/day or equivalents) for palliative or supportive purposes is not permitted. Short-term, low-dose corticosteroid (e.g., 5 mg QD of prednisone or equivalent, for 2 weeks) use is permitted as symptomatic treatment on an individual basis and upon discussion with the sponsor. Acute administration, topical applications, inhaled sprays, eye drops, or local injections of corticosteroids are allowed. Under conditions of an AE requiring hold of study intervention, steroid use, if required, is allowed to treat the AE until the AE has resolved. Once the steroid dose has been tapered down to a low dose or off, study intervention may be resumed if permitted per Table 7.

### Surgery

Caution is advised on for any surgical procedures during the study. The appropriate interval of time between surgery and Compound 1 administration required to minimize the risk of impaired wound healing and bleeding has not been determined. Stopping Compound 1 is recommended at least 7 days prior to surgery. Postoperatively, the decision to reinitiate Compound 1 treatment should be based on a clinical assessment of satisfactory wound healing and recovery from surgery.

### Dose Modification

Every effort should be made to administer study intervention on the planned dose and schedule. In the event of significant toxicity, dosing may be delayed and/or reduced as described below. In the event of multiple toxicities, dose modification should be based on the worst toxicity observed. Participants are to be instructed to notify investigators at the first occurrence of any adverse symptom.

Dose modifications may occur in one of three ways:
Within a cycle: dosing interruption until adequate recovery and dose reduction, if required, during a given treatment cycle;
Between cycles: next cycle administration may be delayed due to persisting toxicity when a new cycle is due to start;
In the next cycle: dose reduction may be required in a subsequent cycle based on toxicity experienced in the previous cycle.

### Dosing Interruptions

With respect to study intervention, participants experiencing the AEs in Table 7 should follow the dosing guidelines provided accordingly.

Appropriate follow up assessments should be done until adequate recovery occurs as assessed by the investigator. Criteria required before treatment can resume are described in the Dose Reductions section.

Doses may be held up to 3 weeks until toxicity resolution. Depending on when the adverse event resolved, a treatment interruption may lead to the participant missing all subsequent planned doses within that same cycle or even to delay the initiation of the subsequent cycle. Dose interruption of week within a dosing cycle (2 weeks on/1 week off) will result in the start of the subsequent cycle upon resumption of dosing.

If the adverse event that led to the treatment interruption recovers in <1 week, then re dosing in that cycle is allowed. Doses omitted for toxicity are not replaced within the same cycle. The need for a dose reduction at the time of treatment resumption should be based on the criteria defined in the Dose Reductions section, unless expressly agreed otherwise following discussion between the investigator and the sponsor. If a dose reduction is applied in the same cycle, the participant will need to return to the clinic to receive new drug supply.

In the event of a treatment interruption for reasons other than treatment related toxicity (e.g., elective surgery) lasting >3 weeks, treatment resumption will be decided in consultation with the sponsor.

### Dose Reductions

Following dosing interruption or cycle delay due to toxicity, the Compound 1 dose may need to be reduced when treatment is resumed.

Investigators should always manage their participants according to their medical judgment based on the particular clinical circumstances.

Participants experiencing recurrent and intolerable Grade 2 toxicity may resume dosing at the next lower dose level once recovery to Grade or baseline is achieved.

Dose reduction of Compound 1 by 1 and, if needed and permissible, 2 dose levels (Table 6) will be allowed depending on the type and severity of toxicity encountered. Does reduction below 10 mg (Dose level −1) is not permitted. Participants requiring more than 2 dose reductions or dose reduction below 10 mg (Dose level −1) will be discontinued from the treatment and entered into the follow-up phase, unless otherwise agreed between the investigator and the sponsor. All dose modifications/adjustments must be clearly documented in the participant's source notes and CRF.

Once a dose has been reduced for a given participant, all subsequent cycles should be administered at that dose level, unless further dose reduction is required. Intraparticipant dose re-escalation is not allowed.

TABLE 6
Available Dose Levels

| | PF-07265807 | Dosing Schedule |
|---|---|---|
| Dose Level | Dose (mg, QD)* | (Cycle length = 21 days) |
| 5 | 300 | 2 weeks on/1 week off |
| 4 | 200 | |
| 3 | 100 | |
| 2 | 50 | |
| 1 | 25 | |
| (starting) | | |
| −1 | 10 | |

*Dose de-escalation of PF-07265807 may also occur within a range of 5 to 50 mg after discussion with the sponsor. PF-07265807 dose de-escalation below 10 mg QD is not allowed.

Participants experiencing a DLT may resume dosing at the next lower dose level (if applicable) once adequate recovery is achieved, and in the opinion of the investigator and sponsor, the participant is benefiting from therapy.

Recommended dose modifications for study intervention are described in Table 7.

TABLE 7
Dose Modifications for Study Intervention-Related Toxicity

| | | | |
|---|---|---|---|
| | Severity or Grade | | Study Treatment |
| Event | (CTCAE v5.0) | Management | and Follow-up |

Skin and Subcutaneous Tissue Disorders

| | | | |
|---|---|---|---|
| Rash | Grade 1 (i.e., | Initiate | Continue study |
| | covering <10% BSA) | symptomatic | intervention per |
| | | therapy per | protocol |
| | | institutional | |
| | | standard of care | |
| | Grade 2 (i.e., | Initiate | Hold study |
| | covering 10 to 30% | symptomatic | intervention until |
| | BSA) | therapy per | resolves to ≤ Grade 1 |
| | | institutional | then resume study |
| | | standard of care | intervention at the |
| | | | current dose |
| | Grade 3-4 (i.e., | Initiate | Hold study |
| | covering >30% BSA; | symptomatic | intervention |
| | life threatening) | therapy per | Grade 3 events |
| | | institutional | that improve |
| | | standard of care | to ≤ Grade 1 in <14 |
| | | Consider | consecutive days: |
| | | dermatology | May resume |
| | | consultation | study intervention at |
| | | | the next lower dose |
| | | | level |
| | | | If recurs |
| | | | participant should be |
| | | | permanently |
| | | | discontinued from |
| | | | study intervention |
| | | | For Grade 3 |
| | | | toxicity >14 |
| | | | consecutive |
| | | | days despite |
| | | | maximal treatment or |
| | | | any Grade 4 toxicity |
| | | | permanently |
| | | | discontinue study |
| | | | intervention |

Gastrointestinal Disorders

| | | | |
|---|---|---|---|
| Diarrhea | Grade 1 (i.e., <4 | Initiate | Continue study |
| | stools per day over | antidiarrheal and | intervention per |
| | baseline) | symptomatic | protocol |
| | | treatment per | |
| | | institutional | |
| | | standard of care | |
| | | Close | |
| | | monitoring for | |
| | | worsening | |
| | | symptoms | |
| | Grade 2 (i.e., 4-6 | Initiate | If persists for |
| | stools per day over | antidiarrheal and | more than 3 days on |
| | baseline) | symptomatic | optimal antidiarrheal |
| | | treatment per | treatment hold study |
| | | institutional | intervention until |
| | | standard of care | resolves to ≤ Grade 1 |
| | | | If improves to |
| | | | Grade 1 resume |
| | | | study intervention at |
| | | | the current dose |
| | | | If no improvement |
| | | | or worsening after |
| | | | holding study |
| | | | intervention for 3-5 |
| | | | days, consider other |
| | | | causes of diarrhea |
| | | | (infection, immune |
| | | | related, etc.) and |
| | | | treat per institutional |
| | | | standard of care |
| | Grade 3-4 (i.e., ≥7 | Initiate | Hold study |
| | stools per day over | antidiarrheal and | intervention |
| | baseline; life | symptomatic | If no improvement |
| | threatening) | treatment per | or worsening after |
| | | institutional | holding study |
| | | standard of care | intervention for 3-5 |
| | | Consider GI | days, consider other |
| | | consultation | causes of diarrhea |
| | | | (infection, immune |
| | | | related, etc.) and |
| | | | treat per institutional |
| | | | standard of care |
| | | | If Grade 3 diarrhea |
| | | | improves to ≤ Grade |
| | | | 1 within 48 |
| | | | hours, then resume |
| | | | study intervention at |
| | | | the next lower dose |
| | | | level |
| | | | If Grade 3 |
| | | | diarrhea does not |
| | | | improve to ≤ Grade 1 |
| | | | within 48 hours after |
| | | | maximal antidiarrheal |
| | | | treatment |
| | | | permanently |
| | | | discontinue study |
| | | | intervention |
| | | | Permanently |
| | | | discontinue study |
| | | | intervention for any |
| | | | Grade 4 toxicity |
| Nausea and | Grade 1-2 | Initiate anti- | Continue study |
| Vomiting | | nausea and | intervention per |
| | | vomiting treatment | protocol |
| | | per institutional | For Grade 2 |
| | | standard of care | events that persist for |
| | | | more than 3 days |
| | | | despite optimal anti- |
| | | | nausea/vomiting |
| | | | therapy, hold study |
| | | | intervention until |
| | | | resolves to ≤ Grade |
| | | | 1, then resume study |
| | | | intervention at the |
| | | | current dose or next |
| | | | lower dose level |
| | Grade 3-4 | Initiate anti- | For Grade 3 |
| | | nausea and | events that persist |
| | | vomiting treatment | for >48 hours despite |
| | | per institutional | optimal anti- |
| | | standard of care | nausea/vomiting |
| | | | therapy, hold study |
| | | | intervention until |
| | | | resolves to ≤ Grade |
| | | | 1, then resume study |
| | | | intervention at the |
| | | | next lower dose level |
| | | | Permanently |
| | | | discontinue study |
| | | | intervention for any |
| | | | Grade 4 toxicity |

Respiratory Disorders

| | | | |
|---|---|---|---|
| Interstitial | Grade 1 (i.e., | Monitor for | Hold study |
| Lung/Pneumonitis | radiographic changes | symptoms per | intervention |
| | only) | institutional | When resolved, |
| | | standard of care | resume study |
| | | Re-image per | intervention at the |
| | | institutional | current dose or next |
| | | standard of care | lower dose level |
| | | Consider | |
| | | pulmonary | |
| | | consultation | |
| | Grade 2 (i.e., mild to | Consider | Hold study |
| | moderate symptoms) | pulmonary | intervention |
| | | consultation | When resolved, |
| | | Monitor | resume study |
| | | symptoms daily, | intervention at the |
| | | consider | next lower dose level |
| | | hospitalization per | |
| | | institutional | |
| | | standard of care | |
| | | Start systemic | |
| | | steroids per | |
| | | institutional | |
| | | standard of care | |
| | | Under | |
| | | conditions | |
| | | requiring hold of | |
| | | study intervention, | |
| | | steroid use is | |
| | | allowed until | |
| | | resolved to | |
| | | baseline or < Grade | |
| | | 1. Once | |
| | | the steroid dose | |
| | | has been tapered | |
| | | down to a low | |
| | | dose or off, study | |
| | | intervention may | |
| | | be resumed | |
| | | Re-image per | |
| | | institutional | |
| | | standard of care | |
| | Grade 3-4 (i.e., | Hospitalize the | Permanently |
| | severe symptoms, | participant | discontinue study |
| | new or worsening | Consider | intervention |
| | hypoxia; life- | pulmonary | |
| | threatening) | consultation | |
| | | Start systemic | |
| | | steroids and | |
| | | antibiotics per | |
| | | institutional | |
| | | standard of care | |
| | | Re-image per | |
| | | institutional | |
| | | standard of care | |
| | | If not | |
| | | improving after 48 | |
| | | hours consider | |
| | | adding additional | |
| | | immunosuppressive | |
| | | agents | |

Hepatic Adverse Events

| | | | |
|---|---|---|---|
| AST/ALT | Grade 1 (AST or ALT > | Continue | Continue study |
| Elevations | ULN - 3.0 × ULN if | monitoring per | intervention per |
| | baseline was normal; | protocol | protocol |
| | 1.5-3.0 × baseline if | | |
| | baseline was | | |
| | abnormal) | | |
| | Grade 2 (AST or ALT > | Increase | Continue study |
| | 3.0-5.0 × ULN if | monitoring and | intervention per |
| | baseline was normal; > | initiate treatment | protocol |
| | 3.0-5.0 × baseline | per institutional | If resolves to ≤ Grade |
| | if baseline was | standard of care | 1 or baseline |
| | abnormal) | | in ≤14 days, |
| | | | maintain dose level |
| | | | of study intervention |
| | | | If not resolved to ≤ Grade |
| | | | 1 or baseline |
| | | | in ≤14 days, hold |
| | | | study intervention |
| | | | until resolved |
| | | | to ≤ Grade 1 or |
| | | | baseline, then |
| | | | resume study |
| | | | intervention at |
| | | | current dose level |
| | | | If additional |
| | | | occurrence: |
| | | | Hold study |
| | | | intervention until |
| | | | resolved to ≤ Grade 1 |
| | | | or baseline, then |
| | | | resume study |
| | | | intervention at the |
| | | | next lower dose level |
| | Grade 3: | Increase | Continue study |
| | AST or ALT | monitoring and | intervention per |
| | (>5.0-8.0 × ULN | initiate treatment | protocol |
| | if baseline was | per institutional | If resolved to ≤ Grade |
| | normal; >5.0-8.0 × | standard of care | 1 or baseline |
| | baseline if | | in 7-10 days, |
| | baseline was | | maintain dose level |
| | abnormal) | | of study intervention |
| | | | If not resolved to ≤ Grade |
| | | | 1 or baseline |
| | | | in 7-10 days, study |
| | | | intervention should |
| | | | be discontinued or |
| | | | consult with study |
| | | | medical monitor |
| | | | If additional |
| | | | occurrence: |
| | | | Hold study |
| | | | intervention until |
| | | | resolved to ≤ Grade 1 |
| | | | or baseline, then |
| | | | resume treatment at |
| | | | the next lower dose |
| | | | level |
| | Grade 3: | Increase | Hold study |
| | AST or ALT >8.0-20× | monitoring and | intervention until |
| | ULN if baseline was | initiate treatment | resolved to ≤ Grade 1 |
| | normal; >8.0-20.0× | per institutional | or baseline |
| | baseline if | standard of care | If resolved to ≤ Grade |
| | baseline was | | 1 or baseline |
| | abnormal | | in 7-10 days, resume |
| | | | study intervention at |
| | | | the next lower dose |
| | | | level |
| | | | If not resolved to ≤ Grade |
| | | | 1 or baseline |
| | | | in 7-10 days, |
| | | | permanently |
| | | | discontinue study |
| | | | intervention |
| | Grade 3: | Increase | Permanently |
| | Any Grade 3 | monitoring and | discontinue study |
| | AST/ALT and | initiate treatment | intervention |
| | Total Bilirubin ≥ | per institutional | |
| | Grade 2 | standard of care | |
| | Grade 4 (AST or ALT > | Increase | Permanently |
| | 20.0 × | monitoring and | discontinue study |
| | ULN if baseline | initiate treatment | intervention |
| | was normal; >20 × | per institutional | |
| | baseline if | standard of care | |
| | baseline was | | |
| | abnormal) | | |
| Total Bilirubin | Grade 1 (>ULN - | Increase | Continue study |
| Elevations | 1.5 × ULN if baseline | monitoring and | intervention per |
| without | was normal; >1.0-1.5 × | initiate treatment | protocol |
| ALT/AST | baseline if | per institutional | If resolved to < Grade |
| elevation | baseline was | standard of care | 1 or baseline |
| (if ALT/AST is | abnormal) | | in ≤14 days, |
| also elevated, | | | maintain dose level |
| see AST/ALT | | | of study intervention |
| Elevations) | | | If not resolved to < Grade |
| | | | 1 or baseline |
| | | | in ≤14 days, hold |
| | | | study intervention |
| | | | until resolved, then |
| | | | resume study |
| | | | intervention at |
| | | | current dose level |
| | | | If additional |
| | | | occurrence: |
| | | | Hold study |
| | | | intervention until |
| | | | resolved to < Grade 1 |
| | | | or baseline, then |
| | | | resume study |
| | | | intervention at the |
| | | | next lower dose level |
| | Grade 2 (>1.5-3.0 × | Increase | Hold study |
| | ULN if baseline was | monitoring and | intervention until |
| | normal; >1.5-3.0 × | initiate treatment | resolved to Grade ≤1 |
| | baseline if baseline | per institutional | or baseline |
| | was abnormal) | standard of care | If resolved to |
| | | | Grade ≤1 or baseline |
| | | | in 7-10 days, resume |
| | | | study intervention at |
| | | | the next lower dose |
| | | | level |
| | | | If not resolved to |
| | | | Grade ≤1 or baseline |
| | | | in 7-10 days, |
| | | | permanently |
| | | | discontinue study |
| | | | intervention |
| | Grade 3 (>3.0 × ULN | Increase | Permanently |
| | if baseline was | monitoring and | discontinue study |
| | normal; >3.0 × | initiate treatment | intervention |
| | baseline if baseline | per institutional | |
| | was abnormal) | standard of care | |
| | Grade 4 (>10.0 × | Increase | Permanently |
| | ULN if baseline was | monitoring and | discontinue study |
| | normal; >10.0 × | initiate treatment | intervention |
| | baseline if baseline | per institutional | |
| | was abnormal) | standard of care | |

Renal Disorders

| | | | |
|---|---|---|---|
| Serum | Grade 1 (>1-1.5 × | Continue | Continue study |
| Creatinine | baseline; >ULN-1.5 × | monitoring per | intervention per |
| Increased | ULN) | protocol | protocol |
| | Grade 2 (>1.5-3.0 × | Increase | Continue study |
| | baseline; >1.5-3.0 × | monitoring of | intervention per |
| | ULN) | serum creatinine | protocol or hold study |
| | | per institutional | intervention until |
| | | standard of care | resolved to ≤ Grade 1 |
| | | Decision to | or baseline then |
| | | continue or hold | resume study |
| | | study intervention | intervention |
| | | will be at the | |
| | | discretion of the | |
| | | investigator based | |
| | | on underlying | |
| | | etiology and best | |
| | | medical judgement | |
| | | in consultation | |
| | | with the study | |
| | | medical monitor | |
| | Grade 3 (>3.0 × | Increase | Hold study |
| | baseline; >3.0-6.0 × | monitoring of | intervention until |
| | ULN) | serum creatinine | resolved to < Grade 1 |
| | | per institutional | or baseline then |
| | | standard of care | resume study |
| | | Consider | intervention at the |
| | | nephrology | next lower dose level |
| | | consultation | If not resolved or |
| | | | worsens, consider |
| | | | nephrology |
| | | | consultation |
| | Grade 4 (>6.0 × | Increase | Permanently |
| | ULN) | monitoring of | discontinue study |
| | | serum creatinine | intervention |
| | | per institutional | |
| | | standard of care | |
| | | Consider | |
| | | nephrology | |
| | | consultation | |

Cardiac Disorders

| | | | |
|---|---|---|---|
| QTcF | Grade 3 | Verify that | Permanently |
| Prolongation | (i.e., QTc ≥501 ms; >60 | QTcF prolonged | discontinue study |
| | ms change from | has been seen on | intervention |
| | baseline on at least | 2 separate ECGs | |
| | two separate ECGs) | taken at least 5 | |
| | | minutes apart | |
| | | Increase | |
| | | cardiac monitoring | |
| | | and initiate | |
| | | treatment per | |
| | | institutional | |
| | | standard of care | |
| | | Consider | |
| | | cardiologist | |
| | | consultation | |
| | Grade 4 (torsade de | Hospitalize | Permanently |
| | pointes; polymorphic | participant | discontinue study |
| | ventricular | Increase | intervention |
| | tachycardia; | cardiac monitoring | |
| | signs/symptoms of | and initiate | |
| | serious arrhythmia) | treatment per | |
| | | institutional | |
| | | standard of care | |
| | | Consider | |
| | | cardiologist | |
| | | consultation | |

Blood and Lymphatic System Disorders

| | | |
|---|---|---|
| Grade 3 | Increase | Hold study |
| | monitoring and | intervention until |
| | initiate treatment | resolved to ≤ Grade |
| | per institutional | 1, then resume study |
| | standard of care | intervention at the |
| | | next lower dose level |
| Grade 4 | Increase | Permanently |
| | monitoring and | discontinue study |
| | initiate treatment | intervention if the |
| | per institutional | laboratory changes |
| | standard of care | are associated with |
| | | an AE |

Ocular Toxicities

| | | | |
|---|---|---|---|
| Retinal | Grade 1 | Repeat | Continue study |
| Events | | ophthalmic | intervention per |
| | | monitoring | protocol |
| | | assessments as | If participant is |
| | | outlined per | asymptomatic, |
| | | protocol within | maintain dose level |
| | | 7-10 days | and continue visual |
| | | Counsel | assessments as |
| | | participant to | outlined per protocol |
| | | report any new | If participant |
| | | visual symptoms | becomes |
| | | immediately | symptomatic (blurred |
| | | | vision, photophobia, |
| | | | etc.) or visual acuity |
| | | | assessment shows |
| | | | worsening vision, see |
| | | | Grade 2 guidance |
| | Grade 2 | Repeat | Hold study |
| | | ophthalmic | intervention |
| | | monitoring | If not resolved to |
| | | assessments as | baseline or Grade ≤1, |
| | | outlined per | continue to hold |
| | | protocol within | study intervention |
| | | 7-10 days | and repeat |
| | | Counsel | ophthalmic |
| | | patient to report | assessments in 7-10 |
| | | any new visual | days |
| | | symptoms | If resolved to |
| | | immediately | baseline or Grade ≤1 |
| | | | and associated with |
| | | | reversible retinal |
| | | | effects, resume study |
| | | | intervention as the |
| | | | next lower dose level |
| | | | and continue with |
| | | | visual assessments |
| | | | as outlined per |
| | | | protocol |
| | | | If remains Grade |
| | | | 2 or higher or |
| | | | associated with |
| | | | irreversible retinal |
| | | | effects, permanently |
| | | | discontinue study |
| | | | intervention |
| | Grade 3 | Repeat | Hold study |
| | | ophthalmic | intervention |
| | | monitoring | If not resolved to |
| | | assessments as | baseline or Grade ≤1, |
| | | outlined per | continue to hold |
| | | protocol within | study intervention |
| | | 7-10 days | and repeat |
| | | Counsel | ophthalmic |
| | | participant to | assessments in 7-10 |
| | | report any new | days. |
| | | visual symptoms | If resolved to |
| | | immediately | baseline or Grade ≤1 |
| | | | and associated with |
| | | | reversible retinal |
| | | | effects, resume study |
| | | | intervention at the |
| | | | next lower dose level |
| | | | and continue with |
| | | | visual assessments |
| | | | as outlined per |
| | | | protocol |
| | | | If remains Grade |
| | | | 3 or higher or |
| | | | associated with |
| | | | irreversible retinal |
| | | | effects, permanently |
| | | | discontinue study |
| | | | intervention |
| | Grade 4 | Repeat | Permanently |
| | | ophthalmic | discontinue study |
| | | monitoring | intervention |
| | | assessments as | Follow-up with |
| | | outlined per | ophthalmic |
| | | protocol within | monitoring until |
| | | 7-10 days | stabilization or |
| | | | resolution |

All Other Adverse Events

| | | |
|---|---|---|
| Grade 1 (mild) | Initiate | Continue study |
| | treatment and | intervention per |
| | monitor per | protocol |
| | institutional | |
| | standard of care | |
| Grade 2 (moderate) | Initiate | First occurrence: |
| | treatment and | Continue study |
| | monitor per | intervention per |
| | institutional | protocol |
| | standard of care | Second |
| | Consider | occurrence: Hold |
| | appropriate | study intervention |
| | specialty | until resolved |
| | consultation as | to ≤ Grade 1, then |
| | applicable | resume study |
| | Resume study | intervention at the |
| | intervention at the | current dose or the |
| | current dose or the | next lower dose level |
| | next lower dose | |
| | level is at the | |
| | discretion of the | |
| | investigator based | |
| | on underlying | |
| | etiology and best | |
| | medical judgement | |
| | in consultation | |
| | with the study | |
| | medical monitor. | |
| Grade 3 (severe) | Initiate | First occurrence: |
| | treatment and | Hold study |
| | monitor per | intervention until |
| | institutional | resolved to ≤ Grade |
| | standard of care | 1, then resume study |
| | Consider | intervention at the |
| | appropriate | next lower dose level |
| | specialty | Permanently |
| | consultation as | discontinue study |
| | applicable | intervention if the |
| | | toxicity does not |
| | | resolve to ≤ Grade 1 |
| | | or baseline level |
| | | within 28 days of last |
| | | dose, except by |
| | | approval of the |
| | | Investigator and the |
| | | Array Medical |
| | | Monitor |
| | | Permanently |
| | | discontinue study |
| | | intervention for a |
| | | second occurrence of |
| | | previously resolved |
| | | Grade 3 AE |
| Grade 4 (life | Initiate | Permanently |
| threatening) | treatment and | discontinue study |
| | monitor per | intervention |
| | institutional | |
| | standard of care | |
| | Consider | |
| | appropriate | |
| | specialty | |
| | consultation as | |
| | applicable | |

### Tumor Response Assessments

Tumor assessments will include all known or suspected disease sites. Imaging will include contrast-enhanced chest, abdomen and pelvis computed tomography or MRI scans; brain computed tomography or MRI scan for participants with known or suspected brain metastases; bone scan and/or bone x-rays for participants with known or suspected bone metastases. For participants with known computed tomography contrast allergy, a non-contrast computed tomography of the chest with contrast enhanced abdominal and pelvic MRI can be used. The same imaging technique used to characterize each identified and reported lesion at baseline will be employed in the following tumor assessments.

Anti-tumor activity will be assessed through radiological tumor assessments conducted at baseline, during treatment, whenever disease progression is suspected (e.g., symptomatic deterioration), and at the time of withdrawal from treatment (if not done in the previous 6 weeks).

Assessment of response will be made using RECIST version 1.19 (Table 8 and Table 9).

All participants' files and radiologic images must be available for source verification and for potential peer review.

TABLE 8
Objective Response Status at Each Assessment for Participants
with Measurable Disease at Baseline (RECIST Version 1.1)

| Target | | | Objective |
|---|---|---|---|
| Lesions | Non-target Lesions | New Lesions | status |
| CR | CR | No | CR |
| CR | Non-CR/Non-PD or | No | PR |
| | not all evaluated | | |
| PR | Non-PD* or not | No | PR |
| | all evaluated | | |
| SD | Non-PD* or not | No | SD |
| | all evaluated | | |
| Not all | Non-PD | No | NE |
| evaluated | | | |
| PD | Any | Yes or No | PD |
| Any | PD | Yes or No | PD |
| Any | Any | Yes** | PD |

*Non-PD includes CR and Non-CR/Non-PD
**New lesions must be unequivocal

TABLE 9
Objective Response Status at Each Assessment for Participants
with Non-Target Disease Only (RECIST Version 1.1)

| Non-target Disease | New Lesions | Objective status |
|---|---|---|
| CR | No | CR |
| Non-CR/Non-PD | No | Non-CR/Non-PD |
| Not all evaluated | No | NE |
| Unequivocal progression | Yes or No | PD |
| Any | Yes* | PD |

*New lesions must be unequivocal

### Ophthalmic Assessments

An ophthalmic examination (including best-corrected visual acuity, biomicroscopy, intraocular pressure, fundoscopy, fundus photography, fundus autofluorescence photography and OCT) will be performed by an ophthalmologist, and preferably, the same ophthalmologist for each individual participant at each timepoint. Ophthalmic assessments must meet parameters at screening as specified in the eligibility criteria for enrollment on study. The ophthalmic evaluation should be repeated at any point during the treatment period if clinically indicated. Participants will be instructed to report new visual changes immediately and not wait until the next scheduled clinic visit. All images and results will be made available to the sponsor.

In addition to the ophthalmologist, an independent central reader will be utilized for OCT images. Abnormal findings reported from designated central reader on OCT are to be reported as an AE if agreed on based upon review by the ophthalmologist, investigator, and sponsor.

The timing and extent of the ophthalmic assessments may be modified based on emerging safety data.

| Abbreviation | Term |
|---|---|
| AE | adverse event |
| AIDS | acquired immunodeficiency syndrome |
| ALT | alanine aminotransferase |
| ANC | absolute neutrophil count |
| ASCO | American Society of Clinical Oncology |
| AST | aspartate aminotransferase |
| AUCₗₐₛₜ | area under the curve from the time of dose to the |
| | last measurable concentration |
| AUCₜₐᵤ | area under the curve from the time of dose to the |
| | time of the subsequent dose |
| AUC_{inf} | area under the curve from the time of dose |
| | extrapolated to infinity |
| AV | atrioventricular |
| BCRP | breast cancer resistance protein |
| BSA | body surface area |
| CHF | congestive heart failure |
| CL/F | apparent oral clearance |
| Cₘₐₓ | maximum observed concentration |
| C_{ss, max} | maximum steady-state concentration |
| CONSORT | Consolidated Standards of Reporting Trials |
| CR | complete response |
| CRF | case report form |
| CTCAE | Common Terminology Criteria for Adverse Events |
| DCR | disease control rate |
| DLT | dose-limiting toxicity |
| DOR | duration of response |
| ECG | electrocardiogram |
| ECOG | Eastern Cooperative Oncology Group |
| FFPE | formalin-fixed paraffin-embedded |
| GI | gastrointestinal |
| HBV | hepatitis B virus |
| HCC | hepatocellular carcinoma |
| HCV | hepatitis C virus |
| HIV | human immunodeficiency virus |
| HNSCC | head and neck squamous cell carcinoma |
| IMP | investigational medicinal product |
| IOP | intraocular pressure |
| LBBB | left bundle branch block |
| MRI | magnetic resonance imaging |
| MSI-H | microsatellite instability-high |
| MTD | maximum tolerated dose |
| NCI | National Cancer Institute |
| NE | not evaluated |
| NIMP | non-investigational medicinal product |
| NSCLC | non-small cell lung cancer |
| OCT | optical coherence tomography |
| PD | progressive disease |
| QD | once daily |
| QTc | corrected QT |
| QTcF | corrected QT (Fridericia method) |
| R_{ac} | accumulation ratio |
| RCC | renal cell carcinoma |
| RECIST | Response Evaluation Criteria in Solid Tumors |
| RP2D | recommended phase 2 dose |
| SAE | serious adverse event |
| SCLC | small cell lung cancer |
| SD | stable disease |
| STD10 | severely toxic dose in 10% of the animals |
| t_{1/2} | terminal elimination half-life |
| Tₘₐₓ | time to maximum concentration |
| T_{ss, max} | time to maximum concentration at steady state |
| | during dosing interval |
| ULN | upper limit of normal |
| V_{z/F} | apparent terminal volume of distribution |

What is claimed is:

1-41. (canceled)

42. A method for treating cancer comprising administering to a patient in need thereof, over a period of time, a therapeutically effective amount of a compound which is (R)—N-(3-fluoro-4-((3-((1-hydroxypropan-2-yl)amino)-1H-pyrazolo[3,4-b]pyridin-4-yl)oxy)phenyl)-3-(4-fluorophenyl)-1-isopropyl-2,4-dioxo-1,2,3,4-tetrahydropyrimidine-5-carboxamide (Compound 1) or a pharmaceutically acceptable salt thereof as a monotherapy, wherein Compound 1 or a pharmaceutically acceptable salt thereof is administered according to an intermittent dosing schedule of at least one dosing cycle, wherein each dosing cycle comprises (a) a dosing period during which said Compound 1 or a pharmaceutically acceptable salt thereof is administered, and (b) a resting period during which said Compound 1 or a pharmaceutically acceptable salt thereof is not administered, wherein said dosing cycle is a 21-day cycle comprising (a) a dosing period wherein said Compound 1 or a pharmaceutically acceptable salt thereof is administered on days 1 through 14 of said 21-day cycle, and (b) a resting period wherein said Compound 1 or a pharmaceutically acceptable salt thereof is not administered, wherein said resting period is days 15-21.

43. A method for treating cancer comprising administering to a patient in need thereof, over a period of time, a therapeutically effective amount of Compound 1 or a pharmaceutically acceptable salt thereof as a monotherapy, wherein Compound 1 or a pharmaceutically acceptable salt thereof is administered according to an intermittent dosing schedule of at least one dosing cycle, wherein each dosing cycle comprises (a) a dosing period during which said Compound 1 or a pharmaceutically acceptable salt thereof is administered, and (b) a resting period during which said Compound 1 or a pharmaceutically acceptable salt thereof is not administered, wherein said dosing cycle is a 28-day cycle comprising:
(i) (a) a dosing period during which said Compound 1 or a pharmaceutically acceptable salt thereof is administered on days 1-14, and (b) a resting period during which said Compound 1 or a pharmaceutically acceptable salt thereof is not administered on days 15-28; or
(ii) (a) a dosing period during which said Compound 1 or a pharmaceutically acceptable salt thereof is administered on days 1-7 and days 15-21, and (b) a resting period during which said Compound 1 or a pharmaceutically acceptable salt thereof is not administered on days 8-14 and 22-28.

44. A method for treating cancer comprising administering to a patient in need thereof, over a period of time, a therapeutically effective amount of Compound 1 or a pharmaceutically acceptable salt thereof and a PD-1 inhibitor according to an intermittent dosing schedule of at least one dosing cycle, wherein each dosing cycle comprises (a) a dosing period during which said Compound 1 or a pharmaceutically acceptable salt thereof and said PD-1 inhibitor are administered, and (b) a resting period during which said Compound 1 or a pharmaceutically acceptable salt thereof and said PD-1 inhibitor are not administered, wherein said dosing cycle is a 28-day cycle comprising:
i) (a) a dosing period wherein said Compound 1 or a pharmaceutically acceptable salt thereof is administered on days 1 through 21 and said PD-1 inhibitor is administered on day 1, and (b) a resting period wherein said Compound 1 or a pharmaceutically acceptable salt thereof and said PD-1 inhibitor are not administered on days 22 through 28;
ii) (a) a dosing period wherein said Compound 1 or a pharmaceutically acceptable salt thereof is administered on days 1 through 21 and said PD-1 inhibitor is administered on day 1, and (b) a resting period wherein said Compound 1 or a pharmaceutically acceptable salt thereof and said PD-1 inhibitor are not administered on days 22 through 28;
iii) (a) a dosing period wherein said Compound 1 or a pharmaceutically acceptable salt thereof is administered on days 1 through 7 and said PD-1 inhibitor is administered on day 1, or day 1 and day 15, and (b) a resting period wherein said Compound 1 or a pharmaceutically acceptable salt thereof and said PD-1 inhibitor are not administered on days 16 through 28;
iv) (a) a dosing period wherein said Compound 1 or a pharmaceutically acceptable salt thereof is administered on days 1 through 14 and said PD-1 inhibitor is administered on day 1, or day 1 and day 15, and (b) a resting period wherein said Compound 1 or a pharmaceutically acceptable salt thereof and said PD-1 inhibitor are not administered on days 16 through 28; or
v) (a) a dosing period wherein said Compound 1 or a pharmaceutically acceptable salt thereof is administered on days 1 through 7 and days 15 through 21 and said PD-1 inhibitor is administered on day 1, or day 1 and day 15, and (b) a resting period wherein said Compound 1 or a pharmaceutically acceptable salt thereof and said PD-1 inhibitor are not administered on days 8 through 14 and days 22 through 28.

45. The method according to claim 44, wherein said PD-1 inhibitor is nivolumab or a biosimilar thereof.

46. The method according to claim 45, wherein said nivolumab or a biosimilar thereof is administered intravenously at a dose of about 3 mg/kg or as a flat dose of about 240 mg over 30 minutes.

47. The method according to claim 44, wherein said PD-1 inhibitor is sasanlimab or a biosimilar thereof.

48. A method for treating cancer comprising administering to a patient in need thereof, over a period of time, a therapeutically effective amount of Compound 1 or a pharmaceutically acceptable salt thereof and a PD-1 inhibitor according to an intermittent dosing schedule of at least one dosing cycle, wherein said dosing cycle is a 21-day cycle comprising:
i) (a) a dosing period wherein said Compound 1 or a pharmaceutically acceptable salt thereof is administered on days 1 through 14 and said PD-1 inhibitor is administered on day 1, and (b) a resting period wherein said Compound 1 or a pharmaceutically acceptable salt thereof and said PD-1 inhibitor are not administered, wherein the resting period is days 15 through 21,
ii) (a) a dosing period wherein said Compound 1 or a pharmaceutically acceptable salt thereof is administered on days 1 through 7 and said PD-1 inhibitor is administered on day 1, and (b) a resting period wherein said Compound 1 or a pharmaceutically acceptable salt thereof and said PD-1 inhibitor are not administered, wherein the resting period is days 8 through 21, or
iii) (a) a dosing period wherein said Compound 1 or a pharmaceutically acceptable salt thereof is administered on days 1 through 7 and days 15 through 21 and said PD-1 inhibitor is administered on day 1, and (b) a resting period wherein said Compound 1 or a pharmaceutically acceptable salt thereof and said PD-1 inhibitor are not administered, wherein the resting period is days 8 through 14.

49. The method according to claim 48, wherein said PD-1 inhibitor is pembrolizumab or a biosimilar thereof.

50. The method according to claim 49, wherein said pembrolizumab or a biosimilar thereof is administered intravenously at a dose of about 2 mg/kg or as a flat dose of about 200 mg over 30 minutes.

51. A method for treating cancer comprising administering to a patient in need thereof, over a period of time, a therapeutically effective amount of said Compound 1 or a pharmaceutically acceptable salt thereof and a PD-L1 inhibitor according to an intermittent dosing schedule of at least one dosing cycle, wherein each dosing cycle comprises (a) a dosing period during which said Compound 1 or a pharmaceutically acceptable salt thereof and said PD-L1 inhibitor are administered, and (b) a resting period during which said Compound 1 or a pharmaceutically acceptable salt thereof and said PD-L1 inhibitor are not administered.

52. The method of claim 51, wherein said dosing cycle is a 21-day cycle.

53. The method of claim 51, wherein said dosing cycle is a 28-day cycle wherein each of said dosing cycles comprises:
i) (a) a dosing period wherein said Compound 1 or a pharmaceutically acceptable salt thereof is administered on days 1 through 21, said PD-L1 inhibitor is administered on day 1, or day 1 and day 15, and (b) a resting period wherein said Compound 1 or a pharmaceutically acceptable salt thereof and said PD-L1 inhibitor are not administered, wherein the resting period is days 22 through 28,
ii) (a) a dosing period wherein said Compound 1 or a pharmaceutically acceptable salt thereof is administered on days 1 through 21, said PD-L1 inhibitor is administered on day 1, and (b) a resting period wherein said Compound 1 or a pharmaceutically acceptable salt thereof and said PD-L1 inhibitor are not administered, wherein the resting period is days 22 through 28,
iii) (a) a dosing period wherein said Compound 1 or a pharmaceutically acceptable salt thereof is administered on days 1 through 7 and said PD-L1 inhibitor is administered on day 1 and day 15, and (b) a resting period wherein said Compound 1 or a pharmaceutically acceptable salt thereof and said PD-L1 inhibitor are not administered, wherein the resting period is days 16 through 28,
iv) (a) a dosing period wherein said Compound 1 or a pharmaceutically acceptable salt thereof is administered on days 1 through 14 and said PD-L1 inhibitor is administered on day 1 and day 15, and (b) a resting period wherein said Compound 1 or a pharmaceutically acceptable salt thereof and said PD-L1 inhibitor are not administered, wherein the resting period is days 16 through 28, or
v) (a) a dosing period wherein said Compound 1 or a pharmaceutically acceptable salt thereof is administered on days 1 through 7 and days 15 through 21, said PD-L1 inhibitor is administered on day 1 and day 15, and (b) a resting period wherein said Compound 1 or a pharmaceutically acceptable salt thereof and said PD-L1 inhibitor are not administered, wherein the resting period is days 8 through 14 and 22 through 28.

54. The method according to claim 53, wherein said PD-L1 inhibitor is atezolizumab or a biosimilar thereof.

55. The method according to claim 54, wherein said atezolizumab or a biosimilar thereof is administered intravenously at a dose of about 840 mg every two weeks.

56. The method according to claim 42, wherein said cancer is a TAM-associated cancer.

57. The method according to claim 42, wherein said cancer is selected from the group consisting of is selected from colorectal cancer (CRC), bladder cancer, cervical cancer, gastric cancer, esophageal cancer, hepatocellular carcinoma (HCC), melanoma (e.g., mucosal or cutaneous), Merkel Cell Carcinoma, microsatellite instability-high (MSI-H) tumors, non-small cell lung cancer (NSCLC), head and neck squamous cell carcinoma, small cell lung cancer, renal cell carcinoma (RCC), or urothelial carcinoma.

58. The method according to claim 57, where said cancer is colorectal cancer

59. The method according to claim 42, wherein said Compound 1 is administered as a free base.
